(19) United States
(12) Patent Application Publication
(10) Pub. No.: US 2019/0030009 A1
(43) Pub. Date: Jan. 31, 2019

(54) COMPOUNDS AND COMPOSITIONS FOR THE TREATMENT OF CRYPTOSPORIDIOSIS

(71) Applicant: Novartis AG, Basel (CH)

(72) Inventors: Thierry Tidiane DIAGANA, Berkeley, CA (US); Manjunatha UJJINI, San Ramon, CA (US)

(21) Appl. No.: 16/072,060

(22) PCT Filed: Jan. 20, 2017
(86) PCT No.: PCT/IB2017/050319

§ 371 Date: Jul. 23, 2018

(30) Foreign Application Priority Data
Jan. 21, 2016 (SG) 10201600499R

(51) Int. Cl. A61K 31/444; A61K 31/437; A61K 31/538; A61K 31/506; A61P 1/00

(52) U.S. Cl. CPC A61K 31/444; A61K 31/437; A61P 1/00; A61K 31/506; A61K 31/538

(57) ABSTRACT

The invention relates to a method for treating, preventing, inhibiting, ameliorating, or eradicating the pathology and/or symptomology of cryptosporidiosis by administering a therapeutic agent which antagonizes or modulates the activity of phosphatidylinositol-4-OH kinase (PI4K), a lipid kinase of the cryptosporidium protozoa. In one embodiment, the therapeutic agent is a pyrazolo[1,5-a]pyridine compound of Formula I:

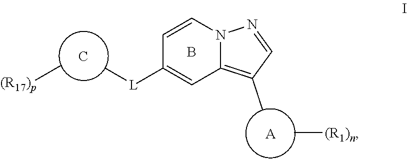

or a pharmaceutically acceptable salt, tautomer, or stereoisomer, thereof, wherein the variables are as defined herein.

### BACKGROUND OF THE INVENTION

### Field of the Invention

The invention relates to a method for treating, preventing, inhibiting, ameliorating, or eradicating the pathology and/or symptomology of cryptosporidiosis. The therapeutic agent is a small molecular inhibitor which antagonize or modulate the activity of phosphatidylinositol-4-OH kinase (PI4K), a lipid kinase of the cryptosporidium protozoa.

### Background

Globally ˜6.5 million children under the age of five die each year. Diarrhoeal diseases are the second leading cause of death in children and are responsible for ˜760,000 deaths in low income countries (2013). Nearly 80% of child deaths by diarrhea occur in South Asia and sub-Saharan Africa. Diarrhea is caused by a wide-range of pathogens including viral (rotavirus, norovirus etc), bacterial (Shiegella, ETEC, Vibrio, Campylobacter, etc) and protozoan parasites (Giardia, Entameoba, Cryptosporidium, etc). Rotavirus is the leading cause of diarrheal disease accounting for ˜450,000 deaths but safe and effective vaccines are already available. Childhood mortality caused by a diarrhea causing protozoan parasite Cryptosporidium spp is being recognized of late (Striepen, 2013).

Apicomplexan parasites cause a range of important human diseases like malaria, cryptosporidiosis and toxoplasmosis, caused respectively by phylogenetically related parasites Plasmodium spp, Cryptosporidium spp and Toxoplasma gondii. Cryptosporidiosis affects people worldwide; it is an intestinal illness that manifests as watery diarrhea. In humans, the disease is caused by mainly two species Cryptosporidium hominis and Cryptosporidium parvum. In healthy adults, cryptosporidiosis is usually a self-limiting infection with symptoms lasting 1-2 weeks. On the contrary immunocompromised individuals are highly vulnerable to cryptosporidiosis and suffer from chronic, long-lasting life-threatening diarrhea. A recent epidemiological study investigating the cause and effect of diarrhea in children below 5 years of age identified cryptosporidiosis as the second most common pathogen responsible for severe diarrhea and is also associated with death in 12-23 months old young children (Kotloff et al., 2012). Cryptosporidium is known to cause nearly 100,000 deaths in children each year. Cryptosporidium infection is also associated with long-term growth faltering and cognitive deficiency (Kotloff et al., 2012, Striepen, 2013, Checkley et al., 2015). Cryptosporidiosis is still an underappreciated global health concern with no available vaccine and with only one FDA approved drug, Nitazoxanide (Alinia) (2003). The standard of care is suboptimal and unproven in needy patient population, i.e., 6-18 months' old malnourished children and immunocompromised patients (Checkley et al., 2015). Hence there is an unmet medical need to find highly effective drugs against Cryptosporidiosis.

A major advance in understanding the molecular biology of Cryptosporidium came from the genome sequencing of C. parvum (Abrahamsen et al., 2004) and C. hominis (Xu et al., 2004). The genomes of these two closely related species are similar (96-97% identity) with 4000 genes spread on 8 chromosomes. The genome of Cryptosporidium spp are substantially smaller than other apicomplexan protozoan parasites like Plasmodium falciparum (Gardner et al., 2002) with fewer introns and shorter non-coding regions. Although Cryptosporidium exhibit genetic divergence from other apicomplexan parasites like Plasmodium, a number of druggable molecular targets and pathways are conserved between apicomplexan protozoa (Abrahamsen et al., 2004, Xu et al., 2004). WO2014/078802 A1 describes pyrazolo[1,5-a]pyridine compounds which are effective in inhibiting the proliferation of Plasmodium parasite; these efforts is being leveraged in the fight against Cryptosporidiosis.

### SUMMARY OF THE INVENTION

The invention relates to a method for preventing, treating, inhibiting, ameliorating, or eradicating the pathology and/or symptomology of cryptosporidiosis by administering to an patient in need thereof, an effective amount of a compound of Formular I:

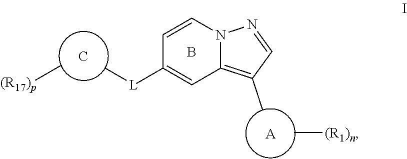

or a pharmaceutical acceptable salt, tautomer or stereoisomer thereof, wherein

n is 0, 1, 2 or 3;

p is 0, 1, 2, or 3;

L is selected from the group consisting of *—(CHR₃)₁₋₃—, *—CHR₃N(R₂)—, CHR₃O—, *—CHR₃S—, *—CHR₃S(O)—, *—CHR₃N(R₂)CHR₃—, *—C(O)—, *—C(O)N(R₂)—, C(O)N(R₂)CHR₃—, *—N(R₂)—, *—N(R₂)CHR₃—, *—N(R₂)C(O)—, *—N(R₂)C(O)N(R₂)—, N(R₂)S(O)₂—, and *—S(O)₂N(R₂)—, wherein
* represents the point of attachment of L to the pyrazolo[1,5-a]pyridine fused ring depicted in Formula I (Ring B);
each R₂ is selected from the group consisting of hydrogen, C₁₋₆alkyl, haloC₁₋₆alkyl, R—C₀₋₄alkylene, and R—C₀₋₄alkylene-C(O)—, wherein R is selected from the group consisting of hydroxyl, C₁₋₄alkoxy, amino, C₁₋₄alkylamino, C₃₋₆cycloalkyl, C₄₋₆heterocycloalkyl, and C₅₋₆heteroaryl, wherein the C₃₋₆cycloalkyl, C₄₋₆heterocycloalkyl, or C₅₋₆heteroaryl of R is unsubstituted or substituted by 1-2 substituents independently selected from the group consisting of halo, amino, hydroxyl, C₁₋₄alkyl, C₁₋₄alkoxy, oxo, and C₅₋₆heteroaryl; and
R₃ is hydrogen or C₁₋₄alkyl;

Ring A is C₆₋₁₀aryl or C₅₋₁₀heteroaryl;

Ring C is selected from the group consisting of C₆₋₁₀aryl, C₅₋₁₀heteroaryl, C₅₋₇cycloalkyl, C₅₋₇heterocycloalkyl, and a fused bicyclyl comprising a C₅₋₆heterocycloalky fused to a phenyl;

each R₁ is independently selected from the group consisting of halo, cyano, amino, C₁₋₄alkyl, C₁₋₄alkoxy, halo-C₁₋₄alkyl, —C(O)NR₇R₈, —NHC(O)R₁, phenyl, C₅₋₆heteroaryl, —C(O)R₁₁, —NHS(O)₂R₁₁, —S(O)₂R₁₁, and —S(O)₂NHR₈, wherein
the phenyl or C₅₋₆heteroaryl of R, is unsubstituted or substituted by 1-2 substituents independently selected from the group consisting of C₁₋₄alkyl, amino, halo, and C₁₋₄alkylamino;
R₇ is selected from the group consisting of hydrogen, C₁₋₄alkyl, and haloC₁₋₄alkyl;
R₈ is selected from the group consisting of hydrogen; haloC₁₋₄alkyl; C₃₋₆cycloalkyl; C₄₋₆heterocycloalkyl; C₁₋₄alkyl unsubstituted or substituted by hydroxy, amino, or C₁₋₄alkylamino; and
R₁₁ is selected from the group consisting of hydroxyl and C₁₋₆alkyl unsubstituted or substituted by 1-2 substituents independently selected from the group consisting of amino, C₃₋₆cycloalkyl, and C₄₋₆heterocycloalkyl;

each R₁₇ is selected from the group consisting of cyano, halo, C₁₋₄alkyl, halo-C₁₋₄alkyl, oxo, C₃₋₆cycloalkyl, —S(O)₂C₁₋₄alkyl; C₁₋₄alkoxy unsubstituted or substituted by hydroxy or amino; and —C(O)R₁₂, wherein R₁₂ is hydrogen, hydroxy or amino.

In a second aspect, the present invention relates to method for preventing, treating, inhibiting, ameliorating, or eradicating the pathology and/or symptomology of cryptosporidiosis by modulating the activity of phosphatidylinositol-4-OH kinase of the Cryptosporidium parasite.

Unless specified otherwise, the term “compound” refers to pyrazolo[1,5-a]pyridine compound of Fomula (I) or subformulae thereof, salts of the compound, hydrates or solvates of the compound, as well as all stereoisomers (including diastereoisomers and enantiomers), tautomers and isotopically labeled compound (including deuterium substitutions). A compound of Formula I (or subformulae thereof) further comprise polymorphs of the compound.

### DETAILED DESCRIPTION OF THE INVENTION

### Definitions

For purposes of interpreting this specification, the following definitions will apply and whenever appropriate, terms used in the singular will also include the plural and vice versa.

“Alkoxy” as used herein refers the radical —O-alkyl, wherein the alkyl is as defined herein. C_{X}alkoxy and C_{X-Y}alkoxy as used herein describe alkoxy groups where X and Y indicate the number of carbon atoms in the alkyl chain. Representative examples of C₁₋₁₀alkoxy include, but are not limited to, methoxy, ethoxy, propoxy, 2-propoxy, butoxy, tert-butoxy, pentyloxy, hexyloxy, heptyloxy, octyloxy and decyloxy. The alkyl portion of the alkoxy may be optionally substituted, and the substituents include those described for the alkyl group below.

“Alkyl” as used herein refers to a fully saturated branched or unbranched hydrocarbon chain having up to 10 carbon atoms. C_{X} alkyl and C_{X-Y} alkyl as used herein describe alkyl groups where X and Y indicate the number of carbon atoms in the alkyl chain. For example, C₁₋₁₀ alkyl refers to an alkyl radical as defined above containing one to ten carbon atoms. C₁₋₁₀ alkyl includes, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. Alkyl represented along with another radical like arylalkyl, heteroarylalkyl, alkoxyalkyl, alkoxyalkyl, alkylamino, where the alkyl portion shall have the same meaning as described for alkyl and is bonded to the other radical. For example, (C₆₋₁₀)aryl(C₁₋₃)alkyl includes, benzyl, phenylethyl, 1-phenylethyl, 3-phenylpropyl, 2-thienylmethyl, 2-pyridinylmethyl and the like.

Unless stated otherwise specifically in the specification, an alkyl group may be unsubstituted or substituted by one or more substituents to the extent that such substitution makes sense chemically. Typical substituents include, but are not limited to halo, hydroxyl, alkoxy, cyano, amino, acyl, aryl, arylalkyl, and cycloalkyl, or an heteroforms of one of these groups, and each of which can be substituted by the substituents that are appropriate for the particular group.

“Alkenyl” as used herein refers to a straight or branched, hydrocarbon chain having up to 10 carbon atoms and at least one carbon-carbon double bond. C_{X}alkenyl and C_{X-Y}alkenyl as used herein describe alkenyl groups where X and Y indicate the number of carbon atoms in the alkenyl chain. Examples of C₂₋₇alkenyl include vinyl, allyl, isopropenyl, pentenyl, hexenyl, heptenyl, 1-propenyl, 2-butenyl, 2-methyl-2-butenyl, and the like. The alkenyl may be optionally substituted, and the substituents include those described for the alkyl group descried herein.

“Alkylene” as used herein refers to a divalent alkyl group defined herein. Examples of C₁₋₁₀alkylene includes, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene. An alkylene group may be optionally substituted, and the substituents include those described for the alkyl group described herein.

“Amino” as used herein refers to the radical —NH₂. When an amino is described as “substituted” or “optionally substituted”, the term includes NR′R″ wherein each R′ and R″ is independently H, or is an alkyl, aryl, cycloalkyl, arylalkyl, cycloalkylalkyl group or a heteroform of one of these groups, and each of the alkyl, aryl, arylalkyl or cycloalkylalkyl groups or heteroforms of one of these groups, is optionally substituted with the substituents described herein as suitable for the corresponding group.

“Alkylamino” as used herein refers to the radical —NRₐR_{b}, where at least one of, or both, Rₐ and R_{b} are an alkyl group as described herein. An C1-4alkylamino group includes —NHC₁₋₄alkyl and —N(C₁₋₄alkyl)₂; e.g., —NHCH₃, —N(CH₃)₂, —NH(CH₂CH₃), —N(CH₂CH₃)₂, and the like.

“Aryl” as used herein refers to a 6-14 membered monocyclic or polycyclic aromatic ring assembly where all the ring atoms are carbon atoms. Typically, the aryl is a 6 membered monocyclic, a 10-12 membered bicyclic or a 14-membered fused tricyclic aromatic ring system. C_{X}aryl and C_{X-Y}aryl as used herein describe an aryl group where X and Y indicate the number of carbon atoms in the ring system. C₆₋₁₄aryls include, but are not limited to, phenyl, biphenyl, naphthyl, azulenyl, and anthracenyl.

An aryl may be unsubstituted or substituted by 1-5 (such as one, or two, or three) substituents independently selected from the group consisting of hydroxy, thiol, cyano, nitro, C₁₋₄alkyl, C₁₋₄alkenyl, C₁₋₄alkynyl, C₁₋₄alkoxy, thioC₁₋₄alkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄alkyl)amino, sulfonyl, sulfamoyl, alkylsulfamoyl, C₁₋₄alkylaminosulfonyl, aryl, heteroaryl, cycloalkyl and heterocycloalkyl, wherein each of the afore-mentioned substitutents may be further substituted by one or more substituents independently selected from halogen, alkyl, hydroxyl or C₁₋₄alkoxy groups.

When an “aryl” is represented along with another radical like “arylalkyl”, “aryloxyalkyl”, “aryloxycarbonyl”, “aryloxy-carbonylalkyl”, the aryl portion shall have the same meaning as described in the above-mentioned definition of “aryl”.

“Aryloxy” as used herein, refers to the radical —O-aryl, wherein aryl is as defined herein.

“Bicyclic” or “bicyclyl” as used here in refers to a ring assembly of two rings where the two rings are fused together, linked by a single bond or linked by two bridging atoms. The rings may be a carbocyclyl, a heterocyclyl, or a mixture thereof.

“Cycloalkyl”, as used herein, means a radical comprising a non-aromatic, saturated or partially unsaturated, monocyclic, bicyclic, tricyclic, fused, bridged or spiro polycyclic hydrocarbon ring system of 3-20 carbon atoms. C_{X}cycloalkyl and C_{X-Y}cycloalkyl are typically used where X and Y indicate the number of carbon atoms in the ring assembly. For example, C₃₋₆cycloalkyl includes cyclopropyl, cyclobutyl, cyclopentyl, cyclohexyl, cyclohexenyl, 2,5-cyclohexadienyl.

Exemplary monocyclic cycloalkyl include, but are not limited to, cyclopropyl, cyclobutyl, cyclopentyl, cyclopentenyl, cyclohexyl and cyclohexenyl and the like.

Exemplary bicyclic cycloalkyls include bornyl, norbornanyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl. Exemplary tricyclic cycloalkyl groups include, for example, adamantyl.

A cycloalkyl may be unsubstituted or substituted by one, or two, or three, or more substituents independently selected from the group consisting of hydroxyl, thiol, cyano, nitro, oxo, alkylimino, C₁₋₄alkyl, C₁₋₄alkenyl, C₁₋₄alkynyl, C₁₋₄alkoxy, C₁₋₄thioalkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄alkyl)amino, sulfonyl, sulfamoyl, alkylsulfamoyl, C₁₋₄alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁₋₄alkoxy groups.

“Cyano”, as used herein, refers to the radical —CN.

“EC50”, refers to the molar concentration of an inhibitor or modulator that produces 50% efficacy.

“Fused ring”, as used herein, refers to a multi-ring assembly wherein the rings comprising the ring assembly are so linked that the ring atoms that are common to two rings are directly bound to each other. The fused ring assemblies may be saturated, partially saturated, aromatics, carbocyclics, heterocyclics, and the like. Non-exclusive examples of common fused rings include decalin, naphthalene, anthracene, phenanthrene, indole, benzofuran, purine, quinoline, and the like.

“Halo” or “halogen” as used herein refers to fluoro, chloro, bromo, and iodo.

“Haloalkyl”, or halo-substituted-alkyl” as used herein, refers to an alkyl as defined herein, which is substituted by one or more halo atoms defined herein. The haloalkyl can be mono-haloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. C_{X}haloalkyl and C_{X-Y}haloalkyl are typically used where X and Y indicate the number of carbon atoms in the alkyl chain. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A C₁₋₄perhaloalkyl group refers to a C₁₋₄alkyl group having all hydrogen atoms replaced with halo atoms.

“Heteroaryl”, as used herein, refers to a 5-14 membered ring assembly (e.g., a 5-7 membered monocycle, an 8-10 membered bicycle, or a 13-14 membered tricyclic ring system) having 1 to 8 heteroatoms selected from N, O and S as ring atoms and the remaining ring atoms are carbon atoms. The nitrogen atoms of such heteroaryl rings can be optionally quaternerized and the sulfur atoms of such heteroaryl rings can be optionally oxidized. C_{X}heteroaryl and C_{X-Y}heteroaryl as used herein describe heteroaryls where X and Y indicate the number of ring atoms in the heteroaryl ring. Typical C₅₋₇heteroaryl groups include thienyl, furanyl, imidazolyl, pyrazolyl, pyrrolyl, pyrrolinyl, thiazolyl, 1,3,4-thiadiazolyl, isothiazolyl, oxazolyl, oxadiazole isoxazolyl, triazolyl, tetrazolyl, pyridyl, pyridazinyl, pyrazinyl, pyrazinyl, pyrimidinyl, and the like. Bicyclic or tricyclic C₈₋₁₄heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, quinazolinyle, pteridinyl, indolizine, imidazo[1,2a]pyridine, quinoline, quinolinyl, isoquinoline, phthalazine, quinoxaline, naphthyridine, naphthyridinyl, quinolizine, indolyl, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, purinyl, carbazole, acridine, phenazine, phenothiazene, phenoxazine, 1,2-dihydropyrrolo[3,2,1-hi]indole, indolizine, pyrido[1,2-a]indole and 2(1H)-pyridinone.

A heteroaryl may be unsubstituted or substituted with one or more substituents independently selected from hydroxyl, thiol, cyano, nitro, C₁₋₄alkyl, C₁₋₄alkenyl, C₁₋₄alkynyl, C₁₋₄alkoxy, thioC₁₋₄alkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄alkyl)amino, sulfonyl, sulfamoyl, alkylsulfamoyl, C₁₋₄alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁₋₄alkoxy groups.

When a heteroaryl is represented along with another radical like “heteroaryloxy”, “heteroaryloxyalkyl”, “heteroaryloxycarbonyl”, the heteroaryl portion shall have the same meaning as described in the above-mentioned definition of “heteroaryl”.

“Heteroaryloxy”, as used herein, refers to an —O-heteroaryl group, wherein the heteroaryl is as defined in this Application.

“Heteroatom”, as used herein, refers to an atom that is not a carbon atom.

Particular examples of heteroatoms include, but are not limited to nitrogen, oxygen, and sulfur.

“Heterocycloalkyl”, as used herein, refers to a 4-20 membered, non-aromatic, saturated or partially unsaturated, monocyclic or polycyclic ring system, comprising 1-8 heteroatoms as ring atoms and that the remaining ring atoms are carbon atoms. The heteroatoms are selected from N, O, and S, preferably 0 and N. The nitrogen atoms of the heterocycloalkyl can be optionally quaternerized and the sulfur atoms of the heterocycloalkyl can be optionally oxidized. The heterocycloalkyl can include fused or bridged rings as well as spirocyclic rings. C_{X}heterocycloalkyl and C_{X-Y}heterocycloalkyl are typically used where X and Y indicate the number of ring atoms in the ring. Typically, the. heterocycloalkyl is 4-8-membered monocyclic ring containing 1 to 3 heteroatoms, a 7 to 12-membered bicyclic ring system containing 1-5 heteroatoms, or a 10-15-membered tricyclic ring system containing 1 to 7 heteroatoms. Examples of C₄₋₆heterocycloalkyl include azetidinyl, tetrahydrofuran (THF), dihydrofuran, 1, 4-dioxane, morpholine, 1,4-dithiane, piperazine, piperidine, 1,3-dioxolane, imidazolidine, imidazoline, pyrazolidinyl, pyrroline, pyrrolidine, tetrahydropyran, dihydropyran, oxathiolane, dithiolane, 1,3-dioxane, 1,3-dithiane, oxathiane, thiomorpholine, and the like

A heterocycloalkyl may be unsubstituted or substituted with 1-5 substituents (such as one, or two, or three) each independently selected from hydroxyl, thiol, cyano, nitro, oxo, alkylimino, C₁₋₄alkyl, C₁₋₄alkenyl, C₁₋₄alkynyl, C₁₋₄alkoxy, C₁₋₄thioalkyl, C₁₋₄alkenyloxy, C₁₋₄alkynyloxy, halogen, C₁₋₄alkylcarbonyl, carboxy, C₁₋₄alkoxycarbonyl, amino, C₁₋₄alkylamino, di-C₁₋₄alkylamino, C₁₋₄alkylaminocarbonyl, di-C₁₋₄alkylaminocarbonyl, C₁₋₄alkylcarbonylamino, C₁₋₄alkylcarbonyl(C₁₋₄alkyl)amino, sulfonyl, sulfamoyl, alkylsulfamoyl, C₁₋₄alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁₋₄alkoxy groups.

When a heterocycloalkyl forms part of other groups like “heterocycloalkyl-alkyl”, “heterocycloalkoxy”, “heterocycloalkyl-aryl”, the heteroaryl portion shall have the same meaning as described in the above-mentioned definition of “heteroaryl”

“Heterocycloalkyl fused to a phenyl” as used herein, refers to a bicyclic fused ring system that one of the ring is heterocycloalkyl as defined above and the other ring is a phenyl. A heterocycloalkyl fused to a phenyl includes but are not limited to benzo[b][1,4]oxazinyl, oxo-benzo[b][1,4]oxazinyl, tetrahydroquinoxalinyl, tetrahydroquinolinyl, indolinyl, benzo[d]imidazolyl, and the like.

“Hydroxy”, as used herein, refers to the radical —OH.

“Hydroxyalkyl” or “hydroxyl-substituted alkyl” as used herein, refers to an alkyl as defined herein, having one or more of the available hydrogen of the alkyl replaced by a hydroxyl group. For example, a hydroxyC₁₋₄alkyl includes, but are not limited to, —CH₂CH₂OH, —CH(OH)CH₂CH₂OH, —CH(OH)CH₂CH(OH)CH₃.

“Nitro”, as used herein, refers to the radical —NO₂.

“Oxo”, as used herein, refers to the divalent radical ═O

“Protected derivatives” means derivatives of inhibitors in which a reactive site or sites are blocked with protecting groups. Protected derivatives are useful in the preparation of inhibitors or in themselves may be active as inhibitors. Examples of protected group includes, but are not limited to, acetyl, tetrahydropyran, methoxymethyl ether, β-methoxyethoxymethyl ether, p-methoxybenzyl, methylthiomethyl ether, pivaloyl, silyl ether, carbobenzyloxy, benzyl, tert-butoxycarbonyl, ρ-methoxyphenyl, 9-fluorenylmethyloxycarbonyl, acetals, ketals, acylals, dithianes, methylesters, benzyl esters, tert-butyl esters, and silyl esters. A comprehensive list of suitable protecting groups can be found in T. W. Greene, Protecting Groups in Organic Synthesis, 3rd edition, John Wiley & Sons, Inc. 1999.

“Unsubstituted or substituted” or “optionally substituted” as used herein indicate the substituent bound on the available valance of a named group or radical. “Unsubstituted” as used herein indicates that the named group or radical will have no further non-hydrogen substituents. “Substituted” or “optionally substituted” as used herein indicates that at least one of the available hydrogen atoms of named group or radical has been (or may be) replaced by a non-hydrogen substituent.

“Substituted terminally” as used herein referred to a substituent replacing a hydrogen at a terminal position of the parent molecule. For example C₁₋₄alkyl substituted terminally by an amino means —C₁₋₄alkylene-amino, which includes —(CH₂)—NH₂, —(CH₂)₂—NH₂, —(CH₂)₃—NH₂, —(CH₂)CH₂(CH₂—NH₂), —(CH₂)₄—NH₂, —C(CH₂)(CH₂CH₂—NH₂), —C(CH₃)₂(CH₂—NH₂), and the like.

Unless otherwise specified, examples of substituents may include, but are not limited to, halo, nitro, cyano, thio, oxy, hydroxy, carbonyloxy, C₁₋₆alkoxy, C₆₋₁₀aryloxy, heteroC₅₋₁₀aryloxy, carbonyl, oxycarbonyl, aminocarbonyl, amino, C₁₋₆alkylamino, sulfonamido, imino, sulfonyl, sulfinyl, C₁₋₆alkyl, C₁₋₆haloalkyl, hydroxyC₁₋₆alkyl, carbonylC₁₋₆alkyl, thiocarbonylC₁₋₁₀alkyl, sulfonylC₁₋₆alkyl, sulfinylC₁₋₆alkyl, C₁₋₁₀azaalkyl, iminoC₁₋₆alkyl, C₃₋₁₂cycloalkylC₁₋₆alkyl, C₄₋₁₅heterocycloalkylC₁₋₆alkyl, C₆₋₁₀arylC₁₋₆alkyl, C₅₋₁₀heteroarylC₁₋₆alkyl, C₁₀₋₁₂bicycloarylC₁₋₆alkyl, C₉₋₁₂heterobicycloarylC₁₋₆alkyl, C₃₋₁₂cycloalkyl, C₄₋₁₂heterocycloalkyl, C₉₋₁₂bicycloalkyl, C₃₋₁₂heterobicycloalkyl, C₄₋₁₂aryl, heteroC₁₋₁₀aryl, C₉₋₁₂bicycloaryl and C₄₋₁₂heterobicycloaryl.

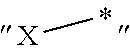

and

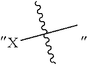

are symbols denoting the point of attachment of X, to other part of the molecule.

Any definition herein may be used in combination with any other definition to describe a composite structural group. By convention, the trailing element of any such definition is that which attaches to the parent moiety. For example, the composite group alkoxyalkyl would represent an alkoxy group attached to the parent molecule through an alkyl group.

It is noted in regard to all of the definitions provided herein that the definitions should be interpreted as being open ended in the sense that further substituents beyond those specified may be included. Hence, a C1 alkyl indicates that there is one carbon atom but does not indicate what are the substituents on the carbon atom. Hence, a C₁alkyl comprises methyl (i.e., —CH₃) as well as —CRₐR_{b}R_{c} where Rₐ, R_{b}, and R_{c} may each independently be hydrogen or any other substituent where the atom attached to the carbon is not a hydrogen atom. Hence, —CF₃, —CH₂OH and —CH₂CN, for example, are all Cialkyls.

### DESCRIPTION OF THE PREFERRED EMBODIMENTS

The invention relates to methods for preventing, inhibiting, ameliorating, or eradicating the pathology and/or symptomology of cryptosporidiosis casued by a protozoans of the genus Cryptosporidium; particularly, Cryptosporidium hominis and Cryptosporidium parvum.

The inventors have discovered selected pyrazolo[1,5-a]pyridines, which are effective in inhibiting the proliferation of Plasmodium parasites (see WO2014/078802), show unexpected inhibitory effect against cryptosporidium species. Selected compounds were effective in mimizing the cytopathic effect of Cyptosporidium infection, reducing the infection rate. The inventors further demonstrated the compounds target phosphatidylinositol-4-OH kinase (PI(4)K), a lipid kinase of the cryptosporidium.

In a first embodiment, the compound for use in the method of the present invention is of Formula I:

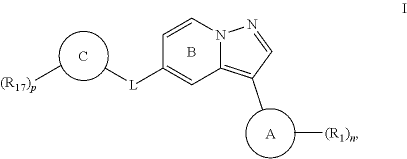

or a pharmaceutical acceptable salt, tautomer or stereoisomer thereof, wherein

n is 0, 1, 2 or 3;

p is 0, 1, 2, or 3;

L is selected from the group consisting of *—(CHR₃)₁₋₃—, *—CHR₃N(R₂)—, CHR₃O—, *—CHR₃S—, *—CHR₃S(O)—, *—CHR₃N(R₂)CHR₃—, *—C(O)—, *—C(O)N(R₂)—, C(O)N(R₂)CHR₃—, *—N(R₂)—, *—N(R₂)CHR₃—, *—N(R₂)C(O)—, *—N(R₂)C(O)N(R₂)—, N(R₂)S(O)₂—, and *—S(O)₂N(R₂)—, wherein
* represents the point of attachment of L to the pyrazolo[1,5-a]pyridine fused ring depicted in Formula I (Ring B);
each R₂ is selected from the group consisting of hydrogen, C₁₋₆alkyl, haloC₁₋₆alkyl, R—C₀₋₄alkylene, and R—C₀₋₄alkylene-C(O)—, wherein R is selected from the group consisting of hydroxyl, C₁₋₄alkoxy, amino, C₁₋₄alkylamino, C₃₋₆cycloalkyl, C₄₋₆heterocycloalkyl, and C₅₋₆heteroaryl, wherein the C₃₋₆cycloalkyl, C₄₋₆heterocycloalkyl, or C₅₋₆heteroaryl of R is unsubstituted or substituted by 1-2 substituents independently selected from the group consisting of halo, amino, hydroxyl, C₁₋₄alkyl, C₁₋₄alkoxy, oxo, and C₅₋₆heteroaryl; and
R₃ is hydrogen or C₁₋₄alkyl;

Ring A is C₆₋₁₀aryl or C₅₋₁₀ heteroaryl;

Ring C is selected from the group consisting of C₆₋₁₀aryl, C₅₋₁₀heteroaryl, C₅₋₇cycloalkyl, C₅₋₇heterocycloalkyl, and a fused bicyclyl comprising a C₅₋₆heterocycloalky fused to a phenyl;

each R₁ is independently selected from the group consisting of halo, cyano, amino, C₁₋₄alkyl, C₁₋₄alkoxy, halo-C₁₋₄alkyl, —C(O)NR₇R₈, —NHC(O)R₁₁, phenyl, C₅₋₆heteroaryl, —C(O)R₁₁, —NHS(O)₂R₁₁, —S(O)₂R₁₁, and —S(O)₂NHR₈, wherein
the phenyl or C₅₋₆heteroaryl of R, is unsubstituted or substituted by 1-2 substituents independently selected from the group consisting of C₁₋₄alkyl, amino, halo, and C₁₋₄alkylamino;
R₇ is selected from the group consisting of hydrogen, C₁₋₄alkyl, and haloC₁₋₄alkyl;
R₈ is selected from the group consisting of hydrogen; haloC₁₋₄alkyl; C₃₋₆cycloalkyl; C₄₋₆heterocycloalkyl; C₁₋₄alkyl unsubstituted or substituted by hydroxy, amino, or C₁₋₄alkylamino; and
R₁₁ is selected from the group consisting of hydroxyl and C₁₋₆alkyl unsubstituted or substituted by 1-2 substituents independently selected from the group consisting of amino, C₃₋₆cycloalkyl, and C₄₋₆heterocycloalkyl;

each R₁₇ is selected from the group consisting of cyano, halo, C₁₋₄alkyl, halo-C₁₋₄alkyl, oxo, C₃₋₆cycloalkyl, —S(O)₂C₁₋₄alkyl; C₁₋₄alkoxy unsubstituted or substituted by hydroxy or amino; and —C(O)R₁₂, wherein R₁₂ is hydrogen, hydroxy or amino.

In a second embodiment, the compound for use in the method of the present invention, with reference to Formula I, wherein

n is 0, 1, 2 or 3;

p is 1 or 2;

L is selected from the group consisting of *—(CHR₃)₁₋₂-, *—CHR₃N(R₂)—, CHR₃O—, *—CHR₃S—, *—CHR₃S(O)—, *—C(O)—, *—C(O)N(R₂)—, *—N(R₂)CHR₃—, *N(R₂)C(O)—, *—N(R₂)C(O)N(R₂)—, *—N(R₂)S(O)₂—, and *—S(O)₂N(R₂)—, wherein
* represents the point of attachment of L to Ring B;
each R₂ is hydrogen, C₁₋₆alkyl or R—C₀₋₄alkylene, wherein R is selected from the group consisting of hydroxyl, C₁₋₄alkoxy, C₁₋₄alkylamino, C₃₋₆cycloalkyl, C₄₋₆heterocycloalkyl, and C₅₋₆heteroaryl, and
R₃ is hydrogen or C₁₋₄alkyl;

Ring A is C₆₋₁₀aryl or C₅₋₁₀heteroaryl;

Ring C is selected from the group consisting of C₆₋₁₀aryl, C₅₋₁₀heteroaryl, C₅₋₇cycloalkyl, and fused bicyclyl comprising a C₅₋₆heterocycloalky fused to a phenyl;

each R₁ is independently selected from the group consisting of halo, cyano, amino, C₁₋₄alkyl, C₁₋₄alkoxy, halo-C₁₋₄alkyl, —C(O)NR₇R₈, —NHC(O)R₁₁, C₅₋₆heteroaryl; —C(O)R₁₁, —NHS(O)₂R₁₁, —S(O)₂R₁₁, and —S(O)₂NHR₈, wherein
the C₅₋₆heteroaryl of R₁ is unsubstituted or substituted by C₁₋₄alkylamino;
R₇ is hydrogen or C₁₋₄alkyl;
R₈ is selected from hydrogen; hydroxy; C₃₋₆cycloalkyl; C₄₋₆heterocycloalkyl; C₁₋₄alkyl unsubstituted or substituted by hydroxy, amino or C₁₋₄alkylamino; and
R₁₁ is hydroxy or C₁₋₆alkyl unsubstituted or substituted by 1-2 substituents independently selected from amino and C₃₋₆cycloalkyl; and

each R₁₇ is independently selected from cyano; halo; C₁₋₄alkyl; halo-C₁₋₄alkyl; oxo; C₃₋₆cycloalkyl; —S(O)₂C₁₋₄alkyl; C₁₋₄alkoxy unsubstituted or substituted by either hydroxyl or amino; and —C(O)R₁₂ wherein R₁₂ is hydrogen, hydroxy or amino.

In a third embodiment, the compound for use in the method of the present invention, with reference to Formula I, wherein

n is 0, 1, 2 or 3;

p is 0, 1, 2 or 3;

L is selected from *—(CHR₃)₁₋₃—, *—CHR₃N(R₂)—, *—CHR₃O—, *—CHR₃S—, *—CHR₃S(O)—, *—CHR₃N(R₂)CHR₃—, *—C(O)—, *—C(O)N(R₂)—, *—C(O)N(R₂)CHR₃—, N(R₂)—, *—N(R₂)CHR₃—, *—N(R₂)C(O)—, *—N(R₂)C(O)N(R₂)—, and *—N(R₂)S(O)₂—, wherein
* represents the point of attachment of L to the pyrazolo[1,5-a]pyridine fused ring depicted in Formula I;
each R₂ is independently selected from the group consisting of hydrogen, C₁₋₆alkyl, haloC₁₋₆alkyl, R—C₀₋₄alkylene, and R—C₀₋₄alkylene-C(O)—, wherein R is selected from the group consisting of hydroxyl, C₁₋₄alkoxy, amino, C₁₋₄alkylamino, C₃₋₆cycloalkyl, C₄₋₆heterocycloalkyl, and C₅₋₆heteroaryl, wherein the C₃₋₆cycloalkyl, C₄₋₆heterocycloalkyl, and C₅₋₆heteroaryl of R are each unsubstituted or substituted by 1-2 substituents independently selected from the group consisting of halo, amino, hydroxyl, C₁₋₄alkyl, C₁₋₄alkoxy, oxo, and C₅₋₆heteroaryl; and
each R₃ is independently selected from the group consisting of hydrogen and C₁₋₄alkyl;

Ring A is selected from the group consisting of C₆₋₁₀aryl and C₅₋₁₀heteroaryl;

Ring C is selected from the group consisting of C₆₋₁₀aryl, C₅₋₁₀heteroaryl, C₅₋₇cycloalkyl, C₅₋₇heterocycloalkyl, and fused bicyclyl comprising a C₅₋₆heterocycloalky fused to a phenyl;

each R₁ is independently selected from the group consisting of halo, cyano, amino, C₁₋₄alkyl, C₁₋₄alkoxyl, halo-C₁₋₄alkyl, —C(O)NR₇R₈, —NHC(O)R₁₁, phenyl, and C₅₋₆heteroaryl; wherein
the phenyl and C₅₋₆heteroaryl of R, are each unsubstituted or substituted by 1-2 substituents independently selected from the group consisting of C₁₋₄alkyl, amino, halo, and C₁₋₄alkylamino;
R₇ and R₈ are each independently selected from hydrogen, C₁₋₄alkyl and haloC₁₋₄alkyl;
R₁₁ is C₁₋₆alkyl, unsubstituted or substituted by 1-2 substituents independently selected from the group consisting of amino, C₃₋₆cycloalkyl and C₄₋₆heterocycloalkyl;

R₁₇ is selected from the group consisting of cyano, halo, C₁₋₄alkyl, halo-C₁ 4alkyl, oxo, C₃₋₆cycloalkyl, and —SO₂—C₁₋₄alkyl.

In one embodiment of the first, second and third embodiments for the compounds for use in the method of the present invention, in reference to Formula I, L is selected from the group consisting of *—(CHR₃)—, *—CHR₃N(R₂)—, *—C(O)—, *—C(O)N(R₂)—, *—N(R₂)C(O)—, and *—S(O)₂N(R₂)—, wherein
* represents the point of attachment of L to Ring B;
R₂ is hydrogen, C₁₋₆alkyl or R—C₀₋₄alkylene, wherein R is selected from the group consisting of C₁₋₄alkylamino, C₃₋₆cycloalkyl, C₄₋₆heterocycloalkyl, and C₅₋₆heteroaryl; and
R₃ is C₁₋₄alkyl.

In one variation, L is selected from the group consisting of *—CHR₃—, CHR₃N(R₂)—, *—CHR₃O—, *—CHR₃S—, *—CHR₃S(O)—, *—C(O)—, *—C(O)N(R₂)—, *—N(R₂)—, *—N(R₂)CHR₃—, *—N(R₂)C(O)—, *—N(R₂)C(O)N(R₂)—, and *—N(R₂)S(O)₂—, wherein
each R₂ is independently hydrogen, C₁₋₆alkyl or R—C₀₋₄alkylene, wherein R is selected from the group consisting of C₁₋₄alkoxy, C₁₋₄alkylamino, di-C₁₋₄alkylamino, C₃₋₆cycloalkyl, C₄₋₆heterocycloalkyl and C₅₋₆heteroaryl, wherein the C₃₋₆cycloalkyl, C₄₋₆heterocycloalkyl or C₅₋₆heteroaryl of R is unsubstituted or substituted by 1-2 substituents independently selected from the group consisting of halo, amino, hydroxyl, C₁₋₄alkyl, C₁₋₄alkoxy, oxo, and C₅₋₆heteroaryl.

In another variation, L is *—C(O)N(R₂)— or *—N(R₂)C(O)—, wherein R₂ is hydrogen, C₁₋₄alkyl or R—C₀₋₄alkylene, wherein R is selected from the group consisting of C₁₋₄alkylamino, C₃₋₆cycloalkyl, C₄₋₆heterocycloalkyl, and C₅₋₆heteroaryl, each of which is unsubstituted or substituted by 1-2 substituents independently selected from the group consisting of halo, amino, hydroxyl, C₁₋₄alkyl, C₁₋₄alkoxy, oxo, and C₅₋₆heteroaryl.

In still another variation, L is *—(CHR₃)— or *—C(O)N(R₂)—, wherein * represents the point of attachment of L to Ring B; R₂ is CO₁₆alkyl or C₃₋₆cycloalkyl; and R₃ is C₁₋₄alkyl. In still another variation, L is *—C(O)N(R₂)—, wherein * represents the point of attachment of L to Ring B and R₂ is C₁₋₆alkyl or C₃₋₆cycloalkyl. In yet another variation, L is *—(CHR₃)—, wherein * represents the point of attachment of L to Ring B and R₃ is-C₁₋₄alkyl. In yet another variation L is *—C(O)— or *—CH(CH₃)—,

In another embodiment of the compounds for use in the method of the present invention, in reference to the first, second and third embodiments and the above variations, and Formula I, Ring A is selected from the group consisting of phenyl, pyridinyl, pyrimidinyl, pyrrolopyridinyl, and indazolyl. In one variation, Ring A is selected from the group consisting of

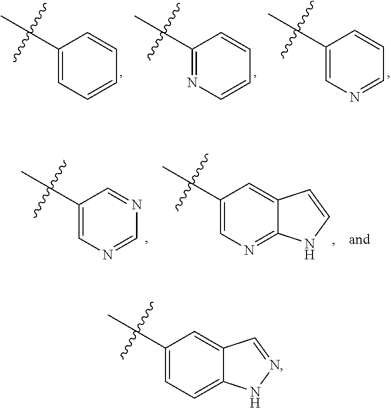 [SMILES: *c1ccccc1 ; *c1ccccn1 ; *c1cccnc1 ; *c1cncnc1 ; *c1cnc2[nH]ccc2c1 ; *c1ccc2[nH]ncc2c1]

each of which is unsubstituted or substituted by (R₁)ₙ.

In another embodiment of the compounds for use in the method of the present invention, in reference to the first, second and third embodiments and the above embodiments and variations, and Formula I, Ring C is selected from the group consisting of phenyl, pyridinyl, cyclohexyl, and dihydrobenzooxazinyl. In one variation, Ring C is selected from the group consisting of

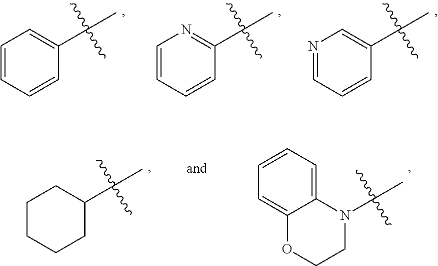 [SMILES: *c1ccccc1 ; *c1ccccn1 ; *c1cccnc1 ; *C1CCCCC1 ; *N1CCOc2ccccc21]

each of which is unsubstituted or substituted by (R₁₇)ₚ.

In still another embodiment of the method of the present invention, with reference to any one of the above embodiments and variations, each R₁ is independently selected from the group consisting of halo, cyano, amino, C₁₋₄alkyl, C₁₋₄alkoxy, halo-C₁₋₄alkyl, —C(O)NR₇R₈, and —NHC(O)R₁₁, wherein
R₇ and R₈ are independently hydrogen or C₁₋₄alkyl;
R₁₁ is C₁₋₆alkyl unsubstituted or substituted by 1-2 substituents independently selected from the group consisting of amino and C₃₋₆cycloalkyl.

In one variation, each R, is independently selected from the group consisting of halo, cyano, methyl, trifluoromethyl, —NH₂, —C(O)NH₂, —C(O)NH(CH₃), —C(O)NHCH₂CH₃, —C(O)N(CH₃)₂, —NHC(O)CH₃, —NHC(O)CH₂NH₂, —NHC(O)(CH₂)₂OH, —NHC(O)CH(NH₂)(CH₃), —NHC(O)CH(NH₂)CH(CH₃)₂, —NHC(O)CH(CH₃)₂.

In another variation, each R₁ is independently selected from the group consisting of methyl, —NH₂, —C(O)NH₂, —C(O)NH(CH₃), and NHC(O)CH(NH₂)(CH₃). In another variation, R₁ is trifluoromethyl. In another variation, R, is —NH₂. In still another variation, R, is —C(O)NH₂. In yet another variation, R, is —C(O)NHCH₃. In yet another variation, R, is —C(O)N(CH₃)₂. In still yet another variation R, is NH₂.

In yet another embodiment of the method of the invention, with reference to any one of the above embodiments and variations, each R₁₇ is independently selected from the group consisting of cyano, halo, C₁₋₄alkyl, halo-C₁₋₄alkyl, oxo, C₁₋₄alkoxy, and —C(O)H.

In one variation, each R₁₇ is independently selected from the group consisting of cyano, fluoro, chloro, methyl, trifluoromethyl, methoxy, oxo and —C(O)H. In another variation, each R₁₇ is independently halo, oxo or —C(O)H. In another variation, each R₁₇ is independently selected from methyl, methoxy, cyano, and halo. In still another variation, R₁₇ is cyano. In yet another variation, R₁₇ is halo. In still another variation, R₁₇ is trifluoromethyl.

In a particular embodiment of the method of the invention, the compound is of Formula Ia:

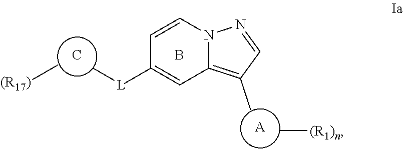

or a pharmaceutical acceptable salt, tautomer or stereoisomer thereof, wherein

n is 0 or 1;

p is 1 or 2;

L is *—CHR₃— or *—C(O)NR₂—; wherein
* represents the point of attachment of L to Ring B;
R₂ is C₁₋₄alkyl or C₃₋₆cycloalkyl; and
R₃ is C₁₋₄alkyl;

Ring A is phenyl or C₅₋₁₀heteroaryl;

Ring C is phenyl, C₅₋₁₀heteroaryl or fused bicyclyl comprising a C₅₋₆heterocycloalky fused to a phenyl;

each R₁ is independently C₁₋₄alkyl, —NHC(O)R₁₁, or —C(O)NR₇R⁸, wherein
R₇ and R₈ is independently hydrogen or C₁₋₄alkyl;
R₁₁ is C₁₋₄alkyl substituted by —NH₂; and

each R₁₇ is independently selected from the group consisting of halo, cyano, C₁₋₄alkyl, haloC₁₋₄alkyl, and C₁₋₄alkoxy.

In another embodiment, in reference to Formula Ia,

L is *—CHCH₃—, *—C(O)N(CH₃)—, *—C(O)NCH(CH₃)₂—, *—C(O)N(cyclopropyl)-, or *—C(O)N(cyclobutyl)-;

Ring A is selected from the group consisting of phenyl, pyridinyl, pyrrolopyridinyl, and indazolyl,

Ring C is phenyl, pyridinyl or dihydrobenzooxazinyl;

each R₁ is independently selected from the group consisting of methyl, —C(O)NH₂, —C(O)NHCH₃, or —NHC(O)CH(NH₂)CH₃; and

each R₁₇ is independently selected from the group consisting of cyano, fluoro, chloro, methyl, trifluoromethyl, methoxy, and oxo.

In one variation of the method of the present invention, with reference to the particular embodiment above, L is *—CHCH₃—. In another variation, L is *—C(O)N(CH₃)—. In yet another variation, L is *—C(O)NCH(CH₃)₂—. In still yet another variation, L is *—C(O)N(cyclopropyl)-. In still yet another variation, L is *—C(O)N(cyclobutyl)-.

In another variation of the method of the present invention, Ring A is

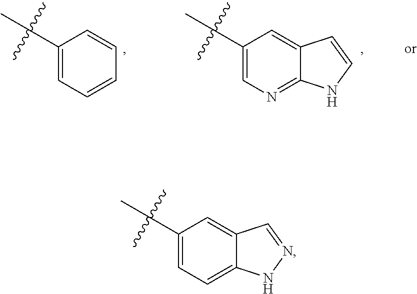 [SMILES: *c1ccccc1 ; *c1cnc2[nH]ccc2c1 ; *c1ccc2[nH]ncc2c1]

each of which is unsubstituted or substituted by R₁. In another variation, Ring A is

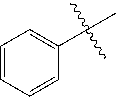 [SMILES: *c1ccccc1]

unsubstituted or substituted by R₁. In still another variation, Ring A is

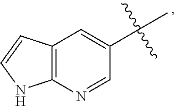 [SMILES: *c1cnc2[nH]ccc2c1]

unsubstituted or substituted by R₁. In still yet another variation, Ring A is

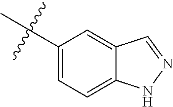 [SMILES: *c1ccc2[nH]ncc2c1]

unsubstituted or substituted by R₁.

In one embodiment of the method of the invention, in reference to Formula Ia and the first and second particular embodiment, Ring C is selected from the group consisting of

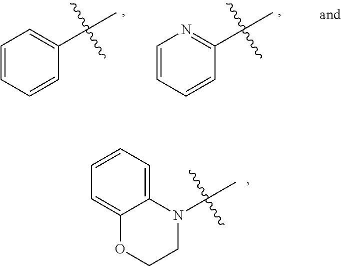 [SMILES: *c1ccccc1 ; *c1ccccn1 ; *N1CCOc2ccccc21]

each of which is unsubstituted or substituted by (R₁₇)ₚ. In one variation, Ring C is

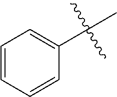 [SMILES: *c1ccccc1]

substituted by R₁₇. In another variation, Ring C is

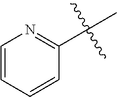 [SMILES: *c1ccccn1]

substituted by (R₁₇)₁₋₂. In another variation, Ring C is

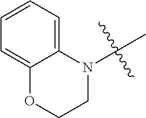 [SMILES: *N1CCOc2ccccc21]

substituted by (R₁₇)₁₋₂.

In still another embodiment of the method of the present invention, with reference to the particular embodiment or any one of the variations above, R, is methyl. In one variation, R₁ is —C(O)NH₂. In another variation, R, is —C(O)NHCH₃. In still another variation, R, is —NHC(O)CH(NH₂)CH₃.

In still another variation of the compounds of the present invention, with reference to the particular embodiments or any one of the variations above, each R₁₇ is independently halo, cyano, methoxy, or oxo. In another variation, R₁₇ is cyano. In still another variation, R₁₇ is trifluoromethyl. In yet another variation R₁₇ is methyl. In another variation R₁₇ is halo.

Particular compound or a pharmaceutically acceptable salt, tautomer or stereoisomer thereof, useful in the method of the current invention is selected from Table I below:

TABLE I
Listing of Compounds

| | |
|---|---|
| 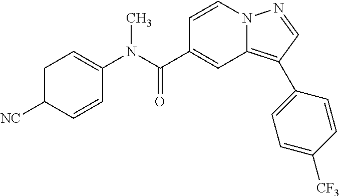 [SMILES: CN(C(=O)c1ccn2ncc(-c3ccc(C(F)(F)F)cc3)c2c1)C1=CCC(C#N)C=C1] | 1 |
| 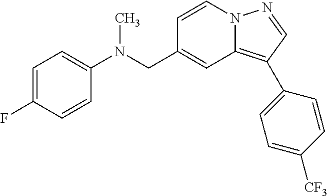 [SMILES: CN(Cc1ccn2ncc(-c3ccc(C(F)(F)F)cc3)c2c1)c1ccc(F)cc1] | 2 |
| 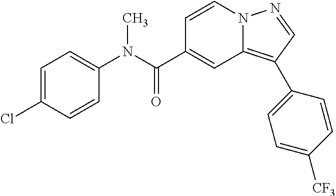 [SMILES: CN(C(=O)c1ccn2ncc(-c3ccc(C(F)(F)F)cc3)c2c1)c1ccc(Cl)cc1] | 3 |
| 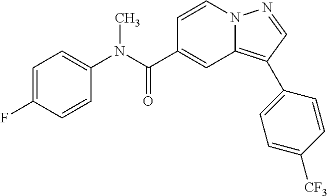 [SMILES: CN(C(=O)c1ccn2ncc(-c3ccc(C(F)(F)F)cc3)c2c1)c1ccc(F)cc1] | 4 |
| 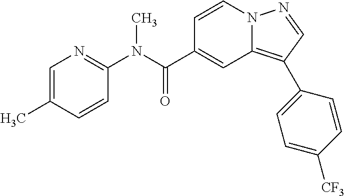 [SMILES: Cc1ccc(N(C)C(=O)c2ccn3ncc(-c4ccc(C(F)(F)F)cc4)c3c2)nc1] | 5 |
| 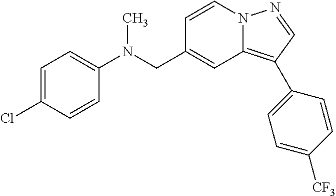 [SMILES: CN(Cc1ccn2ncc(-c3ccc(C(F)(F)F)cc3)c2c1)c1ccc(Cl)cc1] | 6 |
| 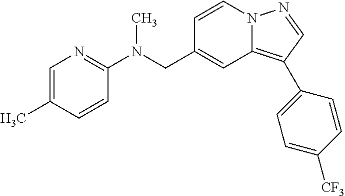 [SMILES: Cc1ccc(N(C)Cc2ccn3ncc(-c4ccc(C(F)(F)F)cc4)c3c2)nc1] | 7 |
| 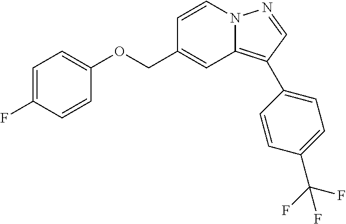 [SMILES: Fc1ccc(OCc2ccn3ncc(-c4ccc(C(F)(F)F)cc4)c3c2)cc1] | 8 |
| 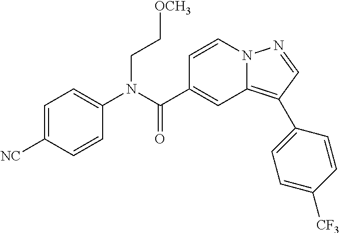 [SMILES: COCCN(C(=O)c1ccn2ncc(-c3ccc(C(F)(F)F)cc3)c2c1)c1ccc(C#N)cc1] | 9 |
| 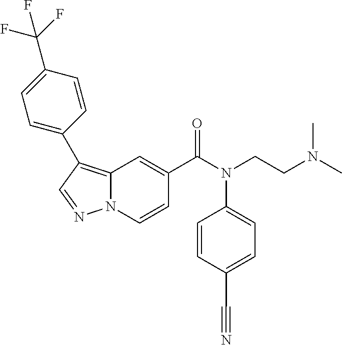 [SMILES: CN(C)CCN(C(=O)c1ccn2ncc(-c3ccc(C(F)(F)F)cc3)c2c1)c1ccc(C#N)cc1] | 10 |
| 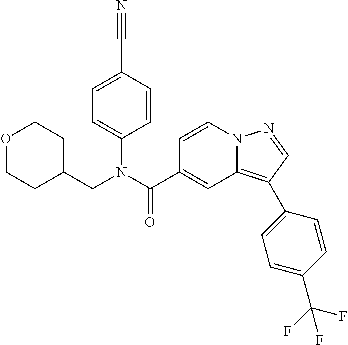 [SMILES: N#Cc1ccc(N(CC2CCOCC2)C(=O)c2ccn3ncc(-c4ccc(C(F)(F)F)cc4)c3c2)cc1] | 11 |
| 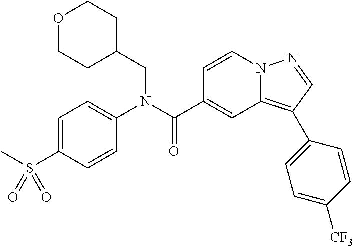 [SMILES: CS(=O)(=O)c1ccc(N(CC2CCOCC2)C(=O)c2ccn3ncc(-c4ccc(C(F)(F)F)cc4)c3c2)cc1] | 12 |
| 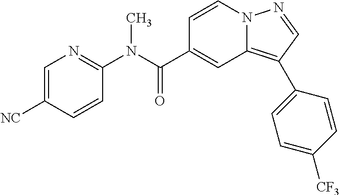 [SMILES: CN(C(=O)c1ccn2ncc(-c3ccc(C(F)(F)F)cc3)c2c1)c1ccc(C#N)cn1] | 13 |
| 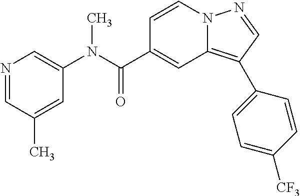 [SMILES: Cc1cncc(N(C)C(=O)c2ccn3ncc(-c4ccc(C(F)(F)F)cc4)c3c2)c1] | 14 |
| 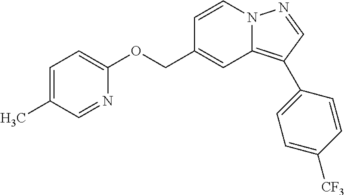 [SMILES: Cc1ccc(OCc2ccn3ncc(-c4ccc(C(F)(F)F)cc4)c3c2)nc1] | 15 |
| 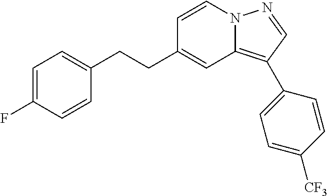 [SMILES: Fc1ccc(CCc2ccn3ncc(-c4ccc(C(F)(F)F)cc4)c3c2)cc1] | 16 |
| 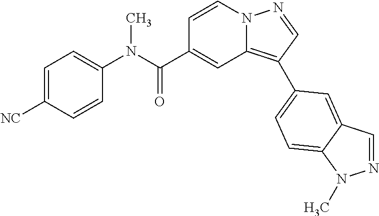 [SMILES: CN(C(=O)c1ccn2ncc(-c3ccc4c(cnn4C)c3)c2c1)c1ccc(C#N)cc1] | 17 |
| 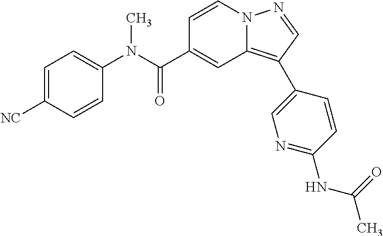 [SMILES: CC(=O)Nc1ccc(-c2cnn3ccc(C(=O)N(C)c4ccc(C#N)cc4)cc23)cn1] | 18 |
| 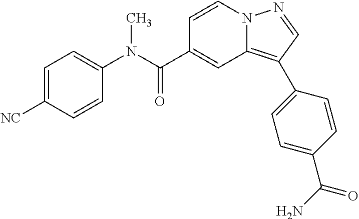 [SMILES: CN(C(=O)c1ccn2ncc(-c3ccc(C(N)=O)cc3)c2c1)c1ccc(C#N)cc1] | 19 |
| 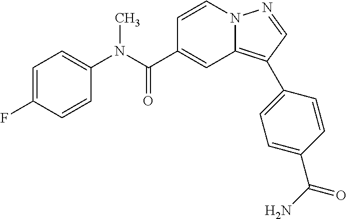 [SMILES: CN(C(=O)c1ccn2ncc(-c3ccc(C(N)=O)cc3)c2c1)c1ccc(F)cc1] | 20 |
| 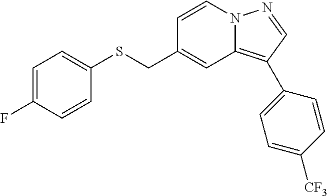 [SMILES: Fc1ccc(SCc2ccn3ncc(-c4ccc(C(F)(F)F)cc4)c3c2)cc1] | 21 |
| 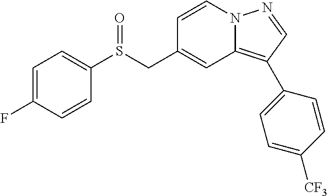 [SMILES: O=S(Cc1ccn2ncc(-c3ccc(C(F)(F)F)cc3)c2c1)c1ccc(F)cc1] | 22 |
| 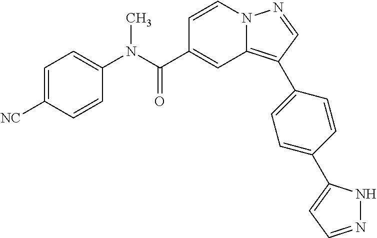 [SMILES: CN(C(=O)c1ccn2ncc(-c3ccc(-c4ccn[nH]4)cc3)c2c1)c1ccc(C#N)cc1] | 23 |
| 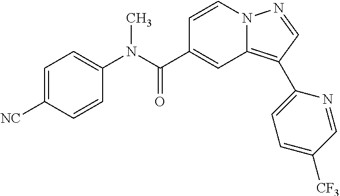 [SMILES: CN(C(=O)c1ccn2ncc(-c3ccc(C(F)(F)F)cn3)c2c1)c1ccc(C#N)cc1] | 24 |
| 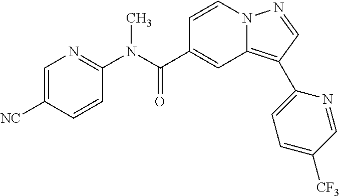 [SMILES: CN(C(=O)c1ccn2ncc(-c3ccc(C(F)(F)F)cn3)c2c1)c1ccc(C#N)cn1] | 25 |
| 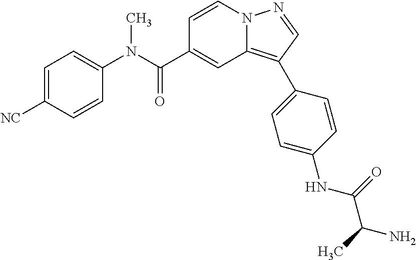 [SMILES: C[C@H](N)C(=O)Nc1ccc(-c2cnn3ccc(C(=O)N(C)c4ccc(C#N)cc4)cc23)cc1] | 26 |
| 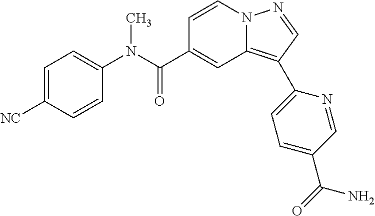 [SMILES: CN(C(=O)c1ccn2ncc(-c3ccc(C(N)=O)cn3)c2c1)c1ccc(C#N)cc1] | 27 |
| 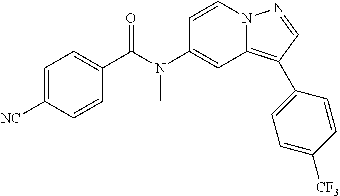 [SMILES: CN(C(=O)c1ccc(C#N)cc1)c1ccn2ncc(-c3ccc(C(F)(F)F)cc3)c2c1] | 28 |
| 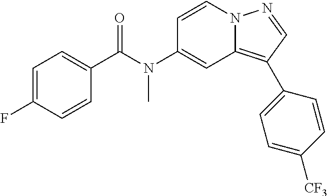 [SMILES: CN(C(=O)c1ccc(F)cc1)c1ccn2ncc(-c3ccc(C(F)(F)F)cc3)c2c1] | 29 |
| 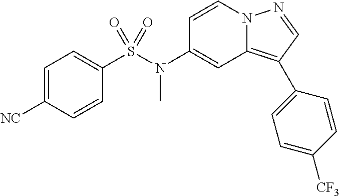 [SMILES: CN(c1ccn2ncc(-c3ccc(C(F)(F)F)cc3)c2c1)S(=O)(=O)c1ccc(C#N)cc1] | 30 |
| 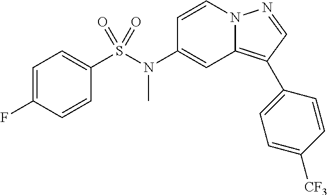 [SMILES: CN(c1ccn2ncc(-c3ccc(C(F)(F)F)cc3)c2c1)S(=O)(=O)c1ccc(F)cc1] | 31 |
| 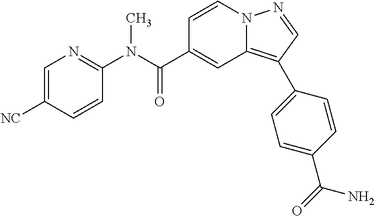 [SMILES: CN(C(=O)c1ccn2ncc(-c3ccc(C(N)=O)cc3)c2c1)c1ccc(C#N)cn1] | 32 |
| 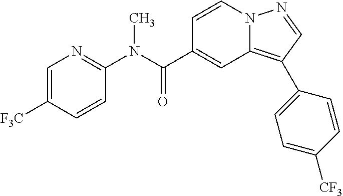 [SMILES: CN(C(=O)c1ccn2ncc(-c3ccc(C(F)(F)F)cc3)c2c1)c1ccc(C(F)(F)F)cn1] | 33 |
| 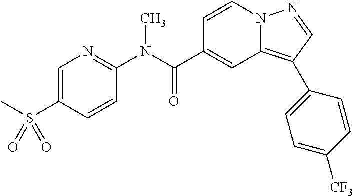 [SMILES: CN(C(=O)c1ccn2ncc(-c3ccc(C(F)(F)F)cc3)c2c1)c1ccc(S(C)(=O)=O)cn1] | 34 |
| 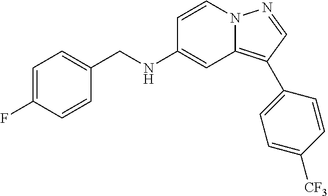 [SMILES: Fc1ccc(CNc2ccn3ncc(-c4ccc(C(F)(F)F)cc4)c3c2)cc1] | 35 |
| 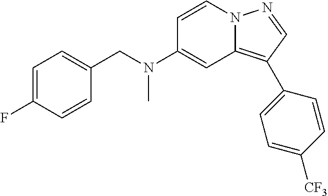 [SMILES: CN(Cc1ccc(F)cc1)c1ccn2ncc(-c3ccc(C(F)(F)F)cc3)c2c1] | 36 |
| 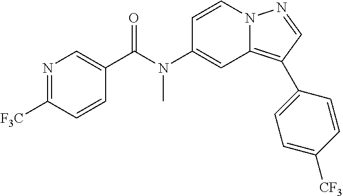 [SMILES: CN(C(=O)c1ccc(C(F)(F)F)nc1)c1ccn2ncc(-c3ccc(C(F)(F)F)cc3)c2c1] | 37 |
| 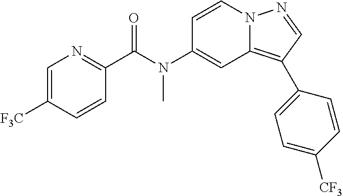 [SMILES: CN(C(=O)c1ccc(C(F)(F)F)cn1)c1ccn2ncc(-c3ccc(C(F)(F)F)cc3)c2c1] | 38 |
| 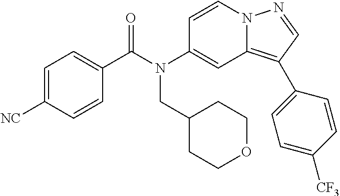 [SMILES: N#Cc1ccc(C(=O)N(CC2CCOCC2)c2ccn3ncc(-c4ccc(C(F)(F)F)cc4)c3c2)cc1] | 39 |
| 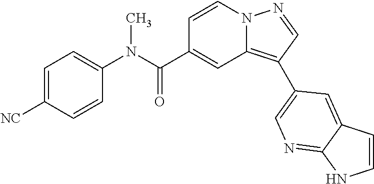 [SMILES: CN(C(=O)c1ccn2ncc(-c3cnc4[nH]ccc4c3)c2c1)c1ccc(C#N)cc1] | 40 |
| 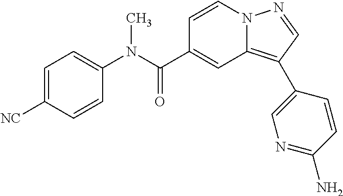 [SMILES: CN(C(=O)c1ccn2ncc(-c3ccc(N)nc3)c2c1)c1ccc(C#N)cc1] | 41 |
| 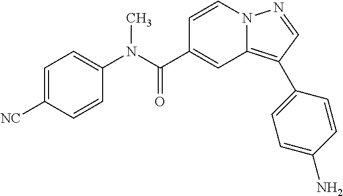 [SMILES: CN(C(=O)c1ccn2ncc(-c3ccc(N)cc3)c2c1)c1ccc(C#N)cc1] | 42 |
| 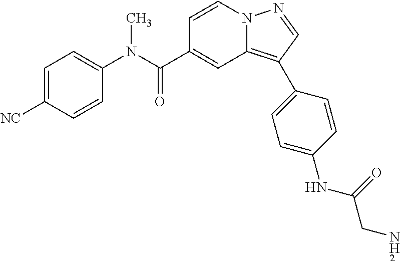 [SMILES: CN(C(=O)c1ccn2ncc(-c3ccc(NC(=O)CN)cc3)c2c1)c1ccc(C#N)cc1] | 43 |
| 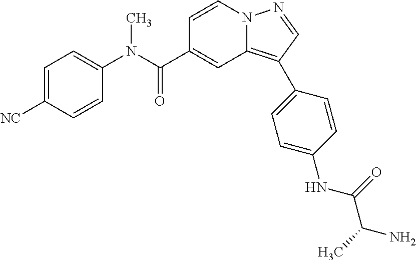 [SMILES: C[C@@H](N)C(=O)Nc1ccc(-c2cnn3ccc(C(=O)N(C)c4ccc(C#N)cc4)cc23)cc1] | 44 |
| 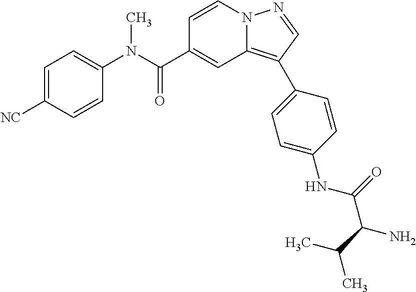 [SMILES: CC(C)[C@H](N)C(=O)Nc1ccc(-c2cnn3ccc(C(=O)N(C)c4ccc(C#N)cc4)cc23)cc1] | 45 |
| 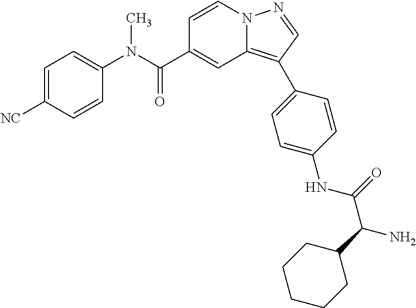 [SMILES: CN(C(=O)c1ccn2ncc(-c3ccc(NC(=O)[C@@H](N)C4CCCCC4)cc3)c2c1)c1ccc(C#N)cc1] | 46 |
| 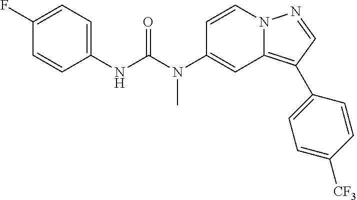 [SMILES: CN(C(=O)Nc1ccc(F)cc1)c1ccn2ncc(-c3ccc(C(F)(F)F)cc3)c2c1] | 47 |
| 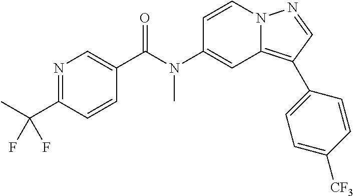 [SMILES: CN(C(=O)c1ccc(C(C)(F)F)nc1)c1ccn2ncc(-c3ccc(C(F)(F)F)cc3)c2c1] | 48 |
| 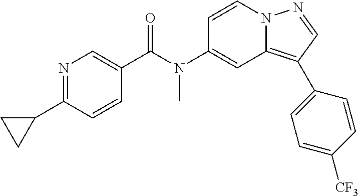 [SMILES: CN(C(=O)c1ccc(C2CC2)nc1)c1ccn2ncc(-c3ccc(C(F)(F)F)cc3)c2c1] | 49 |
| 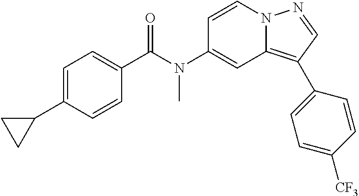 [SMILES: CN(C(=O)c1ccc(C2CC2)cc1)c1ccn2ncc(-c3ccc(C(F)(F)F)cc3)c2c1] | 50 |
| 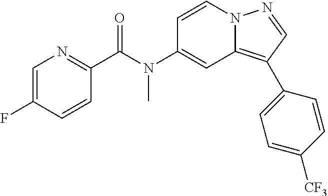 [SMILES: CN(C(=O)c1ccc(F)cn1)c1ccn2ncc(-c3ccc(C(F)(F)F)cc3)c2c1] | 51 |
| 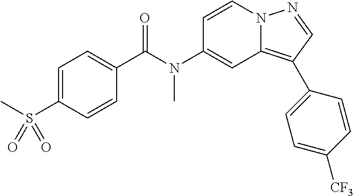 [SMILES: CN(C(=O)c1ccc(S(C)(=O)=O)cc1)c1ccn2ncc(-c3ccc(C(F)(F)F)cc3)c2c1] | 52 |
| 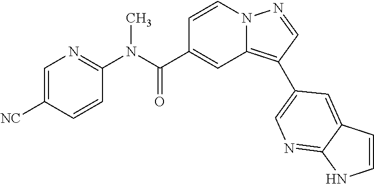 [SMILES: CN(C(=O)c1ccn2ncc(-c3cnc4[nH]ccc4c3)c2c1)c1ccc(C#N)cn1] | 53 |
| 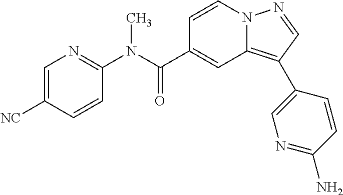 [SMILES: CN(C(=O)c1ccn2ncc(-c3ccc(N)nc3)c2c1)c1ccc(C#N)cn1] | 54 |
| 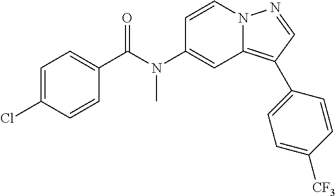 [SMILES: CN(C(=O)c1ccc(Cl)cc1)c1ccn2ncc(-c3ccc(C(F)(F)F)cc3)c2c1] | 55 |
| 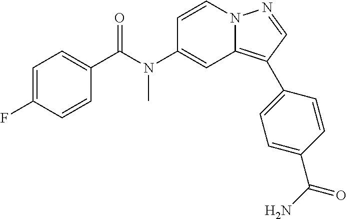 [SMILES: CN(C(=O)c1ccc(F)cc1)c1ccn2ncc(-c3ccc(C(N)=O)cc3)c2c1] | 56 |
| 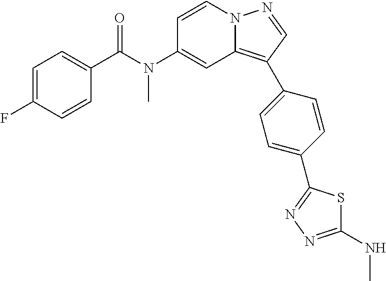 [SMILES: CNc1nnc(-c2ccc(-c3cnn4ccc(N(C)C(=O)c5ccc(F)cc5)cc34)cc2)s1] | 57 |
| 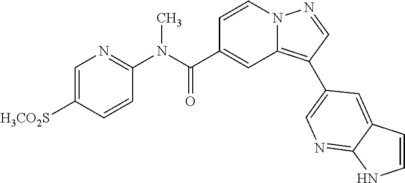 | 58 |
| 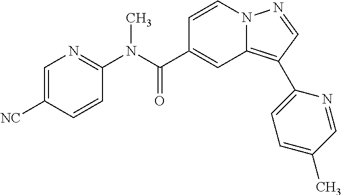 [SMILES: Cc1ccc(-c2cnn3ccc(C(=O)N(C)c4ccc(C#N)cn4)cc23)nc1] | 59 |
| 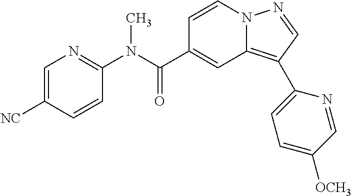 [SMILES: COc1ccc(-c2cnn3ccc(C(=O)N(C)c4ccc(C#N)cn4)cc23)nc1] | 60 |
| 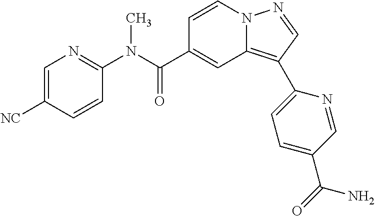 [SMILES: CN(C(=O)c1ccn2ncc(-c3ccc(C(N)=O)cn3)c2c1)c1ccc(C#N)cn1] | 61 |
| 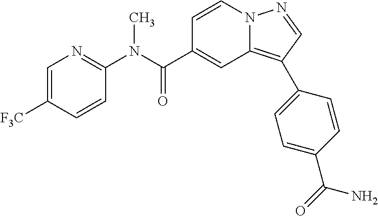 [SMILES: CN(C(=O)c1ccn2ncc(-c3ccc(C(N)=O)cc3)c2c1)c1ccc(C(F)(F)F)cn1] | 62 |
| 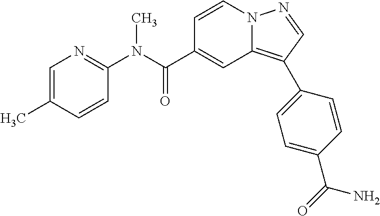 [SMILES: Cc1ccc(N(C)C(=O)c2ccn3ncc(-c4ccc(C(N)=O)cc4)c3c2)nc1] | 63 |
| 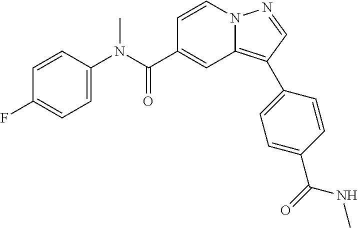 [SMILES: CNC(=O)c1ccc(-c2cnn3ccc(C(=O)N(C)c4ccc(F)cc4)cc23)cc1] | 64 |
| 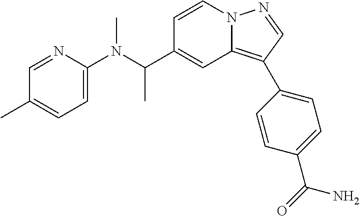 [SMILES: Cc1ccc(N(C)C(C)c2ccn3ncc(-c4ccc(C(N)=O)cc4)c3c2)nc1] | 65 |
| 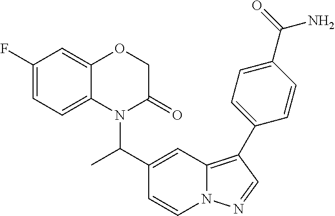 [SMILES: CC(c1ccn2ncc(-c3ccc(C(N)=O)cc3)c2c1)N1C(=O)COc2cc(F)ccc21] | 66 |
| 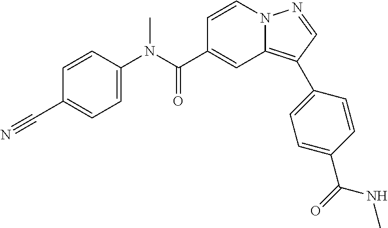 [SMILES: CNC(=O)c1ccc(-c2cnn3ccc(C(=O)N(C)c4ccc(C#N)cc4)cc23)cc1] | 67 |
| 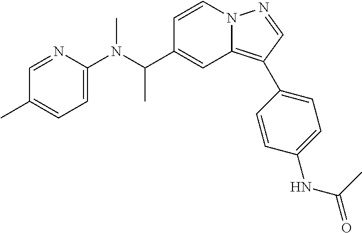 [SMILES: CC(=O)Nc1ccc(-c2cnn3ccc(C(C)N(C)c4ccc(C)cn4)cc23)cc1] | 68 |
| 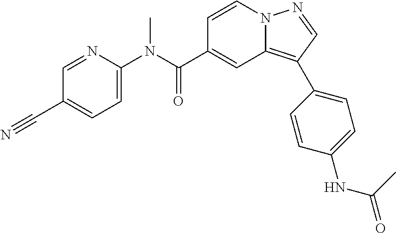 [SMILES: CC(=O)Nc1ccc(-c2cnn3ccc(C(=O)N(C)c4ccc(C#N)cn4)cc23)cc1] | 69 |
| 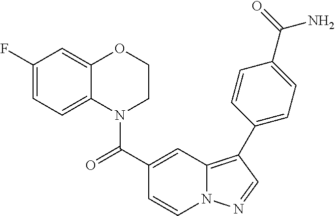 [SMILES: NC(=O)c1ccc(-c2cnn3ccc(C(=O)N4CCOc5cc(F)ccc54)cc23)cc1] | 71 |
| 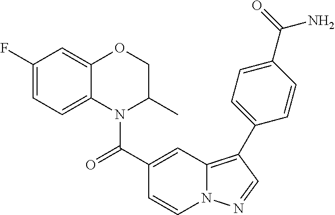 [SMILES: CC1COc2cc(F)ccc2N1C(=O)c1ccn2ncc(-c3ccc(C(N)=O)cc3)c2c1] | 72 |
| 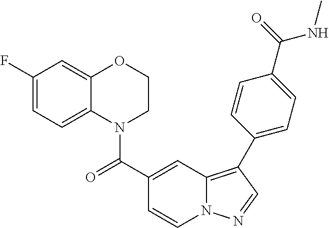 [SMILES: CNC(=O)c1ccc(-c2cnn3ccc(C(=O)N4CCOc5cc(F)ccc54)cc23)cc1] | 73 |
| 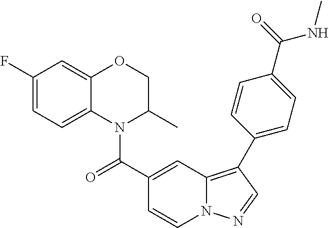 [SMILES: CNC(=O)c1ccc(-c2cnn3ccc(C(=O)N4c5ccc(F)cc5OCC4C)cc23)cc1] | 74 |
| 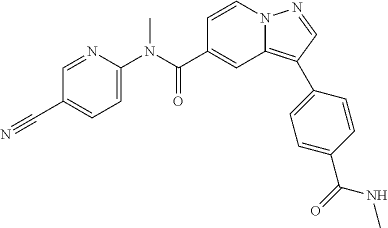 [SMILES: CNC(=O)c1ccc(-c2cnn3ccc(C(=O)N(C)c4ccc(C#N)cn4)cc23)cc1] | 75 |
| 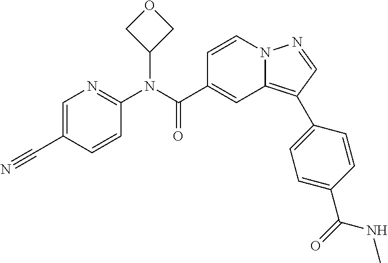 [SMILES: CNC(=O)c1ccc(-c2cnn3ccc(C(=O)N(c4ccc(C#N)cn4)C4COC4)cc23)cc1] | 76 |
| 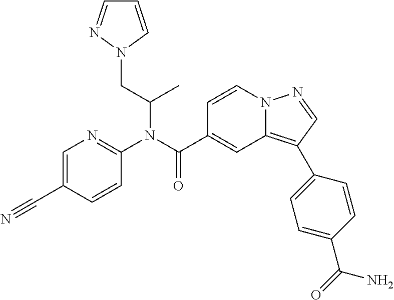 [SMILES: CC(Cn1cccn1)N(C(=O)c1ccn2ncc(-c3ccc(C(N)=O)cc3)c2c1)c1ccc(C#N)cn1] | 77 |
| 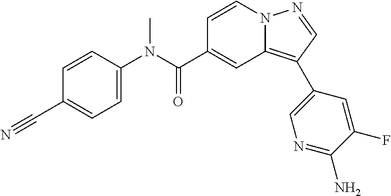 [SMILES: CN(C(=O)c1ccn2ncc(-c3cnc(N)c(F)c3)c2c1)c1ccc(C#N)cc1] | 79 |
| 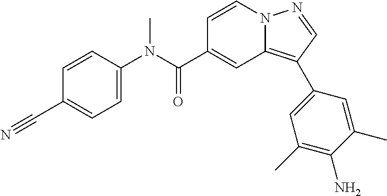 [SMILES: Cc1cc(-c2cnn3ccc(C(=O)N(C)c4ccc(C#N)cc4)cc23)cc(C)c1N] | 80 |
| 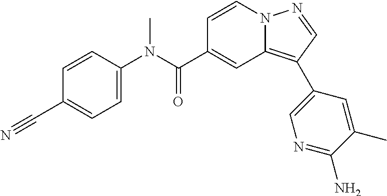 [SMILES: Cc1cc(-c2cnn3ccc(C(=O)N(C)c4ccc(C#N)cc4)cc23)cnc1N] | 81 |
| 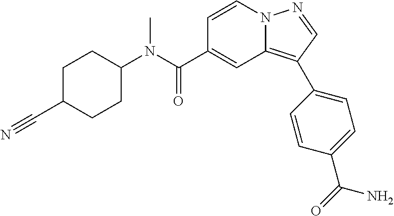 [SMILES: CN(C(=O)c1ccn2ncc(-c3ccc(C(N)=O)cc3)c2c1)C1CCC(C#N)CC1] | 82 |
| 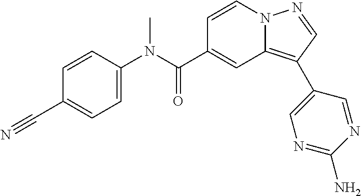 [SMILES: CN(C(=O)c1ccn2ncc(-c3cnc(N)nc3)c2c1)c1ccc(C#N)cc1] | 83 |
| 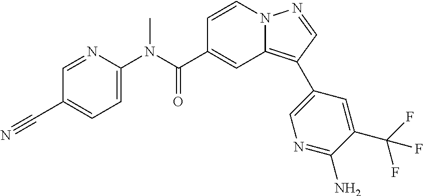 [SMILES: CN(C(=O)c1ccn2ncc(-c3cnc(N)c(C(F)(F)F)c3)c2c1)c1ccc(C#N)cn1] | 84 |
| 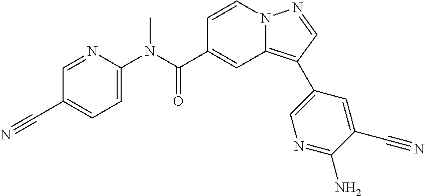 [SMILES: CN(C(=O)c1ccn2ncc(-c3cnc(N)c(C#N)c3)c2c1)c1ccc(C#N)cn1] | 85 |
| 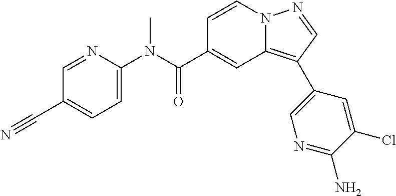 [SMILES: CN(C(=O)c1ccn2ncc(-c3cnc(N)c(Cl)c3)c2c1)c1ccc(C#N)cn1] | 86 |
| 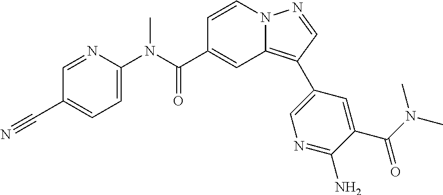 [SMILES: CN(C)C(=O)c1cc(-c2cnn3ccc(C(=O)N(C)c4ccc(C#N)cn4)cc23)cnc1N] | 87 |
| 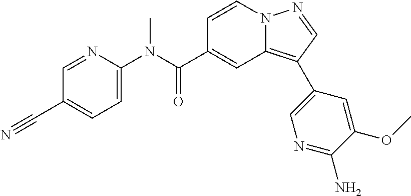 [SMILES: COc1cc(-c2cnn3ccc(C(=O)N(C)c4ccc(C#N)cn4)cc23)cnc1N] | 88 |
| 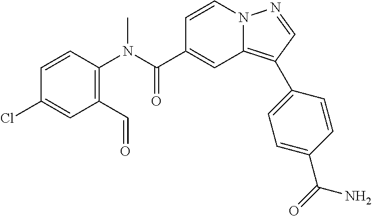 [SMILES: CN(C(=O)c1ccn2ncc(-c3ccc(C(N)=O)cc3)c2c1)c1ccc(Cl)cc1C=O] | 89 |
| 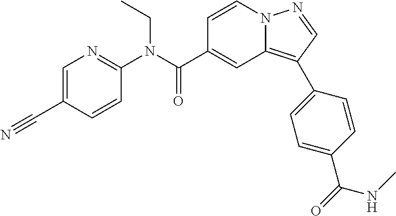 [SMILES: CCN(C(=O)c1ccn2ncc(-c3ccc(C(=O)NC)cc3)c2c1)c1ccc(C#N)cn1] | 90 |
| 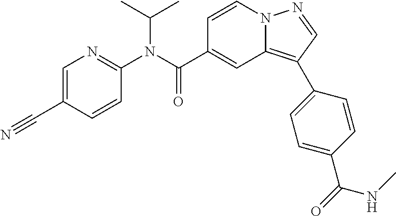 [SMILES: CNC(=O)c1ccc(-c2cnn3ccc(C(=O)N(c4ccc(C#N)cn4)C(C)C)cc23)cc1] | 91 |
| 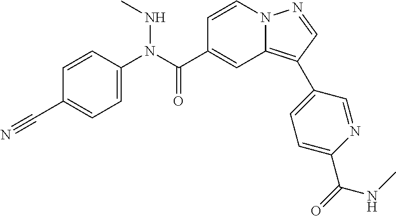 [SMILES: CNC(=O)c1ccc(-c2cnn3ccc(C(=O)N(NC)c4ccc(C#N)cc4)cc23)cn1] | 92 |
| 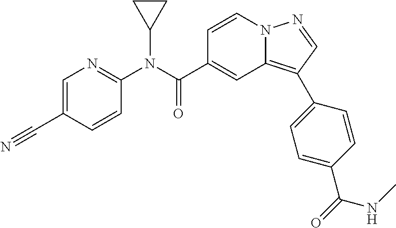 [SMILES: CNC(=O)c1ccc(-c2cnn3ccc(C(=O)N(c4ccc(C#N)cn4)C4CC4)cc23)cc1] | 93 |
| 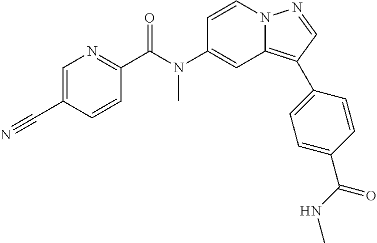 [SMILES: CNC(=O)c1ccc(-c2cnn3ccc(N(C)C(=O)c4ccc(C#N)cn4)cc23)cc1] | 94 |
| 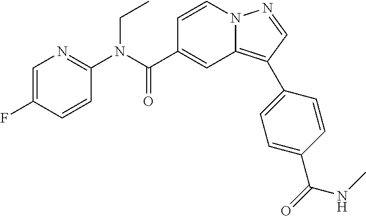 [SMILES: CCN(C(=O)c1ccn2ncc(-c3ccc(C(=O)NC)cc3)c2c1)c1ccc(F)cn1] | 95 |
| 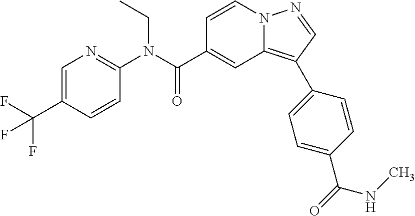 [SMILES: CCN(C(=O)c1ccn2ncc(-c3ccc(C(=O)NC)cc3)c2c1)c1ccc(C(F)(F)F)cn1] | 96 |
| 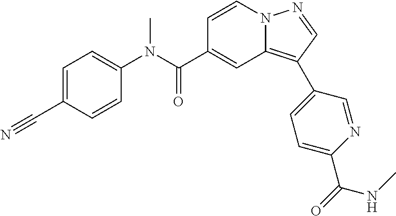 [SMILES: CNC(=O)c1ccc(-c2cnn3ccc(C(=O)N(C)c4ccc(C#N)cc4)cc23)cn1] | 97 |
| 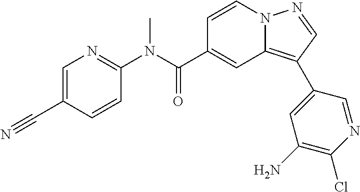 [SMILES: CN(C(=O)c1ccn2ncc(-c3cnc(Cl)c(N)c3)c2c1)c1ccc(C#N)cn1] | 98 |
| 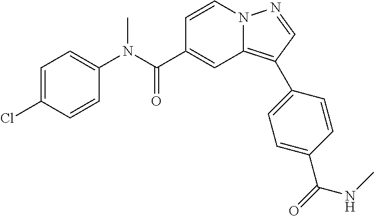 [SMILES: CNC(=O)c1ccc(-c2cnn3ccc(C(=O)N(C)c4ccc(Cl)cc4)cc23)cc1] | 99 |
| 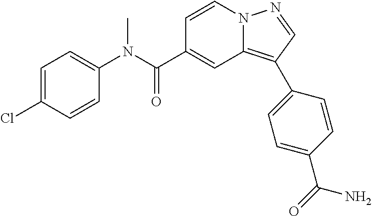 [SMILES: CN(C(=O)c1ccn2ncc(-c3ccc(C(N)=O)cc3)c2c1)c1ccc(Cl)cc1] | 100 |
| 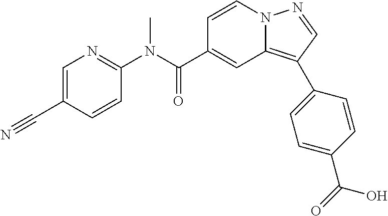 [SMILES: CN(C(=O)c1ccn2ncc(-c3ccc(C(=O)O)cc3)c2c1)c1ccc(C#N)cn1] | 101 |
| 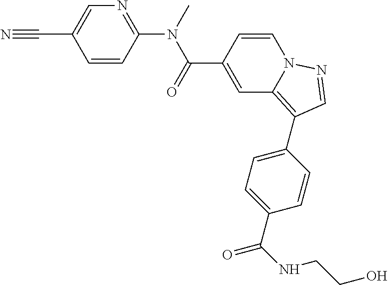 [SMILES: CN(C(=O)c1ccn2ncc(-c3ccc(C(=O)NCCO)cc3)c2c1)c1ccc(C#N)cn1] | 102 |
| 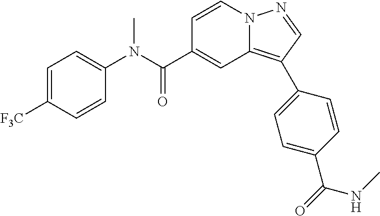 [SMILES: CNC(=O)c1ccc(-c2cnn3ccc(C(=O)N(C)c4ccc(C(F)(F)F)cc4)cc23)cc1] | 103 |
| 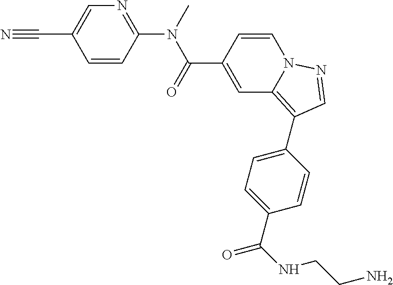 [SMILES: CN(C(=O)c1ccn2ncc(-c3ccc(C(=O)NCCN)cc3)c2c1)c1ccc(C#N)cn1] | 104 |
| 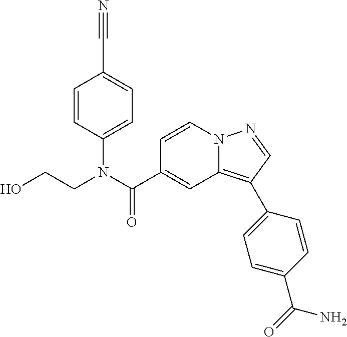 [SMILES: N#Cc1ccc(N(CCO)C(=O)c2ccn3ncc(-c4ccc(C(N)=O)cc4)c3c2)cc1] | 105 |
| 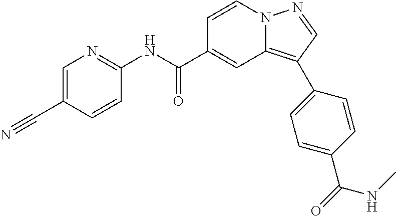 [SMILES: CNC(=O)c1ccc(-c2cnn3ccc(C(=O)Nc4ccc(C#N)cn4)cc23)cc1] | 106 |
| 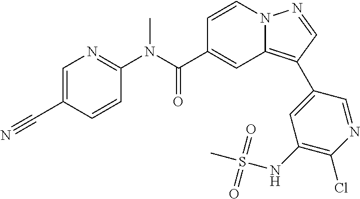 [SMILES: CN(C(=O)c1ccn2ncc(-c3cnc(Cl)c(NS(C)(=O)=O)c3)c2c1)c1ccc(C#N)cn1] | 107 |
| 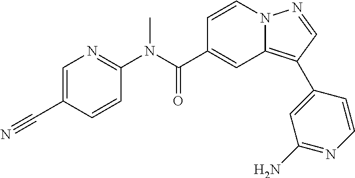 [SMILES: CN(C(=O)c1ccn2ncc(-c3ccnc(N)c3)c2c1)c1ccc(C#N)cn1] | 108 |
| 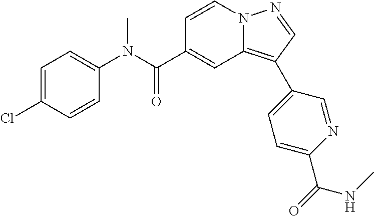 [SMILES: CNC(=O)c1ccc(-c2cnn3ccc(C(=O)N(C)c4ccc(Cl)cc4)cc23)cn1] | 109 |
| 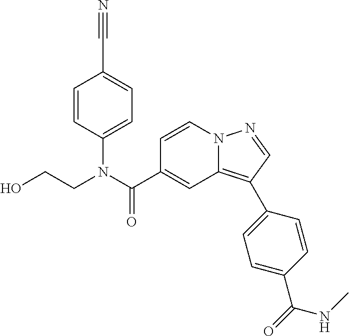 [SMILES: CNC(=O)c1ccc(-c2cnn3ccc(C(=O)N(CCO)c4ccc(C#N)cc4)cc23)cc1] | 110 |
| 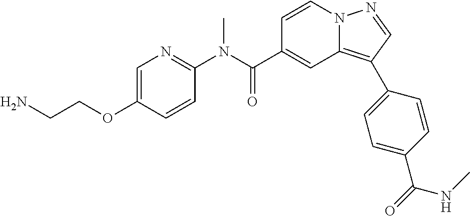 [SMILES: CNC(=O)c1ccc(-c2cnn3ccc(C(=O)N(C)c4ccc(OCCN)cn4)cc23)cc1] | 111 |
| 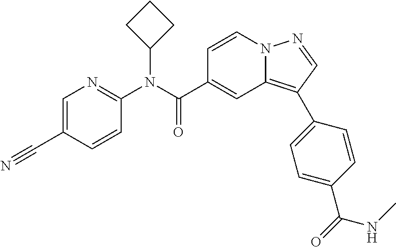 [SMILES: CNC(=O)c1ccc(-c2cnn3ccc(C(=O)N(c4ccc(C#N)cn4)C4CCC4)cc23)cc1] | 112 |
| 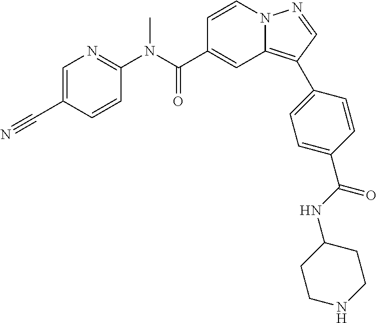 [SMILES: CN(C(=O)c1ccn2ncc(-c3ccc(C(=O)NC4CCNCC4)cc3)c2c1)c1ccc(C#N)cn1] | 113 |
| 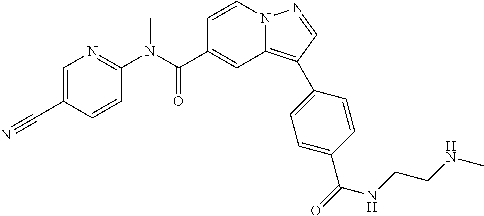 [SMILES: CNCCNC(=O)c1ccc(-c2cnn3ccc(C(=O)N(C)c4ccc(C#N)cn4)cc23)cc1] | 114 |
| 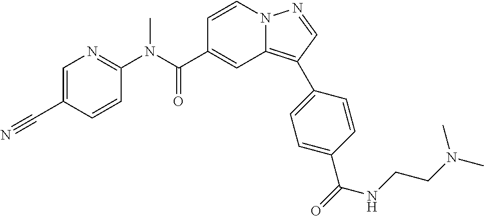 [SMILES: CN(C)CCNC(=O)c1ccc(-c2cnn3ccc(C(=O)N(C)c4ccc(C#N)cn4)cc23)cc1] | 115 |
| 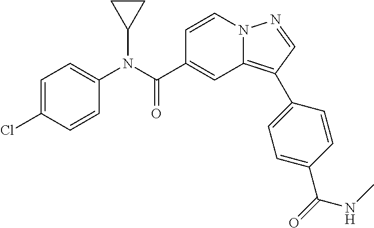 [SMILES: CNC(=O)c1ccc(-c2cnn3ccc(C(=O)N(c4ccc(Cl)cc4)C4CC4)cc23)cc1] | 116 |
| 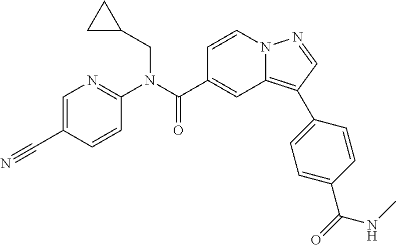 [SMILES: CNC(=O)c1ccc(-c2cnn3ccc(C(=O)N(CC4CC4)c4ccc(C#N)cn4)cc23)cc1] | 117 |
| 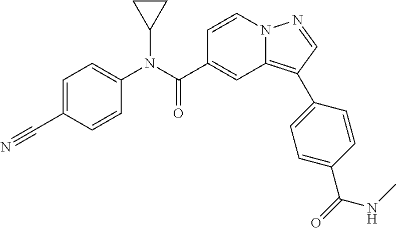 [SMILES: CNC(=O)c1ccc(-c2cnn3ccc(C(=O)N(c4ccc(C#N)cc4)C4CC4)cc23)cc1] | 118 |
| 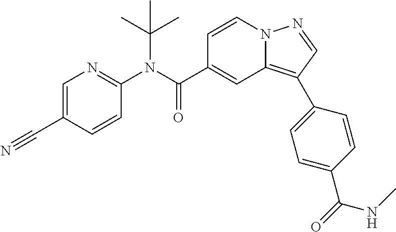 [SMILES: CNC(=O)c1ccc(-c2cnn3ccc(C(=O)N(c4ccc(C#N)cn4)C(C)(C)C)cc23)cc1] | 119 |
| 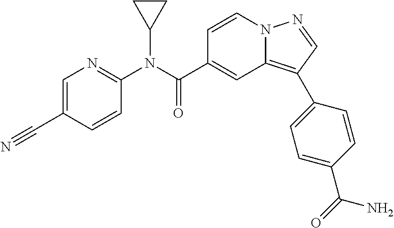 [SMILES: N#Cc1ccc(N(C(=O)c2ccn3ncc(-c4ccc(C(N)=O)cc4)c3c2)C2CC2)nc1] | 120 |
| 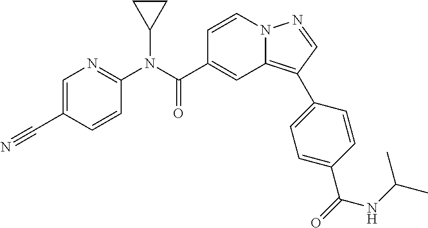 [SMILES: CC(C)NC(=O)c1ccc(-c2cnn3ccc(C(=O)N(c4ccc(C#N)cn4)C4CC4)cc23)cc1] | 121 |
| 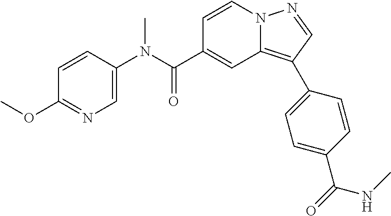 | 122 |
| 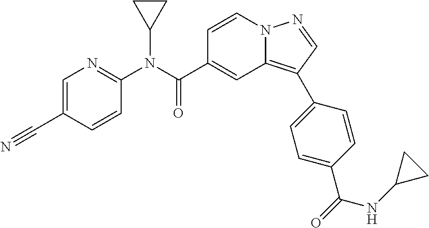 [SMILES: N#Cc1ccc(N(C(=O)c2ccn3ncc(-c4ccc(C(=O)NC5CC5)cc4)c3c2)C2CC2)nc1] | 123 |
| 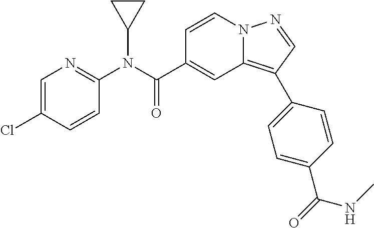 [SMILES: CNC(=O)c1ccc(-c2cnn3ccc(C(=O)N(c4ccc(Cl)cn4)C4CC4)cc23)cc1] | 124 |
| 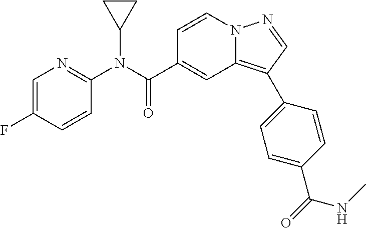 [SMILES: CNC(=O)c1ccc(-c2cnn3ccc(C(=O)N(c4ccc(F)cn4)C4CC4)cc23)cc1] | 125 |
| 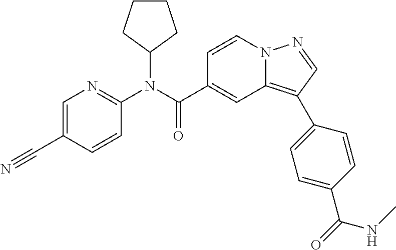 [SMILES: CNC(=O)c1ccc(-c2cnn3ccc(C(=O)N(c4ccc(C#N)cn4)C4CCCC4)cc23)cc1] | 126 |
| 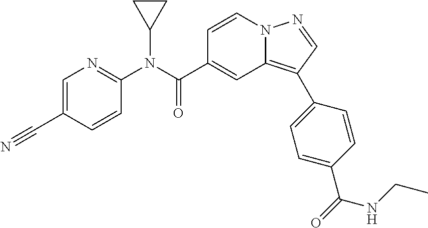 [SMILES: CCNC(=O)c1ccc(-c2cnn3ccc(C(=O)N(c4ccc(C#N)cn4)C4CC4)cc23)cc1] | 127 |
| 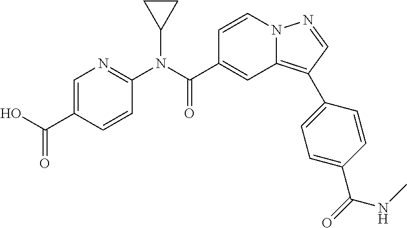 [SMILES: CNC(=O)c1ccc(-c2cnn3ccc(C(=O)N(c4ccc(C(=O)O)cn4)C4CC4)cc23)cc1] | 128 |
| 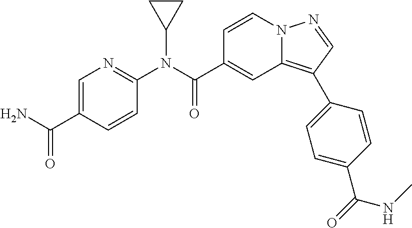 [SMILES: CNC(=O)c1ccc(-c2cnn3ccc(C(=O)N(c4ccc(C(N)=O)cn4)C4CC4)cc23)cc1] | 129 |
| 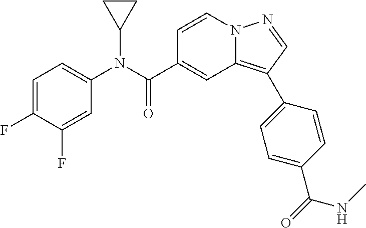 [SMILES: CNC(=O)c1ccc(-c2cnn3ccc(C(=O)N(c4ccc(F)c(F)c4)C4CC4)cc23)cc1] | 130 |
| 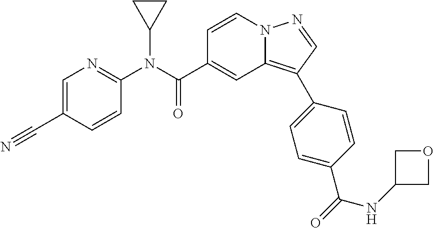 [SMILES: N#Cc1ccc(N(C(=O)c2ccn3ncc(-c4ccc(C(=O)NC5COC5)cc4)c3c2)C2CC2)nc1] | 131 |
| 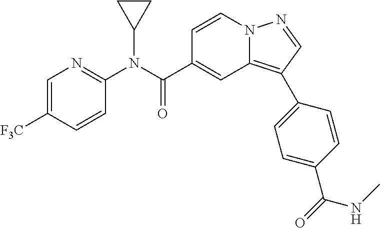 [SMILES: CNC(=O)c1ccc(-c2cnn3ccc(C(=O)N(c4ccc(C(F)(F)F)cn4)C4CC4)cc23)cc1] | 132 |
| 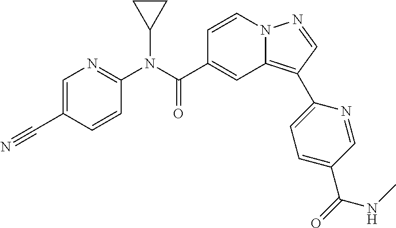 [SMILES: CNC(=O)c1ccc(-c2cnn3ccc(C(=O)N(c4ccc(C#N)cn4)C4CC4)cc23)nc1] | 133 |
| 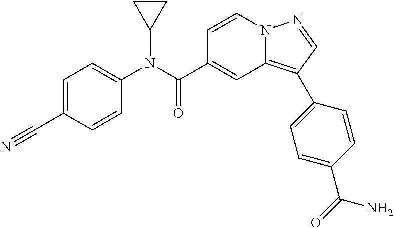 [SMILES: N#Cc1ccc(N(C(=O)c2ccn3ncc(-c4ccc(C(N)=O)cc4)c3c2)C2CC2)cc1] | 134 |
| 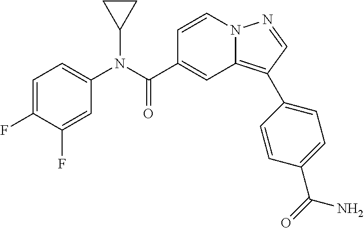 [SMILES: NC(=O)c1ccc(-c2cnn3ccc(C(=O)N(c4ccc(F)c(F)c4)C4CC4)cc23)cc1] | 135 |
| 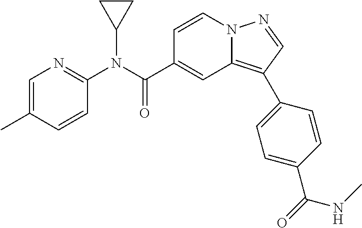 | 136 |
| 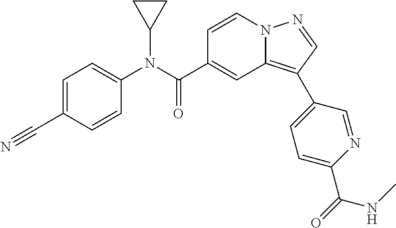 [SMILES: CNC(=O)c1ccc(-c2cnn3ccc(C(=O)N(c4ccc(C#N)cc4)C4CC4)cc23)cn1] | 137 |
| 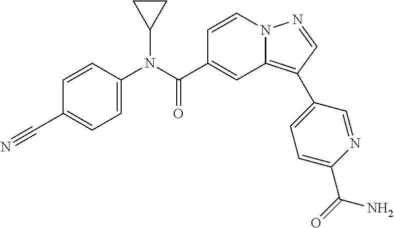 [SMILES: N#Cc1ccc(N(C(=O)c2ccn3ncc(-c4ccc(C(N)=O)nc4)c3c2)C2CC2)cc1] | 138 |
| 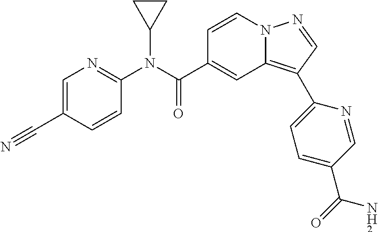 [SMILES: N#Cc1ccc(N(C(=O)c2ccn3ncc(-c4ccc(C(N)=O)cn4)c3c2)C2CC2)nc1] | 139 |
| 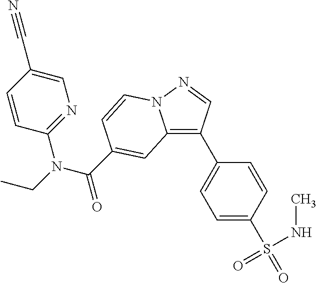 [SMILES: CCN(C(=O)c1ccn2ncc(-c3ccc(S(=O)(=O)NC)cc3)c2c1)c1ccc(C#N)cn1] | 140 |
| 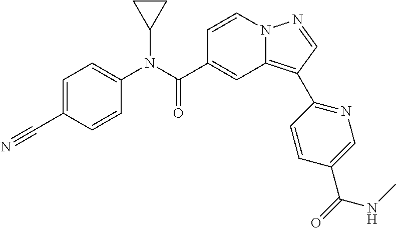 [SMILES: CNC(=O)c1ccc(-c2cnn3ccc(C(=O)N(c4ccc(C#N)cc4)C4CC4)cc23)nc1] | 141 |
| 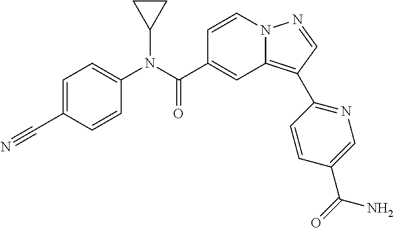 [SMILES: N#Cc1ccc(N(C(=O)c2ccn3ncc(-c4ccc(C(N)=O)cn4)c3c2)C2CC2)cc1] | 142 |
| 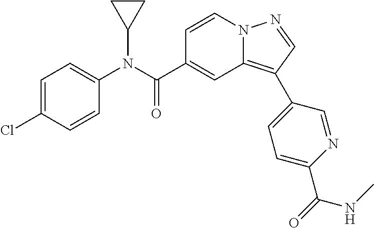 [SMILES: CNC(=O)c1ccc(-c2cnn3ccc(C(=O)N(c4ccc(Cl)cc4)C4CC4)cc23)cn1] | 143 |
| 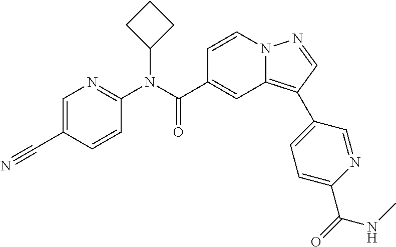 [SMILES: CNC(=O)c1ccc(-c2cnn3ccc(C(=O)N(c4ccc(C#N)cn4)C4CCC4)cc23)cn1] | 144 |
| 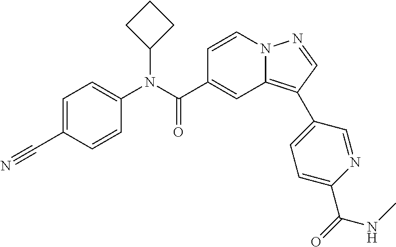 [SMILES: CNC(=O)c1ccc(-c2cnn3ccc(C(=O)N(c4ccc(C#N)cc4)C4CCC4)cc23)cn1] | 145 |
| 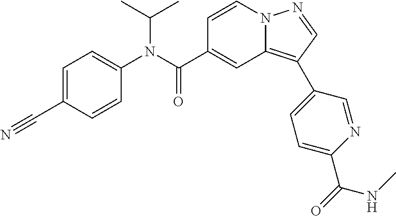 [SMILES: CNC(=O)c1ccc(-c2cnn3ccc(C(=O)N(c4ccc(C#N)cc4)C(C)C)cc23)cn1] | 146 |
| 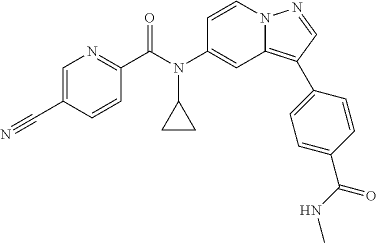 [SMILES: CNC(=O)c1ccc(-c2cnn3ccc(N(C(=O)c4ccc(C#N)cn4)C4CC4)cc23)cc1] | 147 |
| 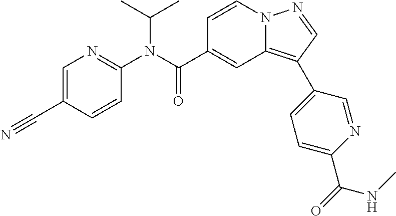 [SMILES: CNC(=O)c1ccc(-c2cnn3ccc(C(=O)N(c4ccc(C#N)cn4)C(C)C)cc23)cn1] | 148 |
| 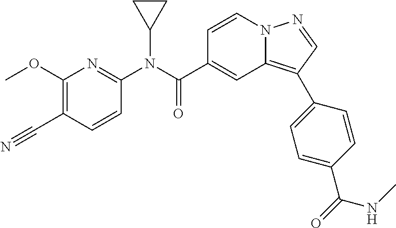 [SMILES: CNC(=O)c1ccc(-c2cnn3ccc(C(=O)N(c4ccc(C#N)c(OC)n4)C4CC4)cc23)cc1] | 149 |
| 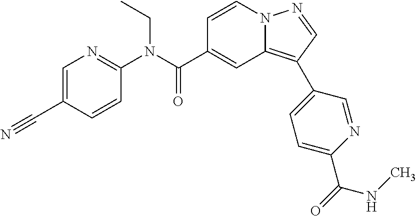 [SMILES: CCN(C(=O)c1ccn2ncc(-c3ccc(C(=O)NC)nc3)c2c1)c1ccc(C#N)cn1] | 150 |
| 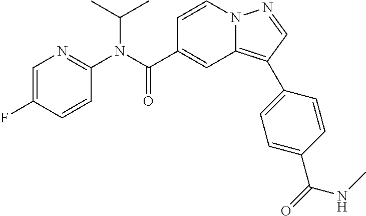 [SMILES: CNC(=O)c1ccc(-c2cnn3ccc(C(=O)N(c4ccc(F)cn4)C(C)C)cc23)cc1] | 151 |
| 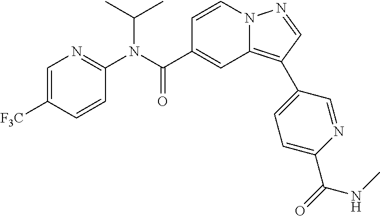 [SMILES: CNC(=O)c1ccc(-c2cnn3ccc(C(=O)N(c4ccc(C(F)(F)F)cn4)C(C)C)cc23)cn1] | 152 |
| 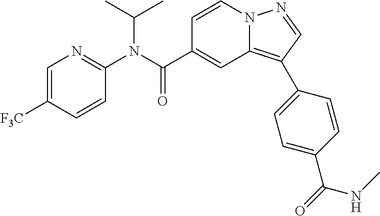 [SMILES: CNC(=O)c1ccc(-c2cnn3ccc(C(=O)N(c4ccc(C(F)(F)F)cn4)C(C)C)cc23)cc1] | 153 |
| 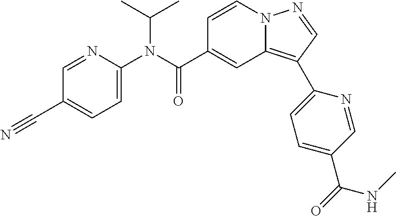 [SMILES: CNC(=O)c1ccc(-c2cnn3ccc(C(=O)N(c4ccc(C#N)cn4)C(C)C)cc23)nc1] | 154 |
| 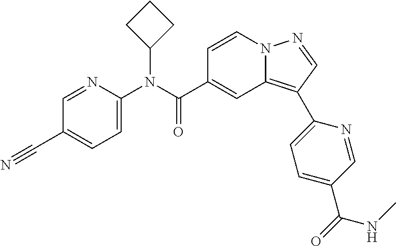 [SMILES: CNC(=O)c1ccc(-c2cnn3ccc(C(=O)N(c4ccc(C#N)cn4)C4CCC4)cc23)nc1] | 155 |
| 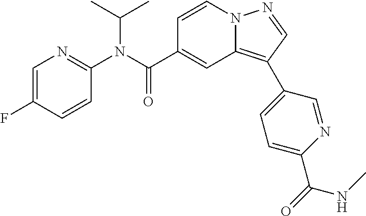 [SMILES: CNC(=O)c1ccc(-c2cnn3ccc(C(=O)N(c4ccc(F)cn4)C(C)C)cc23)cn1] | 156 |
| 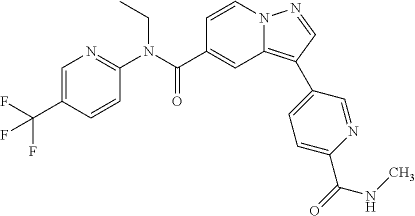 [SMILES: CCN(C(=O)c1ccn2ncc(-c3ccc(C(=O)NC)nc3)c2c1)c1ccc(C(F)(F)F)cn1] | 157 |
| 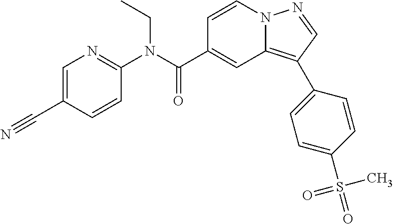 [SMILES: CCN(C(=O)c1ccn2ncc(-c3ccc(S(C)(=O)=O)cc3)c2c1)c1ccc(C#N)cn1] | 158 |
| 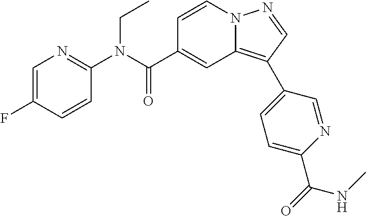 [SMILES: CCN(C(=O)c1ccn2ncc(-c3ccc(C(=O)NC)nc3)c2c1)c1ccc(F)cn1] | 159 |
| 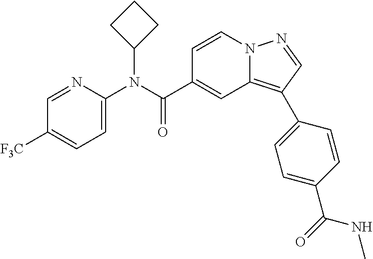 [SMILES: CNC(=O)c1ccc(-c2cnn3ccc(C(=O)N(c4ccc(C(F)(F)F)cn4)C4CCC4)cc23)cc1] | 160 |
| 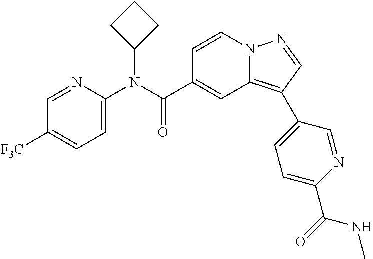 [SMILES: CNC(=O)c1ccc(-c2cnn3ccc(C(=O)N(c4ccc(C(F)(F)F)cn4)C4CCC4)cc23)cn1] | 161 |
| 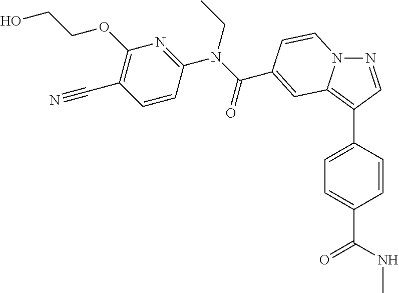 [SMILES: CCN(C(=O)c1ccn2ncc(-c3ccc(C(=O)NC)cc3)c2c1)c1ccc(C#N)c(OCCO)n1] | 162 |
| 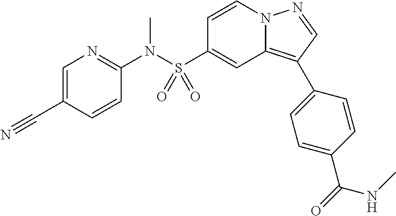 [SMILES: CNC(=O)c1ccc(-c2cnn3ccc(S(=O)(=O)N(C)c4ccc(C#N)cn4)cc23)cc1] | 163 |
| 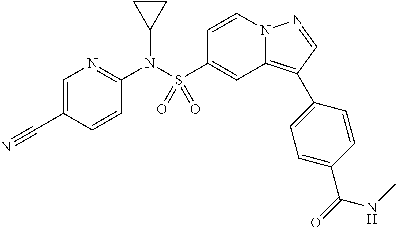 [SMILES: CNC(=O)c1ccc(-c2cnn3ccc(S(=O)(=O)N(c4ccc(C#N)cn4)C4CC4)cc23)cc1] | 164 |
| 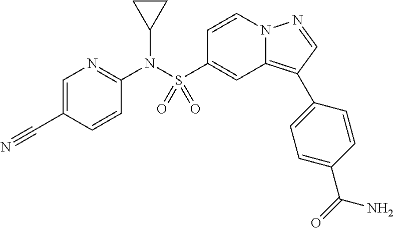 [SMILES: N#Cc1ccc(N(C2CC2)S(=O)(=O)c2ccn3ncc(-c4ccc(C(N)=O)cc4)c3c2)nc1] | 165 |

In another particular embodiment, the compound useful in the method of the invention includes, but is not limited to the following: N-(4-cyanophenyl)-N-methyl-3-(1-methyl-1H-indazol-5-yl)pyrazolo[1,5-a]pyridine-5-carboxamide; (S)-3-(4-(2-aminopropanamido)phenyl)-N-(4-cyanophenyl)-N-methylpyrazolo[1,5-a]pyridine-5-carboxamide; 3-(4-carbamoylphenyl)-N-(5-cyanopyridin-2-yl)-N-methylpyrazolo[1,5-a]pyridine-5-carboxamide; N-(4-cyanophenyl)-N-methyl-3-(1H-pyrrolo[2,3-b]pyridin-5-yl)pyrazolo[1,5-a]pyridine-5-carboxamide; 4-(5-(1-(7-fluoro-3-oxo-2H-benzo[b][1,4]oxazin-4(3H)-yl)ethyl)pyrazolo[1,5-a]pyridin-3-yl)benzamide; N-Methyl-3-(4-(methylcarbamoyl)phenyl)-N-(4-(trifluoromethyl)phenyl)pyrazolo[1,5-a]pyridine-5-carboxamide; N-(4-Chlorophenyl)-N-cyclopropyl-3-(4-(methylcarbamoyl)phenyl)pyrazolo[1,5-a]pyridine-5-carboxamide; N-(5-Cyano-6-methoxypyridin-2-yl)-N-cyclopropyl-3-(4-(methylcarbamoyl)phenyl)pyrazolo [1,5-a]pyridine-5-carboxamide; N-Isopropyl-3-(4-(methylcarbamoyl)phenyl)-N-(5-(trifluoromethyl)pyridin-2-yl)pyrazolo[1,5-a]pyridine-5-carboxamide; and N-cyclobutyl-3-(4-(methylcarbamoyl)phenyl)-N-(5-(trifluoromethyl)pyridin-2-yl)pyrazolo[1,5-a]pyridine-5-carboxamide; or a pharmaceutical acceptable salt or stereoisomer, thereof.

It is noted that the compounds useful in the method of the present invention may be in the form of a pharmaceutically acceptable salt. It is further note that the compounds useful in the method present invention may be a mixture of stereoisomers, or the compound may comprise a single stereoisomer.

In another aspect, the method of the present invention is directed to use of a pharmaceutical composition which includes as an active ingredient a compound according to any one of the above embodiments and variations in combination with a pharmaceutically acceptable carrier, diluent or excipient.

In another embodiment, the pharmaceutical composition is a solid formulation adapted for oral administration. In another embodiment, the composition is a liquid formulation adapted for oral administration. In yet another embodiment, the composition is a tablet. In still another embodiment, the composition is a liquid formulation adapted for parenteral administration.

In yet another embodiment, the pharmaceutical composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In another aspect, the present application is directed to a compound or a pharmaceutical composition according to any one of the above embodiments and variations for use in a therapeutic application.

In another aspect, the present application is directed to a compound or a pharmaceutical composition according to any one of the above embodiments and variations for use as a medicament.

### Enumerated Embodiments

Various enumerated embodiments of the invention are described herein. It will be recognized that features specified in each embodiment may be combined with other specified features to provide further embodiments of the present invention.

In a first embodiment, the invention provides a method for treating, ameliorating, or eradicating the pathology and/or symptomology of cryptosporidiosis caused by a protozoa of the genus Cryptosporidium, comprising administering to a patient in need thereof, a therapeutically effective amount of a compound according to Formula I,

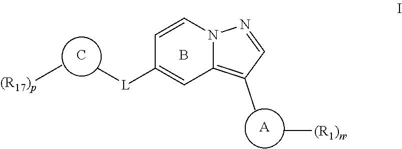

or a pharmaceutical acceptable salt, tautomer or stereoisomer thereof, wherein

n is 0, 1, 2 or 3;

p is 0, 1, 2, or 3;

L is selected from the group consisting of *—(CHR₃)₁₋₃—, *—CHR₃N(R₂)—, CHR₃O—, *—CHR₃S—, *—CHR₃S(O)—, *—CHR₃N(R₂)CHR₃—, *—C(O)—, *—C(O)N(R₂)—, C(O)N(R₂)CHR₃—, *—N(R₂)—, *—N(R₂)CHR₃—, *—N(R₂)C(O)—, *—N(R₂)C(O)N(R₂)—, N(R₂)S(O)₂—, and *—S(O)₂N(R₂)—, wherein
* represents the point of attachment of L to the pyrazolo[1,5-a]pyridine fused ring depicted in Formula I (Ring B);
each R₂ is selected from the group consisting of hydrogen, C₁₋₆alkyl, haloC₁₋₆alkyl, R—C₀₋₄alkylene, and R—C₀₋₄alkylene-C(O)—, wherein R is selected from the group consisting of hydroxyl, C₁₋₄alkoxy, amino, C₁₋₄alkylamino, C₃₋₆cycloalkyl, C₄₋₆heterocycloalkyl, and C₅₋₆heteroaryl, wherein the C₃₋₆cycloalkyl, C₄₋₆heterocycloalkyl, or C₅₋₆heteroaryl of R is unsubstituted or substituted by 1-2 substituents independently selected from the group consisting of halo, amino, hydroxyl, C₁₋₄alkyl, C₁₋₄alkoxy, oxo, and C₅₋₆heteroaryl; and
R₃ is hydrogen or C₁₋₄alkyl;

Ring A is C₆₋₁₀aryl or C₅₋₁₀ heteroaryl;

Ring C is selected from the group consisting of C₆₋₁₀aryl, C₅₋₁₀heteroaryl, C₅₋₇cycloalkyl, C₅₋₇heterocycloalkyl, and a fused bicyclyl comprising a C₅₋₆heterocycloalky fused to a phenyl;

each R₁ is independently selected from the group consisting of halo, cyano, amino, C₁₋₄alkyl, C₁₋₄alkoxy, halo-C₁₋₄alkyl, —C(O)NR₇R₈, —NHC(O)R₁₁, phenyl, C₅₋₆heteroaryl, —C(O)R₁₁, —NHS(O)₂R₁₁, —S(O)₂R₁₁, and —S(O)₂NHR₈, wherein
the phenyl or C₅₋₆heteroaryl of R₁ is unsubstituted or substituted by 1-2 substituents independently selected from the group consisting of C₁₋₄alkyl, amino, halo, and C₁₋₄alkylamino;
R₇ is selected from the group consisting of hydrogen, C₁₋₄alkyl, and haloC₁₋₄alkyl;
R₈ is selected from the group consisting of hydrogen; haloC₁₋₄alkyl; C₃₋₆cycloalkyl; C₄₋₆heterocycloalkyl; C₁₋₄alkyl unsubstituted or substituted by hydroxy, amino, or C₁₋₄alkylamino; and
R₁₁ is selected from the group consisting of hydroxyl and C₁₋₆alkyl unsubstituted or substituted by 1-2 substituents independently selected from the group consisting of amino, C₃₋₆cycloalkyl, and C₄₋₆heterocycloalkyl;

each R₁₇ is selected from the group consisting of cyano, halo, C₁₋₄alkyl, halo-C₁₋₄alkyl, oxo, C₃₋₆cycloalkyl, —S(O)₂C₁₋₄alkyl; C₁₋₄alkoxy unsubstituted or substituted by hydroxy or amino; and —C(O)R₁₂, wherein R₁₂ is hydrogen, hydroxy or amino.

### Embodiment 2

The method according to embodiment 1, wherein

n is 0, 1, 2 or 3;

p is 1 or 2;

L is selected from the group consisting of *—(CHR₃)₁₋₂—, *—CHR₃N(R₂)—, CHR₃O—, *—CHR₃S—, *—CHR₃S(O)—, *—C(O)—, *—C(O)N(R₂)—, *—N(R₂)CHR₃—, *—N(R₂)C(O)—, *—N(R₂)C(O)N(R₂)—, *—N(R₂)S(O)₂—, and *—S(O)₂N(R₂)—, wherein
* represents the point of attachment of L to Ring B;
each R₂ is hydrogen, C₁₋₆alkyl or R—C₀₋₄alkylene, wherein R is selected from the group consisting of hydroxyl, C₁₋₄alkoxy, C₁₋₄alkylamino, C₃₋₆cycloalkyl, C₄₋₆heterocycloalkyl, and C₅₋₆heteroaryl, and
R₃ is hydrogen or C₁₋₄alkyl;

Ring A is C₆₋₁₀aryl or C₅₋₁₀heteroaryl;

Ring C is selected from the group consisting of C₆₋₁₀aryl, C₅₋₁₀heteroaryl, C₅₋₇cycloalkyl, and fused bicyclyl comprising a C₅₋₆heterocycloalky fused to a phenyl;

each R₁ is independently selected from the group consisting of halo, cyano, amino, C₁₋₄alkyl, C₁₋₄alkoxy, halo-C₁₋₄alkyl, —C(O)NR₇R₈, —NHC(O)R₁, C₅₋₆heteroaryl; —C(O)R₁₁, —NHS(O)₂R₁₁, —S(O)₂R₁₁, and —S(O)₂NHR₈, wherein
the C₅₋₆heteroaryl of R, is unsubstituted or substituted by C₁₋₄alkylamino;
R₇ is hydrogen or C₁₋₄alkyl;
R₈ is selected from hydrogen; hydroxy; C₃₋₆cycloalkyl; C₄₋₆heterocycloalkyl; C₁₋₄alkyl unsubstituted or substituted by hydroxy, amino or C₁₋₄alkylamino; and
R₁₁ is hydroxy or C₁₋₆alkyl unsubstituted or substituted by 1-2 substituents independently selected from amino and C₃₋₆cycloalkyl; and

each R₁₇ is independently selected from cyano; halo; C₁₋₄alkyl; halo-C₁₋₄alkyl; oxo; C₃₋₆cycloalkyl; —S(O)₂C₁₋₄alkyl; C₁₋₄alkoxy unsubstituted or substituted by either hydroxyl or amino; and —C(O)R₁₂ wherein R₁₂ is hydrogen, hydroxy or amino.

### Embodiment 3

The method according to Embodiment 1 or Embodiment 2, wherein the compound is capable of inhibiting or modulating the activity of a phosphatidylinositol-4-OH kinase (PI4K) of the cryptosporidium protozoa.

### Embodiment 4

The method according to any one of Embodiments 1 to 4, wherein the cryptosporidium protozoa is Cryptosporidium hominis or Cryptosporidium parvum.

### Embodiment 5

The method according to any one of claims 1 to 4, wherein L is selected from the group consisting of *—(CHR₃)—, *—CHR₃N(R₂)—, *—C(O)—, C(O)N(R₂)—, *—N(R₂)C(O)—, and *—S(O)₂N(R₂)—, wherein

* represents the point of attachment of L to Ring B;

R₂ is hydrogen, C₁₋₆alkyl or R—C₀₋₄alkylene, wherein R is selected from the group consisting of C₁₋₄alkylamino, C₃₋₆cycloalkyl, C₄₋₆heterocycloalkyl, and C₅₋₆heteroaryl; and

R₃ is C₁₋₄alkyl.

### Embodiment 6

The method according to any one of Embodiments 1 to 5, wherein Ring A is selected from the group consisting of phenyl, pyridinyl, pyrimidinyl, pyrrolopyridinyl, and indazolyl.

### Embodiment 7

The method according to any one of claims 1 to 6, wherein Ring C is selected from the group consisting of phenyl, pyridinyl, cyclohexyl, and dihydrobenzooxazinyl.

### Embodiment 8

The method according to any one of claims 1 to 7, wherein each R₁ is independently selected from the group consisting of halo, cyano, amino, C₁₋₄alkyl, C₁₋₄alkoxy, halo-C₁₋₄alkyl, —C(O)NR₇R₈, and —NHC(O)R₁₁, wherein

R₇ and R₈ are independently hydrogen or C₁₋₄alkyl;

R₁₁ is C₁₋₆alkyl unsubstituted or substituted by 1-2 substituents independently selected from the group consisting of amino and C₃₋₆cycloalkyl.

### Embodiment 9

The method according to any one of embodiments 1 to 8, wherein each R₁₇ is independently selected from the group consisting of cyano, halo, C₁₋₄alkyl, halo-C₁₋₄alkyl, oxo, C₁₋₄alkoxy, and —C(O)H.

### Embodiment 10

The method of claim 1, wherein the compound is of Formula Ia:

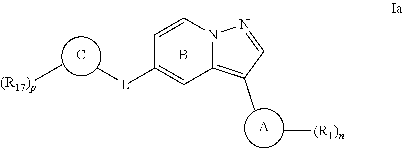

or a pharmaceutical acceptable salt, tautomer or stereoisomer thereof, wherein

n is 0 or 1;

p is 1 or 2;

L is *—CHR₃— or *—C(O)NR₂—; wherein
* represents the point of attachment of L to Ring B;
R₂ is C₁₋₄alkyl or C₃₋₆cycloalkyl; and
R₃ is C₁₋₄alkyl;

Ring A is phenyl or C₅₋₁₀heteroaryl;

Ring C is phenyl, C₅₋₁₀heteroaryl or fused bicyclyl comprising a C₅₋₆heterocycloalky fused to a phenyl;

each R₁ is independently C₁₋₄alkyl, —NHC(O)R₁₁, or —C(O)NR₇R₈, wherein
R₇ and R₈ is independently hydrogen or C₁₋₄alkyl;
R₁₁ is C₁₋₄alkyl substituted by —NH₂; and

each R₁₇ is independently selected from the group consisting of halo, cyano, C₁₋₄alkyl, haloC₁₋₄alkyl, and C₁₋₄alkoxy.

### Embodiment 11

The method of claim 10, wherein

L is *—CHCH₃—, *—C(O)N(CH₃)—, *—C(O)NCH(CH₃)₂—, *—C(O)N(cyclopropyl)-, or *—C(O)N(cyclobutyl)-;

Ring A is selected from the group consisting of phenyl, pyridinyl, pyrrolopyridinyl, and indazolyl,

Ring C is phenyl, pyridinyl or dihydrobenzooxazinyl;

each R₁ is independently selected from the group consisting of methyl, —C(O)NH₂, —C(O)NHCH₃, or —NHC(O)CH(NH₂)CH₃; and

each R₁₇ is independently selected from the group consisting of cyano, fluoro, chloro, methyl, trifluoromethyl, methoxy, and oxo.

### Embodiment 12

The method according to claim 1, wherein the compound is selected from the group of compounds listed in Table I.

### Embodiment 13

A method for treating, inhibiting, ameliorating, or eradicating the pathology and/or symptomology of cryptosporidiosis caused by a cryptosporidium protozoa, comprising administering to a patient in need thereof a therapeutically effective amount of an agent capable of modulating or inhibiting the activity of a phosphatidylinositol-4-OH kinase (PI4K) of said protozoa.

### Embodiment 14

The method of claim 13, wherein the crypotosporidium protozoa is Cryptosporidium hominis or Cryptosporidium parvum.

### Embodiment 15

The method of claim 13 or 14, wherein the agent is a compound is a compound according to any one of claims 1 to 12.

As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²p, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by Plasdmodium or (ii) associated with Plasdmodium activity, or (iii) characterized by activity (normal or abnormal) of Plasdmodium or (2) reduce or inhibit the activity of Plasdmodium; or (3) reduce or inhibit the growth of Plasdmodium. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of Plasdmodium; or at least partially reducing or inhibiting the growth of Plasdmodium.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

In general, compounds useful for the method of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Biological Assays

The activity of a compound used in the method of the present invention for inhibition of parasitemai in host cells can be assessed by the following assays. It is understood that the assays illustrate the invention without in any way limiting the scope of the invention.

Culturing and Maintaining Host Cells and Cryptosporidium Parasite

Human ileocecal colorectal adenocarcinoma cells (HCT-8 [HRT-18] ATCC, CCL-34) were maintained in T-175 flasks (Corning, 431080) in complete growth medium (RPMI-1640 medium (Gibco, 11875) supplemented with 10% heat-inactivated horse serum (Gibco, 26050), 1×MEM non-essential amino acids (Gibco, 11140), 10 mM HEPES (Gibco, 15630), 100 units/mL penicillin, and 100 units/mL streptomycin) at 37° C. and 5% CO₂ in a humidified incubator. Cultures were passaged twice weekly using 10 mL of 1×Phosphate-Buffered Saline (PBS) without Ca²⁺ and Mg²⁺ (Gibco, 20012) for washing and 3-5 mL per T-175 flask of TrypLE Express Enzyme (Gibco, 12604) for dissociation of adherent cells.

Cryptosporidium parvum oocysts purchased from the Sterling Laboratory, University of Arizona (Iowa isolate) were purified from infected calf feces using discontinuous sucrose and cesium chloride centrifugation gradients and stored in PBS solution containing 0.01% Tween 20, 100 units/mL penicillin and 100 units/mL gentamicin.

Cryptosporidium hominis oocysts were purchased from the Tufts University Cummings School of Veterinary Medicine (courtesy of Dr. Saul Tzipori). C. hominis oocysts were purified from infected gnotobiotic piglet feces and stored in PBS solution containing 0.01% Tween-20, 100 units/mL penicillin and 100 units/mL gentamicin. C. parvum and C. hominis oocysts less than three months old from the date of shedding were used in infection experiments.

Excystation and Infection:

Excystation and infection protocols were developed following established methods with some modifications (Gut & Nelson, 1999, Upton et aL, 1995, Bessoff et aL, 2013). Briefly, oocysts were primed in 1 mL of 10 mM hydrochloric acid in 1× Hank's Balanced Salt Solution (HBSS) (Gibco, 14025) for 10 minutes with agitation at 1000 rpm, 37° C. on an Eppendorf thermomixer, then washed twice with 1 mL of room temperature non-acidic 1× HBSS by centrifugation at 13,000 rpm for 3 minutes at 25° C. Primed oocysts were further excysted at a concentration of 1×10⁶ oocysts/μL in parasite infection medium consisting of a pre-warmed and pre-gassed 1:1 formulation of Leibovitz's L-15 medium (Gibco, 11415) and UltraCULTURE medium (Lonza, 12-725F) supplemented with 2 mM sodium taurocholate (Sigma, 86339-1), 10% heat-inactivated horse serum, and 200 μM L-ascorbic acid (Sigma, 95210) at 25° C. for 10 minutes. HCT-8 monolayer cells were infected with excysted cryptosporidium at a specified multiplicity of infection (MOI). All dilutions for subsequent assays were performed in parasite infection medium without sodium taurocholate. Pre-excysted oocysts were enumerated microscopically using a C-Chip disposable hemocytometer (NanoEnTek, DHC-N01).

Compound and Assay Plate Preparation:

Compound powders were dissolved in neat DMSO (Fisher, D4121) to 10 mM and stored at 4° C. prior to dilution into source plates. Dilutions were carried out using a Microlab STAR liquid handler (Hamilton) to obtain compound source plates containing the ten-point or eight-point three fold dilutions starting from 10 mM in duplicates. Source plates were stored at 4° C. prior to spotting into assay plates. Before administration, all compound source plates were equilibrated to room temperature. A specified volume of compounds from source plate were spotted to assay plate using an Echo Acoustic liquid handler (LABCYTE, 550) so that the final DMSO concentration was less than 0.5%. Each assay plate a specified number of DMSO-treated negative control wells and a well-studied potent active compound at 100 nM as positive control. As a quality control, all positive and negative-control wells were used to calculate a Z′-value and signal to noise ratio (S:N) for each plate.

IC₅₀ Determination by Cytopathic Effect (CPE) Based Assay:

Cryptosporidium spp are obligate-intracellular parasites that infect intestinal epithelial cells and the host cell is killed upon parasite egress. In patients, cryptosporidium infection has been shown to induce severe villous atrophy caused by the loss of villous enterocytes. The loss of epithelial cells is due to both rapid parasite invasion/multiplication/egress and also pro-inflammatory immune response (Adams et al., 1994, Griffiths et al., 1994). We have observed a consistent cytopathic effect (CPE) in HCT-8 cells with C. spp infection the loss of viability of the host cells using CellTiter-Glo reagent.

Confluent HCT-8 cells in T-175 flasks were directly infected with excysted oocysts at an MOI (host to parasite) of 1:2 for C. parvum and 1:4 for C. hominis. The number of host-cells is determined using a NucleoCounter (Chemometec, NC-100) in a control flask. Infected monolayers were incubated for 3 hours at 37° C., followed by gentle washing once with 10 mL of 1×PBS before dissociation with 3-5 mL of TrypLE. Infected cell pellet was re-suspended in 90% complete growth medium and 10% parasite infection medium without sodium taurocholate. 2.5×10⁴ batch-infected HCT-8 cells were seeded in each well of a 384-well plate (Greiner, 789091) in a total well volume of 30 μL using a MultiDrop liquid handler (ThermoScientific, 5840300). All plates were incubated for 24 hours at 37° C. prior to compound administration. Compounds were spotted at various concentrations at 60 nL per well from the source plates using an Echo Acoustic liquid handler (LABCYTE, 550) and treatment allowed to proceed for 48 hours. Following compound treatment, assay plates were allowed to equilibrate to room temperature for one hour in a biosafety cabinet to minimize temperature gradient effects. Cells were lysed and host cell viability measured by addition of 20 μL per well of Cell-Titer Glo 2.0 (Promega, G9243) using the Multidrop. The luminescence reading was measured at the rate of 0.1 seconds per well by a Clarity Luminometer (BioTek). Raw data files were exported and results were expressed as percent stimulation where 100% stimulation was equal to the mean of the active control wells and 0% stimulation was equal to the mean of the DMSO-treated negative control wells. Cell viability curves were analyzed using Novartis software.

The effectiveness of selected compounds to minimize the cytopathic effect of both Cryptosporidium hominis and Cryptosporidium parvum were measured. The result were reported in Table II, C. parvum in the first column [(Cp CPE EC₅₀ (μM)] and C. hominis in the fourth column. [(Ch CPE EC₅₀ (μM)]. The effectiveness ranges from no effect to nanomolar concentration.

IC₅₀ Determination by High Content Imaging (HCl) Assay:

Infection and Compound Treatment:

Imaging assays were developed following established Cryptosporidium spp labeling and in vitro infection models with some modifications (Bessoff et aL, 2013, Gut & Nelson, 1999). Briefly, 2×10⁴ HCT-8 cells per well were seeded into 384-well, flat black clear-bottom OPERA assay plates (Greiner, 789071-G) at 20 μL per well in complete growth medium using a Multidrop Combi liquid handler (ThermoScientific, 5840300) and standard tube dispensing cassette (ThermoScientific, 24072670) and incubated for 24 hours at 37° C. The HCT-8 cells were infected with 10 μL per well of 1×10⁴ excysted C. parvum oocysts (host to parasite MOI of 1:0.5) or 10 μL per well of 4×10⁴ excysted C. hominis oocysts (MOI 1:2) in parasite infection medium using the Multidrop and incubated at 37° C. 24 hours post-infection, 60 nL of compounds were spotted in each well using an Echo Acoustic liquid handler (LABCYTE, 550) as described above and the plates were incubated for 48 hours at 37° C.

Fixation and Labeling:

Following compound treatment, cells were washed twice with PBS, fixed with 40 μL of 4% paraformaldehyde (Electron Microscopy Sciences, 15710) in PBS for 20 minutes at 25° C. and washed with PBS followed by PBS-FT (PBS containing 1% fetal bovine serum in PBS and 0.05% Tween-20. To ensure monolayers are uncompromised, all aspiration steps were performed allowing for a 15 μL remaining well volume. The fixed cells were permeabilized and blocked PBS-FT for 30 minutes at 25° C. For staining 4 μg/mL Streptavidin-conjugated Alexa Fluor 568 (Life Technologies, S11226) was mixed with 2 μg/mL biotinylated Vicia villosa lectin (Vector Laboratories, B-1235) in PBS-FT and incubated at 25° C. for 1 hour. The bound label was filtered through a pre-equilibrated syringe filter (Sartorius Stedim, 16534-K). To label the intracellular parasitic life stages the permeabilized cells were incubated with 20 μL Alexa568-VVL for 1 hour at 25° C. The labelled cells were washed with PBS-FT followed by a PBS wash. Finally HCT-8 host cell nuclei were counterstained with 5 μM Draq-5 (Abcam, ab108410) diluted in PBS and stored before detection.

Detection:

Once labeled, the plates were imaged using an Opera QEHS (PerkinElmer™). Imaging was performed at 10× using a Nikon UPlan Apo lens. Nine images were collected in each well covering more than 80% of the well surface. The samples were exposed to 561 nm and 635 nm laser lines to excite respectively the Alexa Fluor@ 598 conjugated lectins and DRAQ5™. The laser power was selected at 2250 μW, exposure time set at 800 milliseconds and focal height set at 5 μm. The fluorescence signal was then collected on cooled CCD cameras after passing the emitted light through a quad-band primary dichroic (405/488/561/635) and a detection dichroic (510) followed by emission filers 600/40 and a 690/50 to collect the light emitted respectively by the labeled parasite and nuclei.

Analysis:

Images were analyzed using a custom analysis script written in Acapella® (PerkinElmer™). In brief, nuclei were detected and the mask obtained was then dilated to encompass the cell cytoplasm. These objects were thereafter referred as cell-bodies. The average signal from the images collected for the parasite channel was measured for each cell body. Cells were then classified as infected vs. not-infected by applying an intensity cut-off and for each well the number of cells and the percentage of infected cells was calculated. The cut-off used to classify the cells as infected vs. not infected was automatically optimized using the positive and negative controls, using an ‘R’ (Team, 2015). In brief, the cut-off was set as the intensity threshold which maximized the Z′ factor (Zhang et aL, 1999). Results were expressed as percent inhibition where 100% inhibition was equal to the mean of the active control wells and 0% inhibition was equal to the mean of the DMSO-treated negative control wells. The data was analyzed with the Novartis in-house software (Helios software application, Novartis Institutes for BioMedical Research, unpublished) using the methods described in the following references (Fomenko et al., 2006, Kelly & Rice, 1990, Normolle, 1993, Sebaugh, 2011) (Kahm et al., 2010). After manual curation to address any potential screening patterns or artifacts, each well data point was normalized using the control wells so that no effect was set to 0% and full inhibition was set to −100%. The data was then curve fitted in Helios software to calculate the active concentration which resulted in having only 50% of the cells infected.

The result of the assay of selected compounds on C. parvum were reported on the Table II, second column [Cp HCl IC₅₀ (μM)] Selected compounds exhibit sub-micro molar activities in preventing infection of the host cells.

Determination of Cytotoxicity

Cytotoxicity against HepG2 (ATCC# HB-8065), a human liver cancer cell line, was determined as previously described earlier (Manjunatha et al., 2015). Briefly, cells were seeded at a density of 10⁵ cells per well, incubated at 37° C. for 24 h and exposed to two-fold serially-diluted compounds for 5 days. Cell viability was monitored using the Cell Proliferation Kit II (Invitrogen).

The cytotoxicity values of selected compounds are reported in the fifth column [HepG2 CC₅₀ (μM)] of Table II. The results show the compounds are generally safe.

PI(4)K Enzymatic Assay

Baculovirus Expression and Purification of C. parvum Phosphatidylinositol 4-Kinase:

The full-length coding sequence of C. parvum PI(4)K (cgd8_4500, 1114 amino acids) was codon-optimized for baculovirus expression, synthesized and cloned into pFastBac-HTb (Invitrogen 10584-027) in frame with the amino-terminal polyhistidine tag using the BamHI and Hindlll restriction sites. Recombinant pFastBacHTb-CpPI(4)K bacmid clones were generated by site-specific transposition in E. coli DH10Bac (Invitrogen 10361-012). The bacmid sequence was confirmed by direct DNA sequencing to confirm a lack of mutations across the whole gene. The subsequent steps for bacmid isolation, transfection and selection of the recombinant viruses were performed according to the manufacturer's protocol (Bac-to-Bac system #10359, Invitrogen).

SF9 cells, cultured in SF-900 III serum-free medium, were transfected with recombinant baculovirus at 1/200 (v/v) and incubated at 27° C. for 72 h. The pellets were collected after centrifugation and re-suspended in cell lysis buffer (20 mM Tris-HCl, pH 7.5, 300 mM NaCl, 1 mM DTT, 20 mM imidazole, 0.01% Triton X-100 and 1× complete protease inhibitor cocktail without EDTA (Roche Diagnostics 04693116001)). The cell suspension was lysed by sonication and the clarified supernatant was loaded onto a 1 ml HisTrap affinity column (GE Healthcare) pre-equilibrated with buffer A (20 mM Tris-HCl, pH 7.5, 300 mM NaCl, 1 mM DTT, 20 mM Imidazole, and 1× complete protease inhibitor cocktail without EDTA). The column was washed with buffer B (buffer A containing 45 mM imidazole) and the bound protein of interest was eluted with buffer C (buffer A with 90 mM imidazole). The fractions containing CpPI(4)K were pooled, concentrated using Amicon Ultra-15 and purified by a gel-filtration column (Hi-Load 26/60 Superdex 200, GE Healthcare) equilibrated with 20 mM Tris, pH 7.5, 300 mM NaCl, 1 mM DTT and 1× protease inhibitor cocktail without EDTA. The concentrations of the purified protein (Mw 132.39 kda) was determined by using the protein molar extinction coefficient (ε_{280 nm}=133,810 M⁻¹ cm⁻¹). Aliquots were flash frozen in liquid nitrogen and immediately stored at −80° C.

PI(4)K Enzymatic Assay:

The CpPI(4)K enzymatic assay was performed as described earlier with a some modifications (McNamara et al., 2013). Briefly, L-α-phosphatidylinositol (Avanti Polar Lipid 840046), dissolved in 3% n-octylglucoside (Roche Diagnostics 10634425001), was used as the lipid substrate for the PI(4)K activity assay. CpPI(4)K was assayed using Transcreener ADP₂ FP detection kit (BellBrook 3010) in a black, solid 384-well plate (Corning 3575). The final assay volume was 10 μl and contained 3 nM of the respective CpPI(4)K construct in 10 mM Tris, pH 7.5, 1 mM DTT, 3 μM ATP, 5 mM Mn2+, 0.05% Triton X-100 and 10 μM phosphatidylinositol/octylglucoside. The enzyme reaction was performed for 50 minutes at room temperature and was stopped by adding 10 μl of detection mix containing 1× stop buffer (50 mM HEPES, pH7.5, 400 mM NaCl, 20 mM EDTA, and 0.02% Brij-35), 2 nM AMP Alexa Fluor 633 tracer, and 20 μg ml⁻¹ ADP antibody. Fluorescence polarization measurements were performed on the Infinite M1000 plate reader (Tecan) with λex=635 nm and λem=680 nm (20-nm bandwidth). IC₅₀ values were calculated using Graphpad Prism software.

The inhibitory concentration (IC₅₀) of selected compounds on the C. parvPI(4)K activity is provided in the third column [Cp_PI4K_enz IC50 (μM)] of Table II. These compounds exhibit sub-micro molar inhibitory values and are hence potent inhibitors of C. parvum PI(4)K enzyme.

TABLE II
Results of the Biological Assays

| | | | | | HepG2 |
|---|---|---|---|---|---|
| Example | Cp CPE | Cp HCl | Cp_PI4K_enz | Ch CPE | CC₅₀ |
| No.* | EC₅₀ (μM) | IC₅₀ (μM) | IC₅₀ (μM) | EC₅₀ (μM) | (μM) |
| 1 | 0.660 | | | | |
| 2 | >20.000 | | | | |
| 3 | 16.490 | | | | |
| 4 | 6.784 | | | | |
| 5 | >20.000 | | | | |
| 6 | | | | | |
| 7 | >20.000 | | | | |
| 8 | | | | | |
| 9 | | | | | |
| 10 | | | | | |
| 11 | | | | | |
| 12 | 12.632 | | | | |
| 13 | 1.543 | | | | >50.000 |
| 14 | | | | | |
| 15 | >20.000 | | | | |
| 16 | >20.000 | | | | |
| 17 | 0.058 | | | | 2.509 |
| 18 | | | | | |
| 19 | 0.037 | | 0.003 | | 5.908 |
| 20 | 0.105 | | | | 5.418 |
| 21 | >20.000 | | | | |
| 22 | >20.000 | | | | |
| 23 | | | | | |
| 24 | >20.000 | | | | |
| 25 | 2.303 | | | | |
| 26 | 0.142 | | | | |
| 27 | 0.815 | | 0.140 | | |
| 28 | 2.818 | | | | 9.905 |
| 29 | >20.000 | | | | |
| 30 | >20.000 | | | | |
| 31 | >20.000 | | | | |
| 32 | 0.093 | 0.058 | 0.025 | 0.119 | 15.841 |
| 33 | 1.513 | | | | |
| 34 | 4.872 | | | | |
| 35 | >20.000 | | | | |
| 36 | >20.000 | | | | |
| 37 | 3.788 | | | | |
| 38 | 4.709 | | | | |
| 39 | >20.000 | | | | |
| 40 | 0.074 | | | | |
| 41 | 0.097 | | | | 24.131 |
| 42 | 0.142 | | | | |
| 43 | | | | | |
| 44 | | | | | |
| 45 | 0.332 | | 0.239 | | |
| 46 | | | | | |
| 47 | >20.000 | | | | |
| 48 | 6.301 | | | | |
| 49 | 19.154 | | | | |
| 50 | >20.000 | | | | |
| 51 | >20.000 | | | | |
| 52 | | | | | |
| 53 | 0.550 | | 0.047 | | |
| 54 | 0.324 | | 0.074 | | >50.000 |
| 55 | 7.297 | | | | |
| 56 | 0.692 | | | | |
| 57 | 5.588 | | | | |
| 58 | | | | | |
| 59 | >20.000 | | | | |
| 60 | 2.366 | | | | |
| 61 | 17.845 | | | | |
| 62 | 0.028 | | | | |
| 63 | 0.195 | | | | |
| 64 | 0.143 | | | | 4.840 |
| 65 | 5.121 | | | | 2.281 |
| 66 | 0.132 | | | | |
| 67 | 0.026 | | | | |
| 68 | 19.578 | | | | |
| 69 | 0.201 | | | | |
| 71 | 0.571 | | | | 1.817 |
| 72 | 1.396 | | | | 5.419 |
| 73 | 0.964 | | | | |
| 74 | 1.237 | | | | |
| 75 | 0.066 | | 0.003 | | 19.690 |
| 76 | >20.000 | | | | |
| 77 | 0.582 | | 0.050 | | 29.312 |
| 79 | 0.205 | | | | |
| 80 | 1.174 | | | | |
| 81 | 0.683 | | | | |
| 82 | >20.000 | | | | >50.000 |
| 83 | 0.487 | | | | >50.000 |
| 84 | 4.270 | | | | |
| 85 | 0.639 | | | | 30.358 |
| 86 | 0.501 | | | | |
| 87 | 0.415 | | | | >50.000 |
| 88 | 2.246 | | | | |
| 89 | 0.085 | | | | |
| 90 | 0.068 | 0.052 | 0.008 | | 14.822 |
| 91 | 0.129 | | 0.021 | 0.139 | 18.706 |
| 92 | 0.310 | 0.094 | 0.057 | | 8.515 |
| 93 | 0.152 | | | | 28.077 |
| 94 | 0.368 | 0.123 | 0.117 | 0.456 | 31.231 |
| 95 | 0.630 | | | | 12.954 |
| 96 | 0.050 | 0.024 | 0.003 | | 7.139 |
| 97 | 0.241 | | 0.073 | | 31.114 |
| 98 | 2.211 | | | | 10.198 |
| 99 | 0.018 | | | | 6.509 |
| 100 | 0.021 | | | | 13.309 |
| 101 | 15.151 | | | | >50.000 |
| 102 | 0.399 | | | | 38.469 |
| 103 | 0.007 | 0.008 | | | 8.058 |
| 104 | 3.614 | | | | >50.000 |
| 105 | >20.000 | | | | >50.000 |
| 106 | >20.000 | | | | >50.000 |
| 107 | | | | | 39.732 |
| 108 | 4.938 | | | | 14.199 |
| 109 | 0.129 | | | | 16.851 |
| 110 | >20.000 | | | | 23.772 |
| 111 | >20.000 | | | | >50.000 |
| 112 | 0.270 | | 0.072 | 0.351 | 5.015 |
| 113 | 19.404 | | | | 2.669 |
| 114 | 9.126 | | | | 5.524 |
| 115 | 3.016 | | | | 2.703 |
| 116 | 0.007 | | | | 3.006 |
| 117 | 0.505 | | 0.007 | | 4.331 |
| 118 | 0.026 | | | | 5.899 |
| 119 | 1.930 | | 0.488 | 1.937 | 20.074 |
| 120 | 0.172 | | 0.040 | 0.276 | 23.733 |
| 121 | 8.641 | | 0.860 | >20.000 | 33.236 |
| 122 | >20.000 | | | | 15.825 |
| 123 | 2.994 | | 0.038 | 3.154 | 17.241 |
| 124 | 0.103 | 0.060 | 0.014 | | 21.013 |
| 125 | 0.709 | | | | 25.453 |
| 126 | 0.410 | | 0.071 | | 22.067 |
| 127 | 0.102 | 0.062 | 0.003 | 0.071 | 40.217 |
| 128 | >20.000 | | | | >50.000 |
| 129 | >20.000 | | | | >50.000 |
| 130 | 0.235 | | | | >50.000 |
| 131 | 2.732 | | 0.329 | 4.110 | >50.000 |
| 132 | 0.089 | | | | 17.258 |
| 133 | 2.178 | | | | 20.872 |
| 134 | 0.042 | | | | 7.242 |
| 135 | 0.279 | | | | 31.114 |
| 136 | 0.430 | | | | 6.320 |
| 137 | 0.350 | | | | 2.191 |
| 138 | 0.490 | | | | 8.101 |
| 139 | 2.074 | | | | 16.681 |
| 140 | 10.338 | | | | 26.061 |
| 141 | 0.344 | | | | 20.810 |
| 142 | 0.379 | | | | 18.349 |
| 143 | 0.079 | | | | 17.512 |
| 144 | 1.788 | | | | 7.689 |
| 145 | 0.417 | | | | 16.836 |
| 146 | 0.329 | | | | 32.108 |
| 147 | >20.000 | | | | 32.746 |
| 148 | 1.865 | | | | 20.065 |
| 149 | 0.055 | 0.032 | 0.004 | | 23.273 |
| 150 | 1.068 | 0.382 | 0.219 | | 9.820 |
| 151 | 2.312 | | | | 29.181 |
| 152 | 0.456 | | | | 14.941 |
| 153 | 0.039 | 0.011 | 0.006 | 0.070 | 20.623 |
| 154 | 0.964 | | | | 45.735 |
| 155 | 1.012 | | | | >50.000 |
| 156 | 15.553 | | | | >50.000 |
| 157 | 0.295 | | | | 8.956 |
| 158 | 3.753 | | | | 23.010 |
| 159 | 5.365 | | | | 9.815 |
| 160 | 0.085 | 0.029 | 0.008 | | 2.091 |
| 161 | 0.775 | | | | 1.219 |
| 162 | | | | | 24.383 |
| 163 | 0.197 | | | | 8.483 |
| 164 | | | | | 6.977 |
| 165 | 0.607 | | | | 6.374 |

*Same Example no. as is in WO 2014/078802

### Preparation of the Compounds of the Invention

The process for preparing the compounds listed in Table 1 is descrbied in detailed on pages 66 to 258 of WO 2014/078802 A1. Also included in the publication are the physical properties of the compounds

1. A method for treating, ameliorating, or eradicating the pathology and/or symptomology of cryptosporidiosis caused by a protozoa of the genus Cryptosporidium, comprising administering to a patient in need thereof, a therapeutically effective amount of a compound according to Formula I, 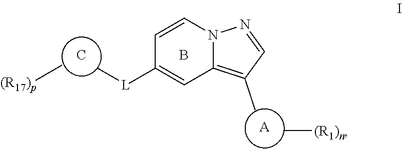
or a pharmaceutical acceptable salt, tautomer or stereoisomer thereof, wherein
n is 0, 1,2 or 3;
p is 0, 1, 2, or 3;
L is selected from the group consisting of *—(CHR₃)₁₋₃—, *—CHR₃N(R₂)—, *—CHR₃O—, *—CHR₃S—, *—CHR₃S(O)—, *—CHR₃N(R)CHR₃—, *—C(O)—, *—C(O)N(R₂)—, *—C(O)N(R₂)CHR₃—, *—N(R₂)—, *—N(R₂)CHR₃—, *—N(R₂)C(O)—, *—N(R₂)C(O)N(R₂)—, *—N(R₂)S(O)₂—, and *—S(O)₂N(R₂)—, wherein
* represents the point of attachment of L to the pyrazolo[1,5-a]pyridine fused ring depicted in Formula I (Ring B);
each R₂ is selected from the group consisting of hydrogen, C₁₋₆alkyl, haloC₁₋₆alkyl, R—C₀₋₄alkylene, and R—C₀₋₄alkylene-C(O)—, wherein R is selected from the group consisting of hydroxyl, C₁₋₄alkoxy, amino, C₁₋₄alkylamino, C₃₋₆cycloalkyl, C₄₋₆heterocycloalkyl, and C₅₋₆heteroaryl, wherein the C₃₋₆cycloalkyl, C₄₋₆heterocycloalkyl, or C₅₋₆heteroaryl of R is unsubstituted or substituted by 1-2 substituents independently selected from the group consisting of halo, amino, hydroxyl, C₁₋₄alkyl, C₁₋₄alkoxy, oxo, and C₅₋₆heteroaryl; and
R₃ is hydrogen or C₁₋₄alkyl;
Ring A is C₆₋₁₀aryl or C₅₋₁₀heteroaryl;
Ring C is selected from the group consisting of C₆₋₁₀aryl, C₅₋₁₀heteroaryl, C₅₋₇cycloalkyl, C₅₋₇heterocycloalkyl, and a fused bicyclyl comprising a C₅₋₆heterocycloalky fused to a phenyl;
each R₁ is independently selected from the group consisting of halo, cyano, amino, C₁₋₄alkyl, C₁₋₄alkoxy, halo-C₁₋₄alkyl, —C(O)NR₇R₈, —NHC(O)R₁₁, phenyl, C₅₋₆heteroaryl, —C(O)R₁₁, —NHS(O)₂R₁₁, —S(O)₂R₁₁, and —S(O)₂NHR₈, wherein
the phenyl or C₅₋₆heteroaryl of R₁ is unsubstituted or substituted by 1-2 substituents independently selected from the group consisting of C₁₋₄alkyl, amino, halo, and C₁₋₄alkylamino;
R₇ is selected from the group consisting of hydrogen, C₁₋₄alkyl, and haloC₁₋₄alkyl;
R₈ is selected from the group consisting of hydrogen; haloC₁₋₄alkyl; C₃₋₆cycloalkyl; C₄₋₆heterocycloalkyl; C₁₋₄alkyl unsubstituted or substituted by hydroxy, amino, or C₁₋₄alkylamino; and
R₁₁ is selected from the group consisting of hydroxyl and C₁₋₆alkyl unsubstituted or substituted by 1-2 substituents independently selected from the group consisting of amino, C₃₋₆cycloalkyl, and C₄₋₆heterocycloalkyl;
each R₁₇ is selected from the group consisting of cyano, halo, C₁₋₄alkyl, halo-C₁₋₄alkyl, oxo, C₃₋₆cycloalkyl, —S(O)₂C₁₋₄alkyl; C₁₋₄alkoxy unsubstituted or substituted by hydroxy or amino; and —C(O)R₁₂, wherein R₁₂ is hydrogen, hydroxy or amino.

2. The method according to claim 1, wherein
n is 0, 1,2 or 3;
p is 1 or 2;
L is selected from the group consisting of *—(CHR₃)₁₋₂—, *—CHR₃N(R₂)—, *—CHR₃O—, *—CHR₃S—, *—CHR₃S(O)—, *—C(O)—, *—C(O)N(R₂)—, *—N(R₂)CHR₃—, *—N(R₂)C(O)—, *—N(R₂)C(O)N(R₂)—, *—N(R₂)S(O)₂—, and *—S(O)₂N(R₂)—, wherein
* represents the point of attachment of L to Ring B;
each R₂ is hydrogen, C₁₋₆alkyl or R—C₀₋₄alkylene, wherein R is selected from the group consisting of hydroxyl, C₁₋₄alkoxy, C₁₋₄alkylamino, C₃₋₆cycloalkyl, C₄₋₆heterocycloalkyl, and C₅₋₆heteroaryl, and
R₃ is hydrogen or C₁₋₄alkyl;
Ring A is C₆₋₁₀aryl or C₅₋₁₀heteroaryl;
Ring C is selected from the group consisting of C₆₋₁₀aryl, C₅₋₁₀heteroaryl, C₅₋₇cycloalkyl, and fused bicyclyl comprising a C₅₋₆heterocycloalky fused to a phenyl;
each R₁ is independently selected from the group consisting of halo, cyano, amino, C₁₋₄alkyl, C₁₋₄alkoxy, halo-C₁₋₄alkyl, —C(O)NR₇R₈, —NHC(O)R₁₁, C₅₋₆heteroaryl; —C(O)R₁₁, —NHS(O)₂R₁₁, —S(O)₂R₁₁, and —S(O)₂NHR₈, wherein
the C₅₋₆heteroaryl of R₁ is unsubstituted or substituted by C₁₋₄alkylamino;
R₇ is hydrogen or C₁₋₄alkyl;
R₈ is selected from hydrogen; hydroxy; C₃₋₆cycloalkyl; C₄₋₆heterocycloalkyl; C₁₋₄alkyl unsubstituted or substituted by hydroxy, amino or C₁₋₄alkylamino; and
R₁₁ is hydroxy or C₁₋₆alkyl unsubstituted or substituted by 1-2 substituents independently selected from amino and C₃₋₆cycloalkyl; and
each R₁₇ is independently selected from cyano; halo; C₁₋₄alkyl; halo-C₁₋₄alkyl;
oxo; C₃₋₆cycloalkyl; —S(O)₂C₁₋₄alkyl; C₁₋₄alkoxy unsubstituted or substituted by either hydroxyl or amino; and —C(O)R₁₂ wherein R₁₂ is hydrogen, hydroxy or amino.

3. The method according to claim 1, wherein the compound is capable of inhibiting or modulating the activity of a phosphatidylinositol-4-OH kinase (PI4K) of the cryptosporidium protozoa.

4. The method according to claim 1, wherein the cryptosporidium protozoa is Cryptosporidium hominis or Cryptosporidium parvum.

5. The method according to claim 1, wherein L is selected from the group consisting of *—(CHR₃)—, *—CHR₃N(R₂)—, *—C(O)—, *—C(O)N(R₂)—, *—N(R₂)C(O)—, and *—S(O)₂N(R₂)—, wherein
* represents the point of attachment of L to Ring B;
R₂ is hydrogen, C₁₋₆alkyl or R—C₀₋₄alkylene, wherein R is selected from the group consisting of C₁₋₄alkylamino, C₃₋₆cycloalkyl, C₄₋₆heterocycloalkyl, and C₅₋₆heteroaryl; and
R₃ is C₁₋₄alkyl.

6. The method according to claim 1, wherein Ring A is selected from the group consisting of phenyl, pyridinyl, pyrimidinyl, pyrrolopyridinyl, and indazolyl.

7. The method according to claim 1, wherein Ring C is selected from the group consisting of phenyl, pyridinyl, cyclohexyl, and dihy drobenzooxazinyl.

8. The method according to claim 1, wherein each R₁ is independently selected from the group consisting of halo, cyano, amino, C₁₋₄alkyl, C₁₋₄alkoxy, halo-C₁₋₄alkyl, —C(O)NR₇R₈, and —NHC(O)R₁₁, wherein
R₇ and R₈ are independently hydrogen or C₁₋₄alkyl;
R₁₁ is C₁₋₆alkyl unsubstituted or substituted by 1-2 substituents independently selected from the group consisting of amino and C₃₋₆cycloalkyl.

9. The method according to claim 1, wherein each R₁₇ is independently selected from the group consisting of cyano, halo, C₁₋₄alkyl, halo-C₁₋₄alkyl, oxo, C₁₋₄alkoxy, and —C(O)H.

10. The method according to claim 1, wherein the compound is of Formula Ia: 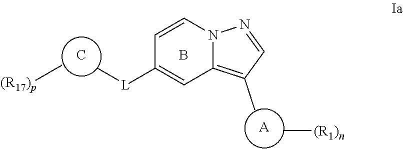
or a pharmaceutical acceptable salt, tautomer or stereoisomer thereof,
wherein
n is 0 or 1;
p is 1 or 2;
L is *—CHR₃— or *—C(O)NR₂—; wherein
* represents the point of attachment of L to Ring B;
R₂ is C₁₋₄alkyl or C₃₋₆cycloalkyl; and
R₃ is C₁₋₄alkyl;
Ring A is phenyl or C₅₋₁₀heteroaryl;
Ring C is phenyl, C₅₋₁₀heteroaryl or fused bicyclyl comprising a C₅₋₆heterocycloalky fused to a phenyl;
each R₁ is independently C₁₋₄alkyl, —NHC(O)R₁₁, or —C(O)NR₇R₈, wherein
R₇ and R₈ is independently hydrogen or C₁₋₄alkyl;
R₁₁ is C₁₋₄alkyl substituted by —NH₂; and
each R₁₇ is independently selected from the group consisting of halo, cyano, C₁₋₄alkyl, haloC₁₋₄alkyl, and C₁₋₄alkoxy.

11. The method according to claim 10, wherein
L is *—CHCH₃—, *—C(O)N(CH₃)—, *—C(O)NCH(CH₃)₂—, *—C(O)N(cyclopropyl)-, or *—C(O)N(cyclobutyl)-;
Ring A is selected from the group consisting of phenyl, pyridinyl, pyrrolopyridinyl, and indazolyl;
Ring C is phenyl, pyridinyl or dihydrobenzooxazinyl;
each R₁ is independently selected from the group consisting of methyl, —C(O)NH₂, —C(O)NHCH₃, or —NHC(O)CH(NH₂)CH₃; and
each R₁₇ is independently selected from the group consisting of cyano, fluoro, chloro, methyl, trifluoromethyl, methoxy, and oxo.

12. The method according to claim 1, wherein the compound is selected from the group consisting of:
N-(4-cyanophenyl)-N-methyl-3-(4-(trifluoromethyl)phenyl)pyrazolo[1,5-a]pyridine-5-carboxamide;
4-fluoro-N-methyl-N-((3-(4-(trifluoromethyl)phenyl)pyrazolo[1,5-a]yridine-5-yl)methyl)aniline;
N-(4-chlorophenyl)-N-methyl-3-(4-(trifluoromethyl)phenyl)pyrazolo[1,5-a]pyridine-5-carboxamide;
N-(4-fluorophenyl)-N-methyl-3-(4-(trifluoromethyl)phenyl)pyrazolo[1,5-a]pyridine-5-carboxamide;
N-methyl-N-(5-methylpyridin-2-yl)-3-(4-(trifluoromethyl)phenyl)pyrazolo[1,5-a]pyridine-5-carboxamide;
4-chloro-N-methyl-N-((3-(4-(trifluoromethyl)phenyl)pyrazolo[1,5-a]yridine-5-yl)methyl)aniline;
N,5-dimethyl-N-((3-(4-(trifluoromethyl)phenyl)pyrazolo[1,5-a]yridine-5-yl)methyl)yridine-2-amine;
5-((4-fluorophenoxy)methyl)-3-(4-(trifluoromethyl)phenyl)pyrazolo[1,5-a]pyridine;
N-(4-cyanophenyl)-N-(2-methoxyethyl)-3-(4-(trifluoromethyl)phenyl)pyrazolo[1,5-a]pyridine-5-carboxamide;
N-(4-cyanophenyl)-N-(2-(dimethylamino)ethyl)-3-(4-(trifluoromethyl)phenyl)pyrazolo[1,5-a]pyridine-5-carboxamide;
N-(4-cyanophenyl)-N-((tetrahydro-2H-pyran-4-yl)methyl)-3-(4-(trifluoromethyl)phenyl)pyrazolo[1,5-a]pyridine-5-carboxamide;
N-(4-(methylsulfonyl)phenyl)-N-((tetrahydro-2H-pyran-4-yl)methyl)-3-(4-(trifluoromethyl)phenyl)pyrazolo[1,5-a]pyridine-5-carboxamide;
N-(5-cyanopyridin-2-yl)-N-methyl-3-(4-(trifluoromethyl)phenyl)pyrazolo[1,5-a]pyridine-5-carboxamide;
N-methyl-N-(5-methylpyridin-3-yl)-3-(4-(trifluoromethyl)phenyl)pyrazolo[1,5-a]pyridine-5-carboxamide;
5-(((5-methylpyridin-2-yl)oxy)methyl)-3-(4-(trifluoromethyl)phenyl)pyrazolo[1,5-a]pyridine;
5-(4-fluorophenethyl)-3-(4-(trifluoromethyl)phenyl)pyrazolo[1,5-a]pyridine;
N-(4-cyanophenyl)-N-methyl-3-(1-methyl-1H-indazol-5-yl)pyrazolo[1,5-a]pyridine-5-carboxamide;
3-(6-acetamidopyridin-3-yl)-N-(4-cyanophenyl)-N-methylpyrazolo[1,5-a]pyridine-5-carboxamide;
3-(4-carbamoylphenyl)-N-(4-cyanophenyl)-N-methylpyrazolo[1,5-a]pyridine-5-carboxamide;
3-(4-carbamoylphenyl)-N-(4-fluorophenyl)-N-methylpyrazolo[1,5-a]pyridine-5-carboxamide
5-(((4-fluorophenyl)thio)methyl)-3-(4-(trifluoromethyl)phenyl)pyrazolo[1,5-a]pyridine;
5-(((4-fluorophenyl)sulfinyl)methyl)-3-(4-(trifluoromethyl)phenyl)pyrazolo[1,5-a]pyridine;
3-(4-(1H-pyrazol-5-yl)phenyl)-N-(4-cyanophenyl)-N-methylpyrazolo[1,5-a]pyridine-5-carboxamide;
N-(4-cyanophenyl)-N-methyl-3-(5-(trifluoromethyl)pyridine-2-yl)pyrazolo[1,5-a]pyridine-5-carboxamide;
N-(5-cyanopyridin-2-yl)-N-methyl-3-(5-(trifluoromethyl)pyridine-2-yl)pyrazolo[1,5-a]pyridine-5-carboxamide;
(S)-3-(4-(2-aminopropanamido)phenyl)-N-(4-cyanophenyl)-N-methylpyrazolo[1,5-a]pyridine-5-carboxamide;
3-(5-carbamoylpyridin-2-yl)-N-(4-cyanophenyl)-N-methylpyrazolo[1,5-a]pyridine-5-carboxamide;
4-cyano-N-methyl-N-(3-(4-(trifluoromethyl)phenyl)pyrazolo[1,5-a]yridine-5-yl)benzamide;
4-fluoro-N-methyl-N-(3-(4-(trifluoromethyl)phenyl)pyrazolo[1,5-a]yridine-5-yl)benzamide;
4-cyano-N-methyl-N-(3-(4-(trifluoromethyl)phenyl)pyrazolo[1,5-a]pyridine-5-yl)benzenesulfonamide;
4-fluoro-N-methyl-N-(3-(4-(trifluoromethyl)phenyl)pyrazolo[1,5-a]pyridine-5-yl)benzenesulfonamide;
3-(4-carbamoylphenyl)-N-(5-cyanopyridin-2-yl)-N-methylpyrazolo[1,5-a]pyridine-5-carboxamide;
N-methyl-3-(4-(trifluoromethyl)phenyl)-N-(5-(trifluoromethyl)pyridine-2-yl)pyrazolo[1,5-a]pyridine-5-carboxamide;
N-methyl-N-(5-(methylsulfonyl)pyridine-2-yl)-3-(4-(trifluoromethyl)phenyl)pyrazolo[1,5-a]pyridine-5-carboxamide;
N-(4-fluorobenzyl)-3-(4-(trifluoromethyl)phenyl)pyrazolo[1,5-a]pyridine-5-amine;
N-(4-fluorobenzyl)-N-methyl-3-(4-(trifluoromethyl)phenyl)pyrazolo[1,5-a]pyridine-5-amine;
N-methyl-6-(trifluoromethyl)-N-(3-(4-(trifluoromethyl)phenyl)pyrazolo[1,5-a]yridine-5-yl)nicotinamide;
N-methyl-5-(trifluoromethyl)-N-(3-(4-(trifluoromethyl)phenyl)pyrazolo[1,5-a]yridine-5-yl)picolinamide;
4-cyano-N-((tetrahydro-2H-pyran-4-yl)methyl)-N-(3-(4-(trifluoromethyl)phenyl)pyrazolo[1,5-a]pyridin-5-yl)benzamide;
N-(4-cyanophenyl)-N-methyl-3-(1H-pyrrolo[2,3-b]pyridin-5-yl)pyrazolo[1,5-a]pyridine-5-carboxamide;
3-(6-aminopyridin-3-yl)-N-(4-cyanophenyl)-N-methylpyrazolo[1,5-a]pyridine-5-carboxamide;
3-(4-aminophenyl)-N-(4-cyanophenyl)-N-methylpyrazolo[1,5-a]pyridine-5-carboxamide;
3-(4-(2-aminoacetamido)phenyl)-N-(4-cyanophenyl)-N-methylpyrazolo[1,5-a]pyridine-5-carboxamide;
(R)-3-(4-(2-aminopropanamido)phenyl)-N-(4-cyanophenyl)-N-methylpyrazolo[1,5-a]pyridine-5-carboxamide;
(S)-3-(4-(2-amino-3-methylbutanamido)phenyl)-N-(4-cyanophenyl)-N-methylpyrazolo[1,5-a]pyridine-5-carboxamide;
(S)-3-(4-(2-amino-2-cyclohexylacetamido)phenyl)-N-(4-cyanophenyl)-N-methylpyrazolo[1,5-a]pyridine-5-carboxamide;
3-(4-fluorophenyl)-1-methyl-1-(3-(4-(trifluoromethyl)phenyl)pyrazolo[1,5-a]pyridin-5-yl)urea;
6-(1,1-difluoroethyl)-N-methyl-N-(3-(4-(trifluoromethyl)phenyl)pyrazolo[1,5-a]pyridin-5-yl)nicotinamide;
6-cyclopropyl-N-methyl-N-(3-(4-(trifluoromethyl)phenyl)pyrazolo[1,5-a]pyridin-5-yl)nicotinamide;
4-cyclopropyl-N-methyl-N-(3-(4-(trifluoromethyl)phenyl)pyrazolo[1,5-a]pyridin-5-yl)benzamide;
5-fluoro-N-methyl-N-(3-(4-(trifluoromethyl)phenyl)pyrazolo[1,5-a]pyridin-5-yl)picolinamide;
N-methyl-4-(methylsulfonyl)-N-(3-(4-(trifluoromethyl)phenyl)pyrazolo[1,5-a]pyridin-5-yl)benzamide;
N-(5-cyanopyridin-2-yl)-N-methyl-3-(1H-pyrrolo[2,3-b]pyridin-5-yl)pyrazolo[1,5-a]pyridine-5-carboxamide;
3-(6-aminopyridin-3-yl)-N-(5-cyanopyridin-2-yl)-N-methylpyrazolo[1,5-a]pyridine-5-carboxamide;
4-chloro-N-methyl-N-(3-(4-(trifluoromethyl)phenyl)pyrazolo[1,5-a]pyridin-5-yl)benzamide;
N-(3-(4-carbamoylphenyl)pyrazolo[1,5-a]pyridin-5-yl)-4-fluoro-N-methylbenzamide;
4-fluoro-N-methyl-N-(3-(4-(5-(methylamino)-1,3,4-thiadiazol-2-yl)phenyl)pyrazolo[1,5-a]pyridin-5-yl)benzamide;
N-methyl-N-(5-(methylsulfonyl)pyridin-2-yl)-3-(1H-pyrrolo[2,3-b]pyridin-5-yl)pyrazolo[1,5-a]pyridine-5-carboxamide;
N-(5-cyanopyridin-2-yl)-N-methyl-3-(5-methylpyridin-2-yl)pyrazolo[1,5-a]pyridine-5-carboxamide;
N-(5-cyanopyridin-2-yl)-3-(5-methoxypyridin-2-yl)-N-methylpyrazolo[1,5-a]pyridine-5-carboxamide;
3-(5-carbamoylpyridin-2-yl)-N-(5-cyanopyridin-2-yl)-N-methylpyrazolo[1,5-a]pyridine-5-carboxamide;
3-(4-carbamoylphenyl)-N-methyl-N-(5-(trifluoromethyl)pyridin-2-yl)pyrazolo[1,5-a]pyridine-5-carboxamide;
3-(4-carbamoylphenyl)-N-methyl-N-(5-methylpyridin-2-yl)pyrazolo[1,5-a]pyridine-5-carboxamide;
N-(4-fluorophenyl)-N-methyl-3-(4-(methylcarbamoyl)phenyl)pyrazolo[1,5-a]pyridine-5-carboxamide;
4-(5-(1-(methyl(5-methylpyridin-2-yl)amino)ethyl)pyrazolo[1,5-a]pyridin-3-yl)benzamide;
4-(5-(1-(7-fluoro-3-oxo-2H-benzo[b][1,4]oxazin-4(3H)-yl)ethyl)pyrazolo[1,5-a]pyridin-3-yl)benzamide;
N-(4-cyanophenyl)-N-methyl-3-(4-(methylcarbamoyl)phenyl)pyrazolo[1,5-a]pyridine-5-carboxamide;
N-(4-(5-(1-(methyl(5-methylpyridin-2-yl)amino)ethyl)pyrazolo[1,5-a]pyridin-3-yl)phenyl)acetamide;
3-(4-acetamidophenyl)-N-(5-cyanopyridin-2-yl)-N-methylpyrazolo[1,5-a]pyridine-5-carboxamide;
tert-butyl methyl(3-(4-(methylcarbamoyl)phenyl)pyrazolo [1,5-a]pyridin-5-yl)carbamate;
4-(5-(7-fluoro-3,4-dihydro-2H-benzo[b][1,4]oxazine-4-carbonyl)pyrazolo[1,5-a]pyridin-3-yl)benzamide;
4-(5-(7-fluoro-3-methyl-3,4-dihydro-2H-benzo[b][1,4]oxazine-4-carbonyl)pyrazolo[1,5-a]pyridin-3-yl)benzamide;
4-(5-(7-fluoro-3,4-dihydro-2H-benzo[b][1,4]oxazine-4-carbonyl)pyrazolo[1,5-a]pyridin-3-yl)-N-methylbenzamide;
4-(5-(7-fluoro-3-methyl-3,4-dihydro-2H-benzo[b][1,4]oxazine-4-carbonyl)pyrazolo [1,5-a]pyridin-3-yl)-N-methylbenzamide;
N-(5-cyanopyridin-2-yl)-N-methyl-3-(4-(methylcarbamoyl)phenyl)pyrazolo[1,5-a]pyridine-5-carboxamide;
N-(5-cyanopyridin-2-yl)-3-(4-(methylcarbamoyl)phenyl)-N-(oxetan-3-yl)pyrazolo[1,5-a]pyridine-5-carboxamide;
N-(1-(1H-pyrazol-1-yl)propan-2-yl)-3-(4-carbamoylphenyl)-N-(5-cyanopyridin-2-yl)pyrazolo[1,5-a]pyridine-5-carboxamide;
3-(6-amino-5-fluoropyridin-3-yl)-N-(4-cyanophenyl)-N-methylpyrazolo[1,5-a]pyridine-5-carboxamide;
3-(4-amino-3,5-dimethylphenyl)-N-(4-cyanophenyl)-N-methylpyrazolo[1,5-a]pyridine-5-carboxamide;
3-(6-amino-5-methylpyridin-3-yl)-N-(4-cyanophenyl)-N-methylpyrazolo[1,5-a]pyridine-5-carboxamide;
3-(4-carbamoylphenyl)-N-(4-cyanocyclohexyl)-N-methylpyrazolo[1,5-a]pyridine-5-carboxamide;
3-(2-aminopyrimidin-5-yl)-N-(4-cyanophenyl)-N-methylpyrazolo[1,5-a]pyridine-5-carboxamide;
3-(6-amino-5-(trifluoromethyl)pyridin-3-yl)-N-(5-cyanopyridin-2-yl)-N-methylpyrazolo[1,5-a]pyridine-5-carboxamide;
3-(6-amino-5-cyanopyridin-3-yl)-N-(5-cyanopyridin-2-yl)-N-methylpyrazolo[1,5-a]pyridine-5-carboxamide;
3-(6-amino-5-chloropyridin-3-yl)-N-(5-cyanopyridin-2-yl)-N-methylpyrazolo[1,5-a]pyridine-5-carboxamide;
3-(6-amino-5-(dimethylcarbamoyl)pyridin-3-yl)-N-(5-cyanopyridin-2-yl)-N-methylpyrazolo[1,5-a]pyridine-5-carboxamide;
3-(6-amino-5-methoxypyridin-3-yl)-N-(5-cyanopyridin-2-yl)-N-methylpyrazolo[1,5-a]pyridine-5-carboxamide; and
3-(4-carbamoylphenyl)-N-(4-chloro-2-formylphenyl)-N-methylpyrazolo[1,5-a]pyridine-5-carboxamide.
N-(5-Cyanopyridin-2-yl)-N-ethyl-3-(4-(methylcarbamoyl)phenyl)pyrazolo[1,5-a]pyridine-5-carboxamide;
N-(5-cyanopyridin-2-yl)-N-isopropyl-3-(4-(methylcarbamoyl)phenyl)pyrazolo[1,5-a]pyridine-5-carboxamide;
5-(5-(1-(4-cyanophenyl)-2-methylhydrazinecarbonyl)pyrazolo[1,5-a]pyridin-3-yl)-N-methyl picolinamide;
N-(5-Cyanopyridin-2-yl)-N-cyclopropyl-3-(4-(methylcarbamoyl)phenyl)pyrazolo[1,5-a]pyridine-5-carboxamide;
5-Cyano-N-methyl-N-(3-(4-(methylcarbamoyl)phenyl)pyrazolo[1,5-a]pyridin-5-yl)picolinamide;
N-ethyl-N-(5-fluoropyridin-2-yl)-3-(4-(methylcarbamoyl)phenyl)pyrazolo[1,5-a]pyridine-5-carboxamide;
N-ethyl-3-(4-(methylcarbamoyl)phenyl)-N-(5-(trifluoromethyl)pyridin-2-yl)pyrazolo[1,5-a]pyridine-5-carboxamide;
N-(4-Cyanophenyl)-N-methyl-3-(6-(methylcarbamoyl)pyridin-3-yl)pyrazolo[1,5-a]pyridine-5-carboxamide;
3-(5-Amino-6-chloropyridin-3-yl)-N-(5-cyanopyridin-2-yl)-N-methylpyrazolo[1,5-a]pyridine-5-carboxamide;
N-(4-Chlorophenyl)-N-methyl-3-(4-(methylcarbamoyl)phenyl)pyrazolo[1,5-a]pyridine-5-carboxamide;
3-(4-Carbamoylphenyl)-N-(4-chlorophenyl)-N-methylpyrazolo[1,5-a]pyridine-5-carboxamide;
4-(5-((5-Cyanopyridin-2-yl)(methyl)carbamoyl)pyrazolo [1,5-a]pyridin-3-yl)benzoic acid;
N-(5-Cyanopyridin-2-yl)-3-(4-((2-hydroxyethyl)carbamoyl)phenyl)-N-methylpyrazolo[1,5-a]pyridine-5-carboxamide;
N-Methyl-3-(4-(methylcarbamoyl)phenyl)-N-(4-(trifluoromethyl)phenyl)pyrazolo[1,5-a]pyridine-5-carboxamide;
3-(4-((2-Aminoethyl)carbamoyl)phenyl)-N-(5-cyanopyridin-2-yl)-N-methylpyrazolo[1,5-a]pyridine-5-carboxamide;
3-(4-Carbamoylphenyl)-N-(4-cyanophenyl)-N-(2-hydroxyethyl)pyrazolo[1,5-a]pyridine-5-carboxamide;
N-(5-Cyanopyridin-2-yl)-3-(4-(methylcarbamoyl)phenyl)pyrazolo[1,5-a]pyridine-5-carboxamide;
3-(6-Chloro-5-(methylsulfonamido) pyridin-3-yl)-N-(5-cyanopyridin-2-yl)-N-methylpyrazolo[1,5-a]pyridine-5-carboxamide;
3-(2-Aminopyridin-4-yl)-N-(5-cyanopyridin-2-yl)-N-methylpyrazolo[1,5-a]pyridine-5-carboxamide;
N-(4-Chlorophenyl)-N-methyl-3-(6-(methylcarbamoyl)pyridin-3-yl)pyrazolo[1,5-a]pyridine-5-carboxamide;
N-(4-Cyanophenyl)-N-(2-hydroxyethyl)-3-(4-(methylcarbamoyl)phenyl)pyrazolo[1,5-a]pyridine-5-carboxamide;
N-(5-(2-Aminoethoxy)pyridin-2-yl)-N-methyl-3-(4-(methylcarbamoyl)phenyl)pyrazolo[1,5-a]pyridine-5-carboxamide;
N-(5-Cyanopyridin-2-yl)-N-cyclobutyl-3-(4-(methylcarbamoyl)phenyl)pyrazolo[1,5-a]pyridine-5-carboxamide;
N-(5-Cyanopyridin-2-yl)-N-methyl-3-(4-(piperidin-4-ylcarbamoyl)phenyl)pyrazolo[1,5-a]pyridine-5-carboxamide;
N-(5-Cyanopyridin-2-yl)-N-methyl-3-(4-((2-(methylamino)ethyl)carbamoyl)phenyl)pyrazolo [1,5-a]pyridine-5-carboxamide;
N-(5-cyanopyridin-2-yl)-3-(4-((2-(dimethylamino)ethyl)carbamoyl)phenyl)-N-methylpyrazolo[1,5-a]pyridine-5-carboxamide;
N-(4-Chlorophenyl)-N-cyclopropyl-3-(4-(methylcarbamoyl)phenyl)pyrazolo[1,5-a]pyridine-5-carboxamide;
N-(5-Cyanopyridin-2-yl)-N-(cyclopropylmethyl)-3-(4-(methylcarbamoyl)phenyl)pyrazolo[1,5-a]pyridine-5-carboxamide;
N-(4-Cyanophenyl)-N-cyclopropyl-3-(4-(methylcarbamoyl)phenyl)pyrazolo[1,5-a]pyridine-5-carboxamide;
N-(tert-Butyl)-N-(5-cyanopyridin-2-yl)-3-(4-(methylcarbamoyl)phenyl)pyrazolo[1,5-a]pyridine-5-carboxamide;
3-(4-Carbamoylphenyl)-N-(5-cyanopyridin-2-yl)-N-cyclopropylpyrazolo[1,5-a]pyridine-5-carboxamide;
N-(5-Cyanopyridin-2-yl)-N-cyclopropyl-3-(4-(isopropylcarbamoyl)phenyl)pyrazolo[1,5-a]pyridine-5-carboxamide;
N-(6-Methoxypyridin-3-yl)-N-methyl-3-(4-(methylcarbamoyl)phenyl)pyrazolo[1,5-a]pyridine-5-carboxamide;
N-(5-Cyanopyridin-2-yl)-N-cyclopropyl-3-(4-(cyclopropylcarbamoyl)phenyl)pyrazolo[1,5a]pyridine-5-carboxamide;
N-(5-Chloropyridin-2-yl)-N-cyclopropyl-3-(4-(methylcarbamoyl)phenyl)pyrazolo[1,5-a]pyridine-5-carboxamide;
N-Cyclopropyl-N-(5-fluoropyridin-2-yl)-3-(4-(methylcarbamoyl)phenyl)pyrazolo[1,5a]pyridine-5-carboxamide;
N-(5-Cyanopyridin-2-yl)-N-cyclopentyl-3-(4-(methylcarbamoyl)phenyl)pyrazolo[1,5-a]pyridine-5-carboxamide;
N-(5-Cyanopyridin-2-yl)-N-cyclopropyl-3-(4-(ethylcarbamoyl)phenyl)pyrazolo[1,5-a]pyridine-5-carboxamide;
6-(N-Cyclopropyl-3-(4-(methylcarbamoyl)phenyl)pyrazolo[1,5-a]pyridine-5-carboxamido) nicotinic acid;
N-(5-Carbamoylpyridin-2-yl)-N-cyclopropyl-3-(4-(methylcarbamoyl)phenyl)pyrazolo[1,5-a]pyridine-5-carboxamide;
N-Cyclopropyl-N-(3,4-difluorophenyl)-3-(4-(methylcarbamoyl)phenyl)pyrazolo[1,5-a]pyridine-5-carboxamide;
N-(5-Cyanopyridin-2-yl)-N-cyclopropyl-3-(4-(oxetan-3-ylcarbamoyl)phenyl)pyrazolo[1,5-a]pyridine-5-carboxamide;
N-Cyclopropyl-3-(4-(methylcarbamoyl)phenyl)-N-(5-(trifluoromethyl)pyridin-2-yl)pyrazolo [1,5-a]pyridine-5-carboxamide;
N-(5-Cyanopyridin-2-yl)-N-cyclopropyl-3-(5-(methylcarbamoyl)pyridin-2-yl)pyrazolo[1,5-a]pyridine-5-carboxamide;
3-(4-carbamoylphenyl)-N-(4-cyanophenyl)-N-cyclopropylpyrazolo[1,5-a]pyridine-5-carboxamide;
3-(4-Carbamoylphenyl)-N-cyclopropyl-N-(3,4-difluorophenyl)pyrazolo[1,5-a]pyridine-5-carboxamide;
N-Cyclopropyl-3-(4-(methylcarbamoyl)phenyl)-N-(5-methylpyridin-2-yl)pyrazolo[1,5-a]pyridine-5-carboxamide;
N-(4-Cyanophenyl)-N-cyclopropyl-3-(6-(methylcarbamoyl)pyridin-3-yl)pyrazolo[1,5-a]pyridine-5-carboxamide;
3-(6-Carbamoylpyridin-3-yl)-N-(4-cyanophenyl)-N-cyclopropylpyrazolo[1,5-a]pyridine-5-carboxamide;
3-(5-Carbamoylpyridin-2-yl)-N-(5-cyanopyridin-2-yl)-N-cyclopropylpyrazolo[1,5-a]pyridine-5-carboxamide;
N-(5-cyanopyridin-2-yl)-N-ethyl-3-(4-[N-methylsulfamoyl]phenyl)pyrazolo[1,5-a]pyridine-5-carboxamide;
N-(4-Cyanophenyl)-N-cyclopropyl-3-(5-(methylcarbamoyl)pyridin-2-yl)pyrazolo[1,5-a]pyridine-5-carboxamide;
3-(5-Carbamoylpyridin-2-yl)-N-(4-cyanophenyl)-N-cyclopropylpyrazolo[1,5-a]pyridine-5-carboxamide;
N-(4-Chlorophenyl)-N-cyclopropyl-3-(6-(methylcarbamoyl)pyridin-3-yl)pyrazolo[1,5-a]pyridine-5-carboxamide;
N-(5-Cyanopyridin-2-yl)-N-cyclobutyl-3-(6-(methylcarbamoyl)pyridin-3-yl)pyrazolo[1,5-a]pyridine-5-carboxamide;
N-(4-Cyanophenyl)-N-cyclobutyl-3-(6-(methylcarbamoyl)pyridin-3-yl)pyrazolo[1,5-a]pyridine-5-carboxamide;
N-(4-Cyanophenyl)-N-isopropyl-3-(6-(methylcarbamoyl)pyridin-3-yl)pyrazolo[1,5-a]pyridine-5-carboxamide;
5-Cyano-N-cyclopropyl-N-(3-(4-(methylcarbamoyl)phenyl)pyrazolo[1,5-a]pyridin-5-yl) picolinamide;
N-(5-Cyanopyridin-2-yl)-N-isopropyl-3-(6-(methylcarbamoyl)pyridin-3-yl)pyrazolo[1,5-a]pyridine-5-carboxamide;
N-(5-Cyano-6-methoxypyridin-2-yl)-N-cyclopropyl-3-(4-(methylcarbamoyl)phenyl)pyrazolo [1,5-a]pyridine-5-carboxamide;
N-(5-cyanopyridin-2-yl)-N-ethyl-3-(6-[methylcarbamoyl]pyridin-3-yl)pyrazolo[1,5-a]pyridine-5-carboxamide;
N-(5-fluoropyridin-2-yl)-N-isopropyl-3-(4-(methylcarbamoyl)phenyl)pyrazolo[1,5-a]pyridine-5-carboxamide;
N-Isopropyl-3-(6-(methylcarbamoyl)pyridin-3-yl)-N-(5-(trifluoromethyl)pyridin-2-yl)pyrazolo [1,5-a]pyridine-5-carboxamide;
N-Isopropyl-3-(4-(methylcarbamoyl)phenyl)-N-(5-(trifluoromethyl)pyridin-2-yl)pyrazolo[1,5-a]pyridine-5-carboxamide;
N-(5-cyanopyridin-2-yl)-N-isopropyl-3-(5-(methylcarbamoyl)pyridin-2-yl)pyrazolo[1,5-a]pyridine-5-carboxamide;
N-(5-Cyanopyridin-2-yl)-N-cyclobutyl-3-(5-(methylcarbamoyl)pyridin-2-yl)pyrazolo[1,5-a]pyridine-5-carboxamide;
N-(5-fluoropyridin-2-yl)-N-isopropyl-3-(6-(methylcarbamoyl)pyridin-3-yl)pyrazolo[1,5-a]pyridine-5-carboxamide;
N-ethyl-3-(6-(methylcarbamoyl)pyridin-3-yl)-N-(5-(trifluoromethyl)pyridin-2-yl)pyrazolo[1,5-a]pyridine-5-carboxamide;
N-(5-cyanopyridin-2-yl)-N-ethyl-3-(4-(methylsulfonyl)phenyl)pyrazolo[1,5-a]pyridine-5-carboxamide;
N-ethyl-N-(5-fluoropyridin-2-yl)-3-(6-(methylcarbamoyl)pyridin-3-yl)pyrazolo[1,5-a]pyridine-5-carboxamide;
N-cyclobutyl-3-(4-(methylcarbamoyl)phenyl)-N-(5-(trifluoromethyl)pyridin-2-yl)pyrazolo[1,5-a]pyridine-5-carboxamide;
N N-cyclobutyl-3-(6-(methylcarbamoyl)pyridin-3-yl)-N-(5-(trifluoromethyl)pyridin-2-yl)pyrazolo[1,5-a]pyridine-5-carboxamide;
N-(5-cyano-6-(2-hydroxyethoxy)pyridin-2-yl)-N-ethyl-3-(4-(methylcarbamoyl)phenyl)pyrazolo[1,5-a]pyridine-5-carboxamide;
4-(5-(N-(5-Cyanopyridin-2-yl)-N-methylsulfamoyl)pyrazolo[1,5-a]pyridin-3-yl)-N-methyl benzamide;
4-(5-(N-(5-Cyanopyridin-2-yl)-N-cyclopropylsulfamoyl)pyrazolo[1,5-a]pyridin-3-yl)-N-methylbenzamide; and
4-(5-(N-(5-cyanopyridin-2-yl)-N-cyclopropylsulfamoyl)pyrazolo[1,5-a]pyridin-3-yl)benzamide.

13. A method for treating, inhibiting, ameliorating, or eradicating the pathology and/or symptomology of cryptosporidiosis caused by a cryptosporidium protozoa, comprising administering to a subject in need thereof a therapeutically effective amount of an agent capable of modulating or inhibiting the activity of a phosphatidylinositol-4-OH kinase (PI4K) of said protozoa.

14. The method of claim 13, wherein the crypotosporidium protozoa is Cryptosporidium hominis or Cryptosporidium parvum.

15. The method of claim 13, wherein the agent is a compound of claim according to Formula I, 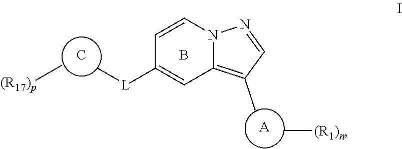
or a pharmaceutical acceptable salt, tautomer or stereoisomer thereof, wherein
n is 0, 1,2 or 3;
p is 0, 1, 2, or 3;
L is selected from the group consisting of *—(CHR₃)₁₋₃—, *—CHR₃N(R₂)—, *—CHR₃O—, *—CHR₃S—, *—CHR₃S(O)—, *—CHR₃N(R₂)CHR₃—, *—C(O)—, *—C(O)N(R₂)—, *—C(O)N(R₂)CHR₃—, *—N(R₂)—, *—N(R₂)CHR₃—, *—N(R₂)C(O)—, *—N(R₂)C(O)N(R₂)—, *—N(R₂)S(O)₂—, and *—S(O)₂N(R₂)—, wherein
* represents the point of attachment of L to the pyrazolo[1,5-a]pyridine fused ring depicted in Formula I (Ring B);
each R₂ is selected from the group consisting of hydrogen, C₁₋₆alkyl, haloC₁₋₆alkyl, R—C₀₋₄alkylene, and R—C₀₋₄alkylene-C(O)—, wherein R is selected from the group consisting of hydroxyl, C₁₋₄alkoxy, amino, C₁₋₄alkylamino, C₃₋₆cycloalkyl, C₄₋₆heterocycloalkyl, and C₅₋₆heteroaryl, wherein the C₃₋₆cycloalkyl, C₄₋₆heterocycloalkyl, or C₅₋₆heteroaryl of R is unsubstituted or substituted by 1-2 substituents independently selected from the group consisting of halo, amino, hydroxyl, C₁₋₄alkyl, C₁₋₄alkoxy, oxo, and C₅₋₆heteroaryl; and
R₃ is hydrogen or C₁₋₄alkyl;
Ring A is C₆₋₁₀aryl or C₅₋₁₀heteroaryl;
Ring C is selected from the group consisting of C₆₋₁₀aryl, C₅₋₁₀heteroaryl, C₅₋₇cycloalkyl, C₅₋₇heterocycloalkyl, and a fused bicyclyl comprising a C₅₋₆heterocycloalky fused to a phenyl;
each R₁ is independently selected from the group consisting of halo, cyano, amino, C₁₋₄alkyl, C₁₋₄alkoxy, halo-C₁₋₄alkyl, —C(O)NR₇R₈, —NHC(O)R₁₁, phenyl, C₅₋₆heteroaryl, —C(O)R₁₁, —NHS(O)₂R₁₁, —S(O)₂R₁₁, and —S(O)₂NHR₈, wherein
the phenyl or C₅₋₆heteroaryl of R₁ is unsubstituted or substituted by 1-2 substituents independently selected from the group consisting of C₁₋₄alkyl, amino, halo, and C₁₋₄alkylamino;
R₇ is selected from the group consisting of hydrogen, C₁₋₄alkyl and haloC₁₋₄alkyl;
R₈ is selected from the group consisting of hydrogen; haloC₁₋₄alkyl; C₃₋₆cycloalkyl; C₄₋₆heterocycloalkyl C₁₋₄alkyl unsubstituted or substituted by hydroxy, amino, or C₁₋₄alkylamino; and
R₁₁ is selected from the group consisting of hydroxyl and C₁₋₆alkyl unsubstituted or substituted by 1-2 substituents independently selected from the group consisting of amino, C₃₋₆cycloalkyl, and C₄₋₆heterocycloalkyl;
each R₁₇ is selected from the group consisting of cyano, halo, C₁₋₄alkyl, halo-C₁₋₄alkyl, oxo, C₃₋₆cycloalkyl, —S(O)₂C₁₋₄alkyl; C₁₋₄alkoxy unsubstituted or substituted by hydroxy or amino; and —C(O)R₁₂, wherein R₁₂ is hydrogen, hydroxy or amino.
